SEQUENCE LISTING (68 sequences)

SEQ ID NO: 1 - protein, 424 aa, Homo sapiens
MGSLVLTLCALFCLAAYLVSGSPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRRNAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 2 - DNA, 1272 bp, Homo sapiens
atgggctctctggtcctgacactgtgcgctcttttctgcctggcagcttacttggtttctggcagccccatcatgaacctagagcagtctcctctggaagaagatatgtccctctttggtgatgttttctcagagcaagacggtgtcgactttaacacactgctccagagcatgaaggatgagtttcttaagacactaaacctctctgacatccccacgcaggattcagccaaggtggacccaccagagtacatgttggaactctacaacaaatttgcaacagatcggacctccatgccctctgccaacatcattaggagtttcaagaatgaagatctgttttcccagccggtcagttttaatgggctccgaaaataccccctcctcttcaatgtgtccattcctcaccatgaagaggtcatcatggctgaacttaggctatacacactggtgcaaagggatcgtatgatatacgatggagtagaccggaaaattaccatttttgaagtgctggagagcaaaggggataatgagggagaaagaaacatgctggtcttggtgtctggggagatatatggaaccaacagtgagtgggagacttttgatgtcacagatgccatcagacgttggcaaaagtcaggctcatccacccaccagctggaggtccacattgagagcaaacacgatgaagctgaggatgccagcagtggacggctagaaatagataccagtgcccagaataagcataaccctttgctcatcgtgttttctgatgaccaaagcagtgacaaggagaggaaggaggaactgaatgaaatgatttcccatgagcaacttccagagctggacaacttgggcctggatagcttttccagtggacctggggaagaggctttgttgcagatgagatcaaacatcatctatgactccactgcccgaatcagaaggaacgccaaaggaaactactgtaagaggaccccgctctacatcgacttcaaggagattgggtgggactcctggatcatcgctccgcctggatacgaagcctatgaatgccgtggtgtttgtaactaccccctggcagagcatctcacacccacaaagcatgcaattatccaggccttggtccacctcaagaattcccagaaagcttccaaagcctgctgtgtgcccacaaagctagagcccatctccatcctctatttagacaaaggcgtcgtcacctacaagtttaaatacgaaggcatggccgtctccgaatgtggctgtaga

SEQ ID NO: 3 - protein, 296 aa, Homo sapiens
GSPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRR

SEQ ID NO: 4 - DNA, 885 bp, Homo sapiens
agccccatcatgaacctagagcagtctcctctggaagaagatatgtccctctttggtgatgttttctcagagcaagacggtgtcgactttaacacactgctccagagcatgaaggatgagtttcttaagacactaaacctctctgacatccccacgcaggattcagccaaggtggacccaccagagtacatgttggaactctacaacaaatttgcaacagatcggacctccatgccctctgccaacatcattaggagtttcaagaatgaagatctgttttcccagccggtcagttttaatgggctccgaaaataccccctcctcttcaatgtgtccattcctcaccatgaagaggtcatcatggctgaacttaggctatacacactggtgcaaagggatcgtatgatatacgatggagtagaccggaaaattaccatttttgaagtgctggagagcaaaggggataatgagggagaaagaaacatgctggtcttggtgtctggggagatatatggaaccaacagtgagtgggagacttttgatgtcacagatgccatcagacgttggcaaaagtcaggctcatccacccaccagctggaggtccacattgagagcaaacacgatgaagctgaggatgccagcagtggacggctagaaatagataccagtgcccagaataagcataaccctttgctcatcgtgttttctgatgaccaaagcagtgacaaggagaggaaggaggaactgaatgaaatgatttcccatgagcaacttccagagctggacaacttgggcctggatagcttttccagtggacctggggaagaggctttgttgcagatgagatcaaacatcatctatgactccactgcccgaatcagaagg

SEQ ID NO: 5 - protein, 1038 aa, Homo sapiens
MTSSLQRPWRVPWLPWTILLVSTAAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAASEPSLDLDNLKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELMMIWERNKSVSPTVNPMSTAMQNERNLSHNRRVPKIGPYPDYSSSSYIEDSIHHTDSIVKNISSEHSMSSTPLTIGEKNRNSINYERQQAQARIPSPETSVTSLSTNTTTTNTTGLTPSTGMTTISEMPYPDETNLHTTNVAQSIGPTPVCLQLTEEDLETNKLDPKEVDKNLKESSDENLMEHSLKQFSGPDPLSSTSSSLLYPLIKLAVEATGQQDFTQTANGQACLIPDVLPTQIYPLPKQQNLPKRPTSLPLNTKNSTKEPRLKFGSKHKSNLKQVETGVAKMNTINAAEPHVVTVTMNGVAGRNHSVNSHAATTQYANGTVLSGQTTNIVTHRAQEMLQNQFIGEDTRLNINSSPDEHEPLLRREQQAGHDEGVLDRLVDRRERPLEGGRTNSNNNNSNPCSEQDVLAQGVPSTAADPGPSKPRRAQRPNSLDLSATNVLDGSSIQIGESTQDGKSGSGEKIKKRVKTPYSLKRWRPSTWVISTESLDCEVNNNGSNRAVHSKSSTAVYLAEGGTATTMVSKDIGMNCL

SEQ ID NO: 6 - protein, 124 aa, Artificial Sequence [sequence omitted]

SEQ ID NO: 7 - DNA, 3114 bp, Homo sapiens
atgacttcctcgctgcagcggccctggcgggtgccctggctaccatggaccatcctgctggtcagcactgcggctgcttcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagacaataatcattgctttggcatcagtctctgtattagctgttttgatagttgccttatgctttggatacagaatgttgacaggagaccgtaaacaaggtcttcacagtatgaacatgatggaggcagcagcatccgaaccctctcttgatctagataatctgaaactgttggagctgattggccgaggtcgatatggagcagtatataaaggctccttggatgagcgtccagttgctgtaaaagtgttttcctttgcaaaccgtcagaattttatcaacgaaaagaacatttacagagtgcctttgatggaacatgacaacattgcccgctttatagttggagatgagagagtcactgcagatggacgcatggaatatttgcttgtgatggagtactatcccaatggatctttatgcaagtatttaagtctccacacaagtgactgggtaagctcttgccgtcttgctcattctgttactagaggactggcttatcttcacacagaattaccacgaggagatcattataaacctgcaatttcccatcgagatttaaacagcagaaatgtcctagtgaaaaatgatggaacctgtgttattagtgactttggactgtccatgaggctgactggaaatagactggtgcgcccaggggaggaagataatgcagccataagcgaggttggcactatcagatatatggcaccagaagtgctagaaggagctgtgaacttgagggactgtgaatcagctttgaaacaagtagacatgtatgctcttggactaatctattgggagatatttatgagatgtacagacctcttcccaggggaatccgtaccagagtaccagatggcttttcagacagaggttggaaaccatcccacttttgaggatatgcaggttctcgtgtctagggaaaaacagagacccaagttcccagaagcctggaaagaaaatagcctggcagtgaggtcactcaaggagacaatcgaagactgttgggaccaggatgcagaggctcggcttactgcacagtgtgctgaggaaaggatggctgaacttatgatgatttgggaaagaaacaaatctgtgagcccaacagtcaatccaatgtctactgctatgcagaatgaacgcaacctgtcacataataggcgtgtgccaaaaattggtccttatccagattattcttcctcctcatacattgaagactctatccatcatactgacagcatcgtgaagaatatttcctctgagcattctatgtccagcacacctttgactataggggaaaaaaaccgaaattcaattaactatgaacgacagcaagcacaagctcgaatccccagccctgaaacaagtgtcaccagcctctccaccaacacaacaaccacaaacaccacaggactcacgccaagtactggcatgactactatatctgagatgccatacccagatgaaacaaatctgcataccacaaatgttgcacagtcaattgggccaacccctgtctgcttacagctgacagaagaagacttggaaaccaacaagctagacccaaaagaagttgataagaacctcaaggaaagctctgatgagaatctcatggagcactctcttaaacagttcagtggcccagacccactgagcagtactagttctagcttgctttacccactcataaaacttgcagtagaagcaactggacagcaggacttcacacagactgcaaatggccaagcatgtttgattcctgatgttctgcctactcagatctatcctctccccaagcagcagaaccttcccaagagacctactagtttgcctttgaacaccaaaaattcaacaaaagagccccggctaaaatttggcagcaagcacaaatcaaacttgaaacaagtcgaaactggagttgccaagatgaatacaatcaatgcagcagaacctcatgtggtgacagtcaccatgaatggtgtggcaggtagaaaccacagtgttaactcccatgctgccacaacccaatatgccaatgggacagtactatctggccaaacaaccaacatagtgacacatagggcccaagaaatgttgcagaatcagtttattggtgaggacacccggctgaatattaattccagtcctgatgagcatgagcctttactgagacgagagcaacaagctggccatgatgaaggtgttctggatcgtcttgtggacaggagggaacggccactagaaggtggccgaactaattccaataacaacaacagcaatccatgttcagaacaagatgttcttgcacagggtgttccaagcacagcagcagatcctgggccatcaaagcccagaagagcacagaggcctaattctctggatctttcagccacaaatgtcctggatggcagcagtatacagataggtgagtcaacacaagatggcaaatcaggatcaggtgaaaagatcaagaaacgtgtgaaaactccctattctcttaagcggtggcgcccctccacctgggtcatctccactgaatcgctggactgtgaagtcaacaataatggcagtaacagggcagttcattccaaatccagcactgctgtttaccttgcagaaggaggcactgctacaaccatggtgtctaaagatataggaatgaactgtctg

SEQ ID NO: 8 - DNA, 372 bp, Artificial Sequence
  Description of Artificial Sequence: Synthetic polynucleotide
tcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagaca

SEQ ID NO: 9 - protein, 530 aa, Homo sapiens
MTSSLQRPWRVPWLPWTILLVSTAAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAASEPSLDLDNLKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELMMIWERNKSVSPTVNPMSTAMQNERR

SEQ ID NO: 10 - DNA, 1590 bp, Homo sapiens
atgacttcctcgctgcagcggccctggcgggtgccctggctaccatggaccatcctgctggtcagcactgcggctgcttcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagacaataatcattgctttggcatcagtctctgtattagctgttttgatagttgccttatgctttggatacagaatgttgacaggagaccgtaaacaaggtcttcacagtatgaacatgatggaggcagcagcatccgaaccctctcttgatctagataatctgaaactgttggagctgattggccgaggtcgatatggagcagtatataaaggctccttggatgagcgtccagttgctgtaaaagtgttttcctttgcaaaccgtcagaattttatcaacgaaaagaacatttacagagtgcctttgatggaacatgacaacattgcccgctttatagttggagatgagagagtcactgcagatggacgcatggaatatttgcttgtgatggagtactatcccaatggatctttatgcaagtatttaagtctccacacaagtgactgggtaagctcttgccgtcttgctcattctgttactagaggactggcttatcttcacacagaattaccacgaggagatcattataaacctgcaatttcccatcgagatttaaacagcagaaatgtcctagtgaaaaatgatggaacctgtgttattagtgactttggactgtccatgaggctgactggaaatagactggtgcgcccaggggaggaagataatgcagccataagcgaggttggcactatcagatatatggcaccagaagtgctagaaggagctgtgaacttgagggactgtgaatcagctttgaaacaagtagacatgtatgctcttggactaatctattgggagatatttatgagatgtacagacctcttcccaggggaatccgtaccagagtaccagatggcttttcagacagaggttggaaaccatcccacttttgaggatatgcaggttctcgtgtctagggaaaaacagagacccaagttcccagaagcctggaaagaaaatagcctggcagtgaggtcactcaaggagacaatcgaagactgttgggaccaggatgcagaggctcggcttactgcacagtgtgctgaggaaaggatggctgaacttatgatgatttgggaaagaaacaaatctgtgagcccaacagtcaatccaatgtctactgctatgcagaatgaacgtagg

SEQ ID NO: 11 - protein, 503 aa, Homo sapiens
MTLGSPRKGLLMLLMALVTQGDPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLALILGPVLALLALVALGVLGLWHVRRRQEKQRGLHSELGESSLILKASEQGDSMLGDLLDSDCTTGSGSGLPFLVQRTVARQVALVECVGKGRYGEVWRGLWHGESVAVKIFSSRDEQSWFRETEIYNTVLLRHDNILGFIASDMTSRNSSTQLWLITHYHEHGSLYDFLQRQTLEPHLALRLAVSAACGLAHLHVEIFGTQGKPAIAHRDFKSRNVLVKSNLQCCIADLGLAVMHSQGSDYLDIGNNPRVGTKRYMAPEVLDEQIRTDCFESYKWTDIWAFGLVLWEIARRTIVNGIVEDYRPPFYDVVPNDPSFEDMKKVVCVDQQTPTIPNRLAADPVLSGLAQMMRECWYPNPSARLTALRIKKTLQKISNSPEKPKVIQ

SEQ ID NO: 12 - protein, 97 aa, Artificial Sequence
  Description of Artificial Sequence: Synthetic polypeptide
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQ

SEQ ID NO: 13 - DNA, 1509 bp, Homo sapiens
atgaccttgggctcccccaggaaaggccttctgatgctgctgatggccttggtgacccagggagaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcagggggcgccccaccgagttcgtcaaccactactgctgcgacagccacctctgcaaccacaacgtgtccctggtgctggaggccacccaacctccttcggagcagccgggaacagatggccagctggccctgatcctgggccccgtgctggccttgctggccctggtggccctgggtgtcctgggcctgtggcatgtccgacggaggcaggagaagcagcgtggcctgcacagcgagctgggagagtccagtctcatcctgaaagcatctgagcagggcgacagcatgttgggggacctcctggacagtgactgcaccacagggagtggctcagggctccccttcctggtgcagaggacagtggcacggcaggttgccttggtggagtgtgtgggaaaaggccgctatggcgaagtgtggcggggcttgtggcacggtgagagtgtggccgtcaagatcttctcctcgagggatgaacagtcctggttccgggagactgagatctataacacagtgttgctcagacacgacaacatcctaggcttcatcgcctcagacatgacctcccgcaactcgagcacgcagctgtggctcatcacgcactaccacgagcacggctccctctacgactttctgcagagacagacgctggagccccatctggctctgaggctagctgtgtccgcggcatgcggcctggcgcacctgcacgtggagatcttcggtacacagggcaaaccagccattgcccaccgcgacttcaagagccgcaatgtgctggtcaagagcaacctgcagtgttgcatcgccgacctgggcctggctgtgatgcactcacagggcagcgattacctggacatcggcaacaacccgagagtgggcaccaagcggtacatggcacccgaggtgctggacgagcagatccgcacggactgctttgagtcctacaagtggactgacatctgggcctttggcctggtgctgtgggagattgcccgccggaccatcgtgaatggcatcgtggaggactatagaccacccttctatgatgtggtgcccaatgaccccagctttgaggacatgaagaaggtggtgtgtgtggatcagcagacccccaccatccctaaccggctggctgcagacccggtcctctcaggcctagctcagatgatgcgggagtgctggtacccaaacccctctgcccgactcaccgcgctgcggatcaagaagacactacaaaaaattagcaacagtccagagaagcctaaagtgattcaa

SEQ ID NO: 14 - DNA, 291 bp, Artificial Sequence
  Description of Artificial Sequence: Synthetic polynucleotide
gaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcagggggcgccccaccgagttcgtcaaccactactgctgcgacagccacctctgcaaccacaacgtgtccctggtgctggaggccacccaacctccttcggagcagccgggaacagatggccag

SEQ ID NO: 15 - protein, 658 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

SEQ ID NO: 16 - DNA, 1974 bp, Homo sapiens
atggaccgcggcacgctccctctggctgttgccctgctgctggccagctgcagcctcagccccacaagtcttgcagaaacagtccattgtgaccttcagcctgtgggccccgagaggggcgaggtgacatataccactagccaggtctcgaagggctgcgtggctcaggcccccaatgccatccttgaagtccatgtcctcttcctggagttcccaacgggcccgtcacagctggagctgactctccaggcatccaagcaaaatggcacctggccccgagaggtgcttctggtcctcagtgtaaacagcagtgtcttcctgcatctccaggccctgggaatcccactgcacttggcctacaattccagcctggtcaccttccaagagcccccgggggtcaacaccacagagctgccatccttccccaagacccagatccttgagtgggcagctgagaggggccccatcacctctgctgctgagctgaatgacccccagagcatcctcctccgactgggccaagcccaggggtcactgtccttctgcatgctggaagccagccaggacatgggccgcacgctcgagtggcggccgcgtactccagccttggtccggggctgccacttggaaggcgtggccggccacaaggaggcgcacatcctgagggtcctgccgggccactcggccgggccccggacggtgacggtgaaggtggaactgagctgcgcacccggggatctcgatgccgtcctcatcctgcagggtcccccctacgtgtcctggctcatcgacgccaaccacaacatgcagatctggaccactggagaatactccttcaagatctttccagagaaaaacattcgtggcttcaagctcccagacacacctcaaggcctcctgggggaggcccggatgctcaatgccagcattgtggcatccttcgtggagctaccgctggccagcattgtctcacttcatgcctccagctgcggtggtaggctgcagacctcacccgcaccgatccagaccactcctcccaaggacacttgtagcccggagctgctcatgtccttgatccagacaaagtgtgccgacgacgccatgaccctggtactaaagaaagagcttgttgcgcatttgaagtgcaccatcacgggcctgaccttctgggaccccagctgtgaggcagaggacaggggtgacaagtttgtcttgcgcagtgcttactccagctgtggcatgcaggtgtcagcaagtatgatcagcaatgaggcggtggtcaatatcctgtcgagctcatcaccacagcggaaaaaggtgcactgcctcaacatggacagcctctctttccagctgggcctctacctcagcccacacttcctccaggcctccaacaccatcgagccggggcagcagagctttgtgcaggtcagagtgtccccatccgtctccgagttcctgctccagttagacagctgccacctggacttggggcctgagggaggcaccgtggaactcatccagggccgggcggccaagggcaactgtgtgagcctgctgtccccaagccccgagggtgacccgcgcttcagcttcctcctccacttctacacagtacccatacccaaaaccggcaccctcagctgcacggtagccctgcgtcccaagaccgggtctcaagaccaggaagtccataggactgtcttcatgcgcttgaacatcatcagccctgacctgtctggttgcacaagcaaaggcctcgtcctgcccgccgtgctgggcatcacctttggtgccttcctcatcggggccctgctcactgctgcactctggtacatctactcgcacacgcgttcccccagcaagcgggagcccgtggtggcggtggctgccccggcctcctcggagagcagcagcaccaaccacagcatcgggagcacccagagcaccccctgctccaccagcagcatggca

SEQ ID NO: 17 - protein, 625 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTREYPRPPQ

SEQ ID NO: 18 - DNA, 1875 bp, Homo sapiens
atggaccgcggcacgctccctctggctgttgccctgctgctggccagctgcagcctcagccccacaagtcttgcagaaacagtccattgtgaccttcagcctgtgggccccgagaggggcgaggtgacatataccactagccaggtctcgaagggctgcgtggctcaggcccccaatgccatccttgaagtccatgtcctcttcctggagttcccaacgggcccgtcacagctggagctgactctccaggcatccaagcaaaatggcacctggccccgagaggtgcttctggtcctcagtgtaaacagcagtgtcttcctgcatctccaggccctgggaatcccactgcacttggcctacaattccagcctggtcaccttccaagagcccccgggggtcaacaccacagagctgccatccttccccaagacccagatccttgagtgggcagctgagaggggccccatcacctctgctgctgagctgaatgacccccagagcatcctcctccgactgggccaagcccaggggtcactgtccttctgcatgctggaagccagccaggacatgggccgcacgctcgagtggcggccgcgtactccagccttggtccggggctgccacttggaaggcgtggccggccacaaggaggcgcacatcctgagggtcctgccgggccactcggccgggccccggacggtgacggtgaaggtggaactgagctgcgcacccggggatctcgatgccgtcctcatcctgcagggtcccccctacgtgtcctggctcatcgacgccaaccacaacatgcagatctggaccactggagaatactccttcaagatctttccagagaaaaacattcgtggcttcaagctcccagacacacctcaaggcctcctgggggaggcccggatgctcaatgccagcattgtggcatccttcgtggagctaccgctggccagcattgtctcacttcatgcctccagctgcggtggtaggctgcagacctcacccgcaccgatccagaccactcctcccaaggacacttgtagcccggagctgctcatgtccttgatccagacaaagtgtgccgacgacgccatgaccctggtactaaagaaagagcttgttgcgcatttgaagtgcaccatcacgggcctgaccttctgggaccccagctgtgaggcagaggacaggggtgacaagtttgtcttgcgcagtgcttactccagctgtggcatgcaggtgtcagcaagtatgatcagcaatgaggcggtggtcaatatcctgtcgagctcatcaccacagcggaaaaaggtgcactgcctcaacatggacagcctctctttccagctgggcctctacctcagcccacacttcctccaggcctccaacaccatcgagccggggcagcagagctttgtgcaggtcagagtgtccccatccgtctccgagttcctgctccagttagacagctgccacctggacttggggcctgagggaggcaccgtggaactcatccagggccgggcggccaagggcaactgtgtgagcctgctgtccccaagccccgagggtgacccgcgcttcagcttcctcctccacttctacacagtacccatacccaaaaccggcaccctcagctgcacggtagccctgcgtcccaagaccgggtctcaagaccaggaagtccataggactgtcttcatgcgcttgaacatcatcagccctgacctgtctggttgcacaagcaaaggcctcgtcctgcccgccgtgctgggcatcacctttggtgccttcctcatcggggccctgctcactgctgcactctggtacatctactcgcacacgcgtgagtaccccaggcccccacag

SEQ ID NO: 19 - protein, 582 aa, Artificial Sequence
  Description of Artificial Sequence: Synthetic polypeptide
ETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 569 aa, Artificial Sequence
  Description of Artificial Sequence: Synthetic polypeptide
ETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLITGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 563 aa, Artificial Sequence
  Description of Artificial Sequence: Synthetic polypeptide
ETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLITGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 550 aa, Artificial Sequence
  Description of Artificial Sequence: Synthetic polypeptide
ETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 25 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 26 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 27 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 3 aa, Artificial Sequence
  Description of Artificial Sequence: Synthetic peptide
GGG

SEQ ID NO: 29 - protein, 4 aa, Artificial Sequence
  Description of Artificial Sequence: Synthetic peptide
GGGG

SEQ ID NO: 30 - protein, 5 aa, Artificial Sequence
  Description of Artificial Sequence: Synthetic peptide
TGGGG

SEQ ID NO: 31 - protein, 5 aa, Artificial Sequence
  Description of Artificial Sequence: Synthetic peptide
SGGGG

SEQ ID NO: 32 - protein, 4 aa, Artificial Sequence
  Description of Artificial Sequence: Synthetic peptide
TGGG

SEQ ID NO: 33 - protein, 4 aa, Artificial Sequence
  Description of Artificial Sequence: Synthetic peptide
SGGG

SEQ ID NO: 34 - protein, 5 aa, Artificial Sequence
  Description of Artificial Sequence: Synthetic peptide
GGGGS

SEQ ID NO: 35 - protein, 10 aa, Artificial Sequence
  Description of Artificial Sequence: Synthetic peptide
GGGGSGGGGS

SEQ ID NO: 36 - protein, 11 aa, Artificial Sequence
  Description of Artificial Sequence: Synthetic peptide
TGGGGSGGGGS

SEQ ID NO: 37 - protein, 16 aa, Artificial Sequence
  Description of Artificial Sequence: Synthetic peptide
TGGGGSGGGGSGGGGS

SEQ ID NO: 38 - protein, 21 aa, Artificial Sequence
  Description of Artificial Sequence: Synthetic peptide
TGGGGSGGGGSGGGGSGGGGS

SEQ ID NO: 39 - protein, 26 aa, Artificial Sequence
  Description of Artificial Sequence: Synthetic peptide
TGGGGSGGGGSGGGGSGGGGSGGGGS

SEQ ID NO: 40 - protein, 31 aa, Artificial Sequence
  Description of Artificial Sequence: Synthetic polypeptide
TGGGGSGGGGSGGGGSGGGGSGGGGSGGGGS

SEQ ID NO: 41 - protein, 36 aa, Artificial Sequence
  Description of Artificial Sequence: Synthetic polypeptide
TGGGGSGGGGSGGGGSGGGGSGGGGSGGGGSGGGGS

SEQ ID NO: 42 - protein, 10 aa, Artificial Sequence
  Description of Artificial Sequence: Synthetic peptide
TGGGPKSCDK

SEQ ID NO: 43 - protein, 26 aa, Homo sapiens
MTSSLQRPWRVPWLPWTILLVSTAAA

SEQ ID NO: 44 - protein, 376 aa, Artificial Sequence
  Description of Artificial Sequence: Synthetic polypeptide
MDAMKRGLCCVLLLCGAVFVSPGASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - DNA, 1128 bp, Artificial Sequence
  Description of Artificial Sequence: Synthetic polynucleotide
atggatgcaatgaagagagggctctgctgtgtgctgctgctgtgtggagcagtcttcgtttcgcccggcgcctcgcagaatcaagaacgcctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 46 - protein, 352 aa, Artificial Sequence
  Description of Artificial Sequence: Synthetic polypeptide
SQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - DNA, 1056 bp, Artificial Sequence
  Description of Artificial Sequence: Synthetic polynucleotide
tcgcagaatcaagaacgcctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 48 - protein, 21 aa, Homo sapiens
MTLGSPRKGLLMLLMALVTQG

SEQ ID NO: 49 - protein, 352 aa, Artificial Sequence
  Description of Artificial Sequence: Synthetic polypeptide
MDAMKRGLCCVLLLCGAVFVSPGADPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 50 - protein, 328 aa, Artificial Sequence
  Description of Artificial Sequence: Synthetic polypeptide
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 25 aa, Homo sapiens
MDRGTLPLAVALLLASCSLSPTSLA

SEQ ID NO: 52 - protein, 820 aa, Artificial Sequence [sequence omitted]

SEQ ID NO: 53 - DNA, 2463 bp, Artificial Sequence
  Description of Artificial Sequence: Synthetic polynucleotide
atggatgcaatgaagagagggctctgctgtgtgctgctgctgtgtggagcagtcttcgtttcgcccggcgccgaaacagtccattgtgaccttcagcctgtgggccccgagagggacgaggtgacatataccactagccaggtctcgaagggctgcgtggctcaggcccccaatgccatccttgaagtccatgtcctcttcctggagttcccaacgggcccgtcacagctggagctgactctccaggcatccaagcaaaatggcacctggccccgagaggtgcttctggtcctcagtgtaaacagcagtgtcttcctgcatctccaggccctgggaatcccactgcacttggcctacaattccagcctggtcaccttccaagagcccccgggggtcaacaccacagagctgccatccttccccaagacccagatccttgagtgggcagctgagaggggccccatcacctctgctgctgagctgaatgacccccagagcatcctcctccgactgggccaagcccaggggtcactgtccttctgcatgctggaagccagccaggacatgggccgcacgctcgagtggcggccgcgtactccagccttggtccggggctgccacttggaaggcgtggccggccacaaggaggcgcacatcctgagggtcctgccgggccactcggccgggccccggacggtgacggtgaaggtggaactgagctgcgcacccggggatctcgatgccgtcctcatcctgcagggtcccccctacgtgtcctggctcatcgacgccaaccacaacatgcagatctggaccactggagaatactccttcaagatctttccagagaaaaacattcgtggcttcaagctcccagacacacctcaaggcctcctgggggaggcccggatgctcaatgccagcattgtggcatccttcgtggagctaccgctggccagcattgtctcacttcatgcctccagctgcggtggtaggctgcagacctcacccgcaccgatccagaccactcctcccaaggacacttgtagcccggagctgctcatgtccttgatccagacaaagtgtgccgacgacgccatgaccctggtactaaagaaagagcttgttgcgcatttgaagtgcaccatcacgggcctgaccttctgggaccccagctgtgaggcagaggacaggggtgacaagtttgtcttgcgcagtgcttactccagctgtggcatgcaggtgtcagcaagtatgatcagcaatgaggcggtggtcaatatcctgtcgagctcatcaccacagcggaaaaaggtgcactgcctcaacatggacagcctctctttccagctgggcctctacctcagcccacacttcctccaggcctccaacaccatcgagccggggcagcagagctttgtgcaggtcagagtgtccccatccgtctccgagttcctgctccagttagacagctgccacctggacttggggcctgagggaggcaccgtggaactcatccagggccgggcggccaagggcaactgtgtgagcctgctgtccccaagccccgagggtgacccgcgcttcagcttcctcctccacttctacacagtacccatacccaaaaccggcaccctcagctgcacggtagccctgcgtcccaagaccgggtctcaagaccaggaagtccataggactgtcttcatgcgcttgaacatcatcagccctgacctgtctggttgcacaagcaaaggcaccggtggtggacccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 54 - protein, 814 aa, Artificial Sequence
  Description of Artificial Sequence: Synthetic polypeptide
MDAMKRGLCCVLLLCGAVFVSPGAETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 21 aa, Homo sapiens
MGSLVLTLCALFCLAAYLVSG

SEQ ID NO: 56 - protein, 547 aa, Artificial Sequence
  Description of Artificial Sequence: Synthetic polypeptide
MDAMKRGLCCVLLLCGAVFVSPGASPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - DNA, 1644 bp, Artificial Sequence
  Description of Artificial Sequence: Synthetic polynucleotide
atggatgcaatgaagagagggctctgctgtgtgctgctgctgtgtggagcagtcttcgtttcgcccggcgccagccccatcatgaacctagagcagtctcctctggaagaagatatgtccctctttggtgatgttttctcagagcaagacggtgtcgactttaacacactgctccagagcatgaaggatgagtttcttaagacactaaacctctctgacatccccacgcaggattcagccaaggtggacccaccagagtacatgttggaactctacaacaaatttgcaacagatcggacctccatgccctctgccaacatcattaggagtttcaagaatgaagatctgttttcccagccggtcagttttaatgggctccgaaaataccccctcctcttcaatgtgtccattcctcaccatgaagaggtcatcatggctgaacttaggctatacacactggtgcaaagggatcgtatgatatacgatggagtagaccggaaaattaccatttttgaagtgctggagagcaaaggggataatgagggagaaagaaacatgctggtcttggtgtctggggagatatatggaaccaacagtgagtgggagacttttgatgtcacagatgccatcagacgttggcaaaagtcaggctcatccacccaccagctggaggtccacattgagagcaaacacgatgaagctgaggatgccagcagtggacggctagaaatagataccagtgcccagaataagcataaccctttgctcatcgtgttttctgatgaccaaagcagtgacaaggagaggaaggaggaactgaatgaaatgatttcccatgagcaacttccagagctggacaacttgggcctggatagcttttccagtggacctggggaagaggctttgttgcagatgagatcaaacatcatctatgactccactgcccgaatcagaaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 58 - protein, 523 aa, Artificial Sequence
  Description of Artificial Sequence: Synthetic polypeptide
SPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 544 aa, Artificial Sequence
  Description of Artificial Sequence: Synthetic polypeptide
MDAMKRGLCCVLLLCGAVFVSPGASPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - DNA, 1635 bp, Artificial Sequence
  Description of Artificial Sequence: Synthetic polynucleotide
atggatgcaatgaagagagggctctgctgtgtgctgctgctgtgtggagcagtcttcgtttcgcccggcgccagccccatcatgaacctagagcagtctcctctggaagaagatatgtccctctttggtgatgttttctcagagcaagacggtgtcgactttaacacactgctccagagcatgaaggatgagtttcttaagacactaaacctctctgacatccccacgcaggattcagccaaggtggacccaccagagtacatgttggaactctacaacaaatttgcaacagatcggacctccatgccctctgccaacatcattaggagtttcaagaatgaagatctgttttcccagccggtcagttttaatgggctccgaaaataccccctcctcttcaatgtgtccattcctcaccatgaagaggtcatcatggctgaacttaggctatacacactggtgcaaagggatcgtatgatatacgatggagtagaccggaaaattaccatttttgaagtgctggagagcaaaggggataatgagggagaaagaaacatgctggtcttggtgtctggggagatatatggaaccaacagtgagtgggagacttttgatgtcacagatgccatcagacgttggcaaaagtcaggctcatccacccaccagctggaggtccacattgagagcaaacacgatgaagctgaggatgccagcagtggacggctagaaatagataccagtgcccagaataagcataaccctttgctcatcgtgttttctgatgaccaaagcagtgacaaggagaggaaggaggaactgaatgaaatgatttcccatgagcaacttccagagctggacaacttgggcctggatagcttttccagtggacctggggaagaggctttgttgcagatgagatcaaacatcatctatgactccactgccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 61 - protein, 520 aa, Artificial Sequence
  Description of Artificial Sequence: Synthetic polypeptide
SPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 4 aa, Homo sapiens
RIRR

SEQ ID NO: 63 - protein, 6 aa, Artificial Sequence
  Description of Artificial Sequence: Synthetic 6xHis tag
HHHHHH

SEQ ID NO: 64 - protein, 10 aa, Artificial Sequence
  Description of Artificial Sequence: Synthetic peptide
  See specification as filed for detailed description of substitutions and preferred embodiments
GGGGSGGGGS

SEQ ID NO: 65 - protein, 225 aa, Homo sapiens; identical to SEQ ID NO: 23

SEQ ID NO: 66 - protein, 229 aa, Homo sapiens; identical to SEQ ID NO: 27

SEQ ID NO: 67 - protein, 223 aa, Homo sapiens; identical to SEQ ID NO: 24

SEQ ID NO: 68 - protein, 232 aa, Homo sapiens; identical to SEQ ID NO: 25

(19) United States
(12) Patent Application Publication
(10) Pub. No.: US 2023/0285506 A1
(43) Pub. Date: Sep. 14, 2023

(54) COMPOSITIONS AND METHODS FOR TREATING PULMONARY HYPERTENSION

(71) Applicant: Acceleron Pharma Inc., Cambridge, MA (US)

(72) Inventors: Gang Li, Cambridge, MA (US); Ravindra Kumar, Cambridge, MA (US)

(21) Appl. No.: 17/782,515

(22) PCT Filed: Dec. 3, 2020
(86) PCT No.: PCT/US2020/063160

§ 371 Date: Jun. 3, 2022

Related U.S. Application Data
(60) Provisional application No. 62/943,013, filed on Dec. 3, 2019.

(51) Int. Cl. A61K 38/17; A61P 9/12

(52) U.S. Cl. CPC A61K 38/179; A61P 9/12

(57) ABSTRACT

In some aspects, the disclosure relates to BMP antagonists, such as BMP 10 propeptides, and methods of using BMP antagonists to treat, prevent, or reduce the progression rate and/or severity of pulmonary hypertension (PH), particularly treating, preventing or reducing the progression rate and/or severity of one or more PH-associated complications. The disclosure also provides methods of using a BMP antagonist to treat, prevent, or reduce the progression rate and/or severity of a variety of conditions including, but not limited to, pulmonary vascular remodeling, pulmonary fibrosis, and right ventricular hypertrophy. The disclosure further provides methods of using a BMP antagonist to reduce right ventricular systolic pressure in a subject in need thereof.

### CROSS-REFERENCE TO RELATED APPLICATIONS

This application claims the benefit of and priority to U.S. Provisional Application No. 62/943,013, filed Dec. 3, 2019. The foregoing application is incorporated herein by reference in its entirety.

### BACKGROUND OF THE INVENTION

Pulmonary hypertension (PH) is a disease characterized by high blood pressure in lung vasculature, including pulmonary arteries, pulmonary veins, and pulmonary capillaries. In general, PH is defined as a mean pulmonary arterial (PA) pressure ≧25 mm Hg at rest or ≧30 mm Hg with exercise [Hill et al., Respiratory Care 54(7):958-68 (2009)]. The main PH symptom is difficulty in breathing or shortness of breath, and other symptoms include fatigue, dizziness, fainting, peripheral edema (swelling in foot, legs or ankles), bluish lips and skin, chest pain, angina pectoris, light-headedness during exercise, non-productive cough, racing pulse and palpitations. PH can be a severe disease causing heart failure, which is one of the most common causes of death in people who have pulmonary hypertension. Postoperative pulmonary hypertension may complicate many types of surgeries or procedures, and present a challenge associated with a high mortality.

PH may be grouped based on different manifestations of the disease sharing similarities in pathophysiologic mechanisms, clinical presentation, and therapeutic approaches [Simonneau et al., JACC 54(1):S44-54 (2009)]. Clinical classification of PH was first proposed in 1973, and a recent updated clinical classification was endorsed by the World Health Organization (WHO) in 2008. According to the updated PH clinical classification, there are five main groups of PH: pulmonary arterial hypertension (PAH), characterized by a PA wedge pressure ≦15 mm Hg; PH owing to a left heart disease (also known as pulmonary venous hypertension or congestive heart failure), characterized by a PA wedge pressure >15 mm Hg; PH owing to lung diseases and/or hypoxia; chronic thromboemboli PH; and PH with unclear or multifactorial etiologies [Simonneau et al., JACC 54(1):S44-54 (2009); Hill et al., Respiratory Care 54(7):958-68 (2009)]. PAH is further classified into idiopathic PAH (IPAH), a sporadic disease in which there is neither a family history of PAH nor an identified risk factor; heritable PAH; PAH induced by drugs and toxins; PAH associated with connective tissue diseases, HIV infection, portal hypertension, congenital heart diseases, schistosomiasis, and chronic hemolytic anemia; and persistent PH of newborns [Simonneau et al., JACC 54(1):S44-54 (2009)]. Diagnosis of various types of PH requires a series of tests.

In general, PH treatment depends on the cause or classification of the PH. Where PH is caused by a known medicine or medical condition, it is known as a secondary PH, and its treatment is usually directed at the underlying disease. Treatment of pulmonary venous hypertension generally involves optimizing left ventricular function by administering diuretics, beta blockers, and ACE inhibitors, or repairing or replacing a mitral valve or aortic valve. PAH therapies include pulmonary vasodilators, digoxin, diuretics, anticoagulants, and oxygen therapy. Pulmonary vasodilators target different pathways, including prostacyclin pathway (e.g., prostacyclins, including intravenous epoprostenol, subcutaneous or intravenous treprostinil, and inhaled iloprost), nitric oxide pathway (e.g., phosphodiesterase-5 inhibitors, including sildenafil and tadalafil), and endotheline-1 pathway (e.g., endothelin receptor antagonists, including oral bosentan and oral ambrisentan) [Humbert, M. Am. J. Respir. Crit. Care Med. 179:650-6 (2009); Hill et al., Respiratory Care 54(7):958-68 (2009)]. However, current therapies provide no cure for PH, and they do not directly treat the underling vascular remodeling and muscularization of blood vessels observed in many PH patients.

Thus, there is a high, unmet need for effective therapies for treating pulmonary hypertension. Accordingly, it is an object of the present disclosure to provide methods for treating, preventing, or reducing the progression rate and/or severity of PH, particular treating, preventing or reducing the progression rate and/or severity of one or more PH-associated complications.

### SUMMARY OF THE INVENTION

In part, the data presented herein demonstrates that BMP 10 antagonists (inhibitors) can be used to treat pulmonary hypertension. For example, it was shown that a soluble BMP 10 propeptide polypeptide can be used to reduce pulmonary arterial pressure and decrease right heart hypertrophy in a Sugen Hypoxia model of pulmonary arterial hypertension (PAH). Additionally, it was shown that a soluble BMP 10 propeptide polypeptide effectively improved right ventricular function (e.g., decreased right ventricular hypertrophy and reduced right ventricular free wall thickness), improved myocardial performance index, decreased right ventricular pressure, inhibited increases in right ventricular contractility, and reduced cardiac fibrosis in a pulmonary artery banding (PAB) model of PAH. Therefore, in some embodiments, the disclosure provides method for using various BMP signaling antagonists for treating hypertension, particularly pulmonary hypertension, including, for example, antagonists that inhibit one or more BMP ligands, particularly one or more of BMP10, BMP9, BMP6, BMP3b and BMP5; antagonists that inhibit one or more of BMPRII, ALK1, and endoglin; and antagonists that inhibit one or more downstream signaling components (e.g., Smad proteins). As used herein, such signaling antagonists are collectively referred to as “BMP antagonists” or “BMP inhibitors.” Accordingly, the disclosure provides in part, BMP antagonists compositions and methods for treating pulmonary hypertension (e.g., PAH), particularly treating one or more complications of pulmonary hypertension (e.g., elevated blood pressure, cardiac hypertrophy, vascular remodeling, and muscularization of vessels). BMP antagonists to be used in accordance with the methods and uses of the disclosure include, for example, ligand traps (e.g., soluble ALK1, BMPRII, BMP 10 propeptides, and endoglin polypeptides), antibody antagonists, small molecule antagonists, and polynucleotide antagonists. Optionally, BMP antagonists may be used in combination with one or more supportive therapies and/or additional active agents for treating pulmonary hypertension.

In certain aspects, the disclosure relates to methods of treating pulmonary hypertension comprising administering to a patient in need thereof an effective amount of a BMP antagonist. In some embodiments, the BMP antagonist is a BMP 10 antagonist. Effects on BMP 10 inhibition may be determined, for example, using a cell-based assay including those described herein (e.g., Smad signaling reporter assay). Such cell-based assays may be used to determine the inhibitory effects of other BMP antagonists including those described herein. Therefore, in some embodiments, a BMP 10 antagonist may bind to BMP 10. Ligand binding activity may be determined, for example, using a binding affinity assay including such as those described herein. Such ligand-binding assays may be used to determine the binding affinity of other BMP antagonists including those described herein. In some embodiments, a BMP10 antagonist binds to BMP10 with a K_{D} of at least 1 × 10⁻⁸ M (e.g., at least at least 1 × 10⁻⁹ M, at least 1 × 10⁻¹⁰ M, at least 1 × 10⁻¹¹ M, or at least 1 × 10⁻¹² M). In some embodiments, a BMP10 antagonist further inhibits the activity of BMP9. In some embodiments, the BMP 10 antagonist further inhibits one or more of BMP6, BMP3b, and BMP5. Therefore, in some embodiments, a BMP10 antagonist may bind to one or more of BMP9, BMP6, BMP3b, and BMP5. Examples of BMP10 antagonists are described herein and include, e.g., ligand traps (e.g., soluble, ligand-binding domain of BMPRII, ALK1, BMP 10 propeptide, and/or endoglin), antibodies, small molecules, and polynucleotides.

In certain aspects, a BMP antagonist to be used in accordance with methods and uses described herein is an agent that inhibits BMP9 (a BMP9 antagonist). Therefore, in some embodiments, a BMP9 antagonist may bind to BMP9. In some embodiments, a BMP9 antagonist binds to BMP9 with a K_{D} of at least 1 × 10⁻⁸ M (e.g., at least at least 1 × 10⁻⁹ M, at least 1 × 10⁻¹⁰ M, at least 1 × 10⁻¹¹ M, or at least 1 × 10⁻¹² M). In some embodiments, a BMP9 antagonist further inhibits the activity of BMP 10. In some embodiments, the BMP9 antagonist further inhibits one or more of BMP6, BMP3b, and BMP5. Therefore, in some embodiments, a BMP9 antagonist may bind to one or more of BMP10, BMP6, BMP3b, and BMP5. Examples of BMP9 antagonists are described herein and include, e.g., ligand traps (e.g., soluble, ligand-binding domain of BMPRII, ALK1, BMP10 propeptide, and/or endoglin, antibodies, small molecules, and polynucleotides.

In certain aspects, a BMP antagonist to be used in accordance with methods and uses described herein is an agent that inhibits BMP6 (a BMP6 antagonist). Therefore, in some embodiments, a BMP6 antagonist may bind to BMP6. In some embodiments, a BMP6 antagonist binds to BMP6 with a K_{D} of at least 1 × 10⁻⁸ M (e.g., at least at least 1 × 10⁻⁹ M, at least 1 × 10⁻¹⁰ M, at least 1 × 10⁻¹¹ M, or at least 1 × 10⁻¹² M). In some embodiments, a BMP6 antagonist further inhibits the activity of BMP 10 and/or BMP9. In some embodiments, the BMP6 antagonist further inhibits BMP3b and/or BMP5. Therefore, in some embodiments, a BMP6 antagonist may bind to one or more of BMP 10, BMP9, BMP3b, and BMP5. Examples of BMP6 antagonists are described herein and include, e.g., ligand traps (e.g., soluble, ligand-binding domain of BMPRII, ALK1, BMP10 propeptide, and/or endoglin), antibodies, small molecules, and polynucleotides.

In certain aspects, a BMP antagonist to be used in accordance with methods and uses described herein is an agent that inhibits BMP3b (a BMP3b antagonist). Therefore, in some embodiments, a BMP3b antagonist may bind to BMP3b. In some embodiments, a BMP3b antagonist binds to BMP3b with a KD of at least 1 × 10⁻⁸ M (e.g., at least at least 1 × 10⁻⁹ M, at least 1 × 10⁻¹⁰ M, at least 1 × 10⁻¹¹ M, or at least 1 × 10⁻¹² M). In some embodiments, a BMP3b antagonist further inhibits the activity of BMP 10 and/or BMP9. In some embodiments, the BMP3b antagonist further inhibits BMP6 and/or BMP5. Therefore, in some embodiments, a BMP3b antagonist may bind to one or more of BMP10, BMP9, BMP6, and BMP5. Examples of BMP3b antagonists are described herein and include, e.g., ligand traps (e.g., soluble, ligand-binding domain of BMPRII, ALK1, BMP10 propeptide, and/or endoglin), antibodies, small molecules, and polynucleotides.

In certain aspects, a BMP antagonist to be used in accordance with methods and uses described herein is an agent that inhibits BMP5 (a BMP5 antagonist). Therefore, in some embodiments, a BMP5 antagonist may bind to BMP5. In some embodiments, a BMP5 antagonist binds to BMP5 with a KD of at least 1 × 10⁻⁸ M (e.g., at least at least 1 × 10⁻⁹ M, at least 1 × 10⁻¹⁰ M, at least 1 × 10⁻¹¹ M, or at least 1 × 10⁻¹² M). In some embodiments, a BMP5 antagonist further inhibits the activity of BMP 10 and/or BMP9. In some embodiments, the BMP5 antagonist further inhibits BMP6 and/or BMP5. Therefore, in some embodiments, a BMP5 antagonist may bind to one or more of BMP10, BMP9, BMP6, and BMP3b. Examples of BMP5 antagonists are described herein and include, e.g., ligand traps (e.g., soluble, ligand-binding domain of BMPRII, ALK1, BMP10 propeptide, and/or endoglin), antibodies, small molecules, and polynucleotides.

In certain aspects, a BMP antagonist to be used in accordance with methods and uses described herein is an agent that inhibits one or more receptors or signaling mediators of one or more of BMP10, BMP9, BMP6, BMP3b, and BMP5. For example, in some embodiments, a BMP antagonist may inhibit BMPRII. In some embodiments, a BMP antagonist may inhibit ALK1. In some embodiments, a BMP antagonist may inhibit endoglin. In some embodiments, a BMP antagonist may inhibit one or more Smad proteins. Therefore, in some embodiments, a BMP antagonist may bind to one or more of BMPRII, endoglin, and Smad proteins. In some embodiments, a BMP antagonist binds to one or more of BMPRII, endoglin, and Smad proteins with a KD of at least 1 × 10⁻⁸ M (e.g., at least at least 1 × 10⁻⁹ M, at least 1 × 10⁻¹⁰ M, at least 1 × 10⁻¹¹ M, or at least 1 × 10⁻¹² M). Examples of BMPRII, endoglin, and Smad protein antagonists are described herein and include, e.g., antibodies, small molecules, and polynucleotides.

In some embodiments, the disclosure relates to a method of treating or reducing the progression rate and/or severity of one or more complications of pulmonary hypertension, comprising administering to a patient in need thereof an effective amount of a BMP 10 antagonist. In some embodiments, the one or more complications of pulmonary hypertension is selected from the group consisting of: smooth muscle and/or endothelial cell proliferation in the pulmonary artery, angiogenesis in the pulmonary artery, dyspnea, chest pain, pulmonary vascular remodeling, right ventricular hypertrophy, and pulmonary fibrosis. In some embodiments, the pulmonary hypertension is pulmonary arterial hypertension.

In some embodiments, the BMP 10 antagonist is an antibody or combination of antibodies. In some embodiments, the antibody or combination of antibodies binds to at least BMP 10. In some embodiments, the antibody is a multispecific antibody. In some embodiments, the multispecific antibody is a bi-specific antibody. In some embodiments, the BMP10 antagonist is a small molecule or combination of small molecules. In some embodiments, the small molecule or combination of small molecules inhibits at least BMP 10. In some embodiments, the BMP10 antagonist is a polynucleotide or combination of polynucleotides. In some embodiments, the polynucleotide or combination of polynucleotides inhibits at least BMP 10.

In some embodiments, the BMP 10 antagonist is a BMPRII polypeptide. The term BMPRII polypeptide collectively refers to naturally occurring polypeptides as well as truncations and variants thereof such as those described herein. Preferably BMPRII polypeptides comprise a ligand-binding domain of a BMPRII polypeptide or modified (variant) form thereof. For example, in some embodiments, a BMPRII polypeptide may comprise an extracellular domain of BMPRII. Preferably, BMPRII polypeptides to be used in accordance with the methods and uses described herein are soluble polypeptides. In some embodiments, the BMPRII polypeptide is a polypeptide comprising an amino acid sequence that is at least 70%, 75% 80%, 85%, 90%, 91%, 92%, 93%, 94%, 95%, 96%, 97%, 98%, 99%, or 100% identical to amino acids 27-150 of SEQ ID NO: 5. In some embodiments, the BMPRII polypeptide is a polypeptide comprising an amino acid sequence that is at least 70%, 75% 80%, 85%, 90%, 91%, 92%, 93%, 94%, 95%, 96%, 97%, 98%, 99%, or 100% identical to amino acids 34-123 of SEQ ID NO: 5. In some embodiments, the BMPRII polypeptide is a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 6. In some embodiments, a BMPRII polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 44. In some embodiments, a BMPRII polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 46.

In some embodiments, the BMP10 antagonist is an ALK1 polypeptide. The term ALK1 polypeptide collectively refers to naturally occurring polypeptides as well as truncations and variants thereof such as those described herein. Preferably ALK1 polypeptides comprise a ligand-binding domain of an ALK1 polypeptide or modified (variant) form thereof. For example, in some embodiments, an ALK1 polypeptide may comprise an extracellular domain of ALK1. Preferably, ALK1 polypeptides to be used in accordance with the methods and uses described herein are soluble polypeptides. In some embodiments, the ALK1 polypeptide is a polypeptide comprising an amino acid sequence that is at least 70%, 75% 80%, 85%, 90%, 91%, 92%, 93%, 94%, 95%, 96%, 97%, 98%, 99%, or 100% identical to amino acids 22-118 of SEQ ID NO: 11. In some embodiments, the ALK1 polypeptide is a polypeptide comprising an amino acid sequence that is at least 70%, 75% 80%, 85%, 90%, 91%, 92%, 93%, 94%, 95%, 96%, 97%, 98%, 99%, or 100% identical to amino acids 34-95 of SEQ ID NO: 11. In some embodiments, the ALK1 polypeptide is a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 12. In some embodiments, an ALK1 polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 49. In some embodiments, an ALK1 polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 50.

In some embodiments, the BMP 10 antagonist is an endoglin polypeptide. The term endoglin polypeptide collectively refers to naturally occurring polypeptides as well as truncations and variants thereof such as those described herein. Preferably endoglin polypeptides comprise a ligand-binding domain of an endoglin polypeptide or modified (variant) form thereof. For example, in some embodiments, an endoglin polypeptide may comprise an extracellular domain of endoglin. Preferably, endoglin polypeptides to be used in accordance with the methods and uses described herein are soluble polypeptides. In some embodiments, the endoglin polypeptide is a polypeptide comprising an amino acid sequence that is at least 70%, 75% 80%, 85%, 90%, 91%, 92%, 93%, 94%, 95%, 96%, 97%, 98%, 99%, or 100% identical to amino acids 26-378 of SEQ ID NO: 15. In some embodiments, the endoglin polypeptide is a polypeptide comprising an amino acid sequence that is at least 70%, 75% 80%, 85%, 90%, 91%, 92%, 93%, 94%, 95%, 96%, 97%, 98%, 99%, or 100% identical to amino acids 42-333 of SEQ ID NO: 15. In some embodiments, the endoglin polypeptide is a polypeptide comprising an amino acid sequence that is at least 70%, 75% 80%, 85%, 90%, 91%, 92%, 93%, 94%, 95%, 96%, 97%, 98%, 99%, or 100% identical to amino acids 26-346 of SEQ ID NO: 15. In some embodiments, the endoglin polypeptide is a polypeptide comprising an amino acid sequence that is at least 70%, 75% 80%, 85%, 90%, 91%, 92%, 93%, 94%, 95%, 96%, 97%, 98%, 99%, or 100% identical to amino acids 27-359 of SEQ ID NO: 15. In some embodiments, the endoglin polypeptide is a polypeptide comprising an amino acid sequence that is at least 70%, 75% 80%, 85%, 90%, 91%, 92%, 93%, 94%, 95%, 96%, 97%, 98%, 99%, or 100% identical to amino acids 26-359 of SEQ ID NO: 15. In some embodiments, the endoglin polypeptide does not comprise a sequence consisting of amino acids 379-430 of SEQ ID NO: 15. In some embodiments, the endoglin polypeptide does not comprise more than 50 consecutive amino acids from a sequence consisting of amino acids 379-586 of SEQ ID NO: 15. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 52. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 54. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 19. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 20. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 21. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 22.

In some embodiments, the BMP 10 antagonist is a BMP 10 propeptide polypeptide. The term BMP 10 propeptide polypeptide collectively refers to naturally occurring propeptide polypeptides as well as truncations and variants thereof such as those described herein. Preferably BMP10pro polypeptides comprise a ligand-binding domain of a BMP10 propeptide polypeptide or modified (variant) form thereof. Preferably, BMP10pro polypeptides to be used in accordance with the methods and uses described herein are soluble polypeptides. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 3 and ends at a position corresponding any one of amino acids 291-295 of SEQ ID NO: 3. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 3 and ends at a position corresponding any one of amino acids 291-294 of SEQ ID NO: 3, wherein the polypeptide does not comprise the sequence of amino acids RIRR. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 3. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 2-292 of SEQ ID NO: 3. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 3, wherein the polypeptide does not comprise the sequence of amino acids RIRR. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 2-292 of SEQ ID NO: 3, wherein the polypeptide does not comprise the sequence of amino acids RIRR. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 3. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 2-295 of SEQ ID NO: 3. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 3, wherein the polypeptide does not comprise the sequence of amino acids RIRR. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 2-295 of SEQ ID NO: 3, wherein the polypeptide does not comprise the sequence of amino acids RIRR. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 3 and ends at a position corresponding any one of amino acids 292-295 of SEQ ID NO: 3, wherein the C-terminus of the polypeptide is not R295 of SEQ ID NO: 3. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 3 and ends at a position corresponding any one of amino acids 292-296 of SEQ ID NO: 3, wherein the C-terminus of the polypeptide is not R296 of SEQ ID NO: 3. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 3, wherein the C-terminus of the polypeptide is not R295 of SEQ ID NO: 3. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 2-292 of SEQ ID NO: 3, wherein the C-terminus of the polypeptide is not R295 of SEQ ID NO: 3. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 3, wherein the C-terminus of the polypeptide is not R295 of SEQ ID NO: 3. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 2-295 of SEQ ID NO: 3, wherein the C-terminus of the polypeptide is not R295 of SEQ ID NO: 3. In some embodiments, a BMP10pro polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 56. In some embodiments, a BMP10pro polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 58. In some embodiments, a BMP10pro polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 59. In some embodiments, a BMP10pro polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 61. In some embodiments, the BMP10pro polypeptide is a polypeptide comprising an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 3 and ends at a position corresponding any one of amino acids 292-296 of SEQ ID NO: 3. In some embodiments, the BMP10pro polypeptide is a polypeptide comprising an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 3 and ends at a position corresponding any one of amino acids 292-295 of SEQ ID NO: 3, wherein the polypeptide does not comprise the sequence of amino acids RIRR. In some embodiments, the BMP10pro polypeptide is a polypeptide comprising an amino acid sequence that is at least 70%, 75%, 80%, 85%, 90%, 91%, 92%, 93%, 94%, 95%, 96%, 97%, 98%, 99%, or 100% identical to amino acids 2-292 of SEQ ID NO: 3. In some embodiments, the BMP10pro polypeptide is a polypeptide comprising an amino acid sequence that is at least 70%, 75%, 80%, 85%, 90%, 91%, 92%, 93%, 94%, 95%, 96%, 97%, 98%, 99%, or 100% identical to amino acids 2-292 of SEQ ID NO: 3, wherein the polypeptide does not comprise the sequence of amino acids RIRR. In some embodiments, the BMP10pro polypeptide is a polypeptide comprising an amino acid sequence that is at least 70%, 75%, 80%, 85%, 90%, 91%, 92%, 93%, 94%, 95%, 96%, 97%, 98%, 99%, or 100% identical to amino acids 2-295 of SEQ ID NO: 3. In some embodiments, the BMP10pro polypeptide is a polypeptide comprising an amino acid sequence that is at least 70%, 75%, 80%, 85%, 90%, 91%, 92%, 93%, 94%, 95%, 96%, 97%, 98%, 99%, or 100% identical to amino acids 2-295 of SEQ ID NO: 3, wherein the polypeptide does not comprise the sequence of amino acids RIRR. In some embodiments, the BMP10pro polypeptide is a polypeptide comprising an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 3 and ends at a position corresponding any one of amino acids 292-295 of SEQ ID NO: 3, wherein the C-terminus of the polypeptide is not R296 of SEQ ID NO: 3. In some embodiments, the BMP10pro polypeptide is a polypeptide comprising an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 3 and ends at a position corresponding any one of amino acids 292-295 of SEQ ID NO: 3, wherein the C-terminus of the polypeptide is not R296 of SEQ ID NO: 3. In some embodiments, the BMP10pro polypeptide is a polypeptide comprising an amino acid sequence that is at least 70%, 75%, 80%, 85%, 90%, 91%, 92%, 93%, 94%, 95%, 96%, 97%, 98%, 99%, or 100% identical to amino acids 2-292 of SEQ ID NO: 3, wherein the C-terminus of the polypeptide is not R296 of SEQ ID NO: 3. In some embodiments, the BMP10pro polypeptide is a polypeptide comprising an amino acid sequence that is at least 70%, 75%, 80%, 85%, 90%, 91%, 92%, 93%, 94%, 95%, 96%, 97%, 98%, 99%, or 100% identical to amino acids 2-295 of SEQ ID NO: 3, wherein the C-terminus of the polypeptide is not R296 of SEQ ID NO: 3. In some embodiments, the BMPRII, ALK1, endoglin, or BMP10pro polypeptide is a fusion protein comprising an immunoglobulin Fc domain. In some embodiments, the immunoglobulin Fc domain is an IgG1 Fc immunoglobulin domain. In some embodiments, the fusion protein comprises a linker domain positioned between the BMPRII, ALK1, endoglin, or BMP10pro polypeptide domain and the Fc immunoglobulin domain.

In certain aspects, BMP10pro polypeptides, BMPRII polypeptides, ALK1 polypeptides, and endoglin polypeptides, including variants thereof, may be fusion proteins. For example, in some embodiments, a BMP10pro polypeptide, BMPRII polypeptide, ALK1 polypeptide, or endoglin polypeptide may be a fusion protein comprising a BMP10pro polypeptide, BMPRII polypeptide, ALK1 polypeptide, or endoglin polypeptide domain and one or more heterologous (non-BMP10pro, non-BMPRII, non-ALK1, or non-endoglin) polypeptide domains. In some embodiments, a BMP10pro polypeptide, BMPRII polypeptide, ALK1 polypeptide, or endoglin polypeptide may be a fusion protein that has, as one domain, an amino acid sequence derived from a BMP10pro polypeptide, BMPRII polypeptide, ALK1 polypeptide, or endoglin polypeptide (e.g., a ligand-binding domain of a BMP propeptide, BMPRII receptor, ALK1 receptor, or endoglin receptor or a variant thereof) and one or more heterologous domains that provide a desirable property, such as improved pharmacokinetics, easier purification, targeting to particular tissues, etc. For example, a domain of a fusion protein may enhance one or more of in vivo stability, in vivo half-life, uptake/administration, tissue localization or distribution, formation of polypeptides, multimerization of the fusion protein, and/or purification. Optionally, a BMP10pro polypeptide, BMPRII polypeptide, ALK1 polypeptide, or endoglin polypeptide domain of a fusion protein is connected directly (fused) to one or more heterologous polypeptide domains, or an intervening sequence, such as a linker, may be positioned between the amino acid sequence of the BMP10pro polypeptide, BMPRII polypeptide, ALK1 polypeptide, or endoglin polypeptide and the amino acid sequence of the one or more heterologous domains. In certain embodiments, a BMP10pro, BMPRII, ALK1, or endoglin polypeptide fusion comprises a linker positioned between the heterologous domain and the BMP10pro domain, BMPRII domain, ALK1 domain, or endoglin domain. The linker may correspond to the roughly 4-15 amino acid unstructured region at the C-terminal end of the BMP10pro domain, BMPRII domain, ALK1 domain, or endoglin domain, or it may be an artificial sequence of between 3 and 15, 20, 30, 50 or more amino acids that are relatively free of secondary structure. A linker may be rich in glycine and proline residues and may, for example, contain repeating sequences of threonine/serine and glycines. Examples of linkers include, but are not limited to, the sequences TGGG (SEQ ID NO: 32), SGGG (SEQ ID NO: 33), TGGGG (SEQ ID NO: 30), SGGGG (SEQ ID NO: 31), GGGGS (SEQ ID NO: 34), GGGG (SEQ ID NO: 29), GGG (SEQ ID NO: 28), or a sequence selected from SEQ ID NOs: 35-42. In some embodiments, BMP10pro, BMPRII, ALK1, and endoglin fusion proteins may comprise a constant domain of an immunoglobulin, including, for example, the Fc portion of an immunoglobulin. For example, an amino acid sequence that is derived from an Fc domain of an IgG (IgG1, IgG2, IgG3, or IgG4), IgA (IgA1 or IgA2), IgE, or IgM immunoglobulin. For example, am Fc portion of an immunoglobulin domain may comprise, consist essentially of, or consist of an amino acid sequence that is at least 70%, 75%, 80%, 85%, 90%, 91%, 92%, 93%, 94%, 95%, 96%, 97%, 98%, 99% or 100% identical to any one of SEQ ID NOs: 23-27. Such immunoglobulin domains may comprise one or more amino acid modifications (e.g., deletions, additions, and/or substitutions) that confer an altered Fc activity, e.g., decrease of one or more Fc effector functions. In some embodiments, a BMP10pro, BMPRII, ALK1, or endoglin fusion protein comprises an amino acid sequence as set forth in the formula A-B-C. For example, the B portion is an N- and C-terminally truncated BMP10pro polypeptide as described herein. The A and C portions may be independently zero, one, or more than one amino acids, and both A and C portions are heterologous to B. The A and/or C portions may be attached to the B portion via a linker sequence. In certain embodiments, a BMP10pro, BMPRII, ALK1, or endoglin fusion protein comprises a leader sequence. The leader sequence may be a native BMP10pro, BMPRII, ALK1, or endoglin leader sequence or a heterologous leader sequence. In certain embodiments, the leader sequence is a tissue plasminogen activator (TPA) leader sequence.

A BMP10pro, BMPRII, ALK1, or endoglin polypeptide, including variants thereof, may comprise a purification subsequence, such as an epitope tag, a FLAG tag, a polyhistidine sequence, and a GST fusion. Optionally, a BMP10pro, BMPRII, ALK1, or endoglin polypeptide includes one or more modified amino acid residues selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent. BMP10pro, BMPRII, ALK1, and endoglin polypeptides may comprise at least one N-linked sugar, and may include two, three or more N-linked sugars. Such polypeptides may also comprise O-linked sugars. In general, it is preferable that BMP10pro, BMPRII, ALK1, and endoglin polypeptides be expressed in a mammalian cell line that mediates suitably natural glycosylation of the polypeptide so as to diminish the likelihood of an unfavorable immune response in a patient. BMP10pro, BMPRII, ALK1, and endoglin polypeptides may be produced in a variety of cell lines that glycosylate the protein in a manner that is suitable for patient use, including engineered insect or yeast cells, and mammalian cells such as COS cells, CHO cells, HEK cells and NSO cells. In some embodiments, a BMP10pro, BMPRII, ALK1, or endoglin polypeptide is glycosylated and has a glycosylation pattern obtainable from a Chinese hamster ovary cell line. In some embodiments, BMP10pro, BMPRII, ALK1, and endoglin polypeptides of the disclosure exhibit a serum half-life of at least 4, 6, 12, 24, 36, 48, or 72 hours in a mammal (e.g., a mouse or a human). Optionally, BMP10pro, BMPRII, ALK1, and endoglin polypeptides may exhibit a serum half-life of at least 6, 8, 10, 12, 14, 20, 25, or 30 days in a mammal (e.g., a mouse or a human).

In certain aspects, a BMP antagonist to be used in accordance with the teachings of the disclosure is an antibody or combination of antibodies. In some embodiments, the antibody is a multispecific antibody (e.g., a bispecific antibody) that binds to multiple different epitopes. In some embodiments, the combination of antibodies binds to multiple different epitopes. In some embodiments, the multiple different epitopes are epitopes on more than one different protein (e.g., any one or more of BMP10, BMP9, BMP6, BMP3b, BMP5, BMPRII, ALK1, or endoglin). In some embodiments, the antibody or combination of antibodies binds to at least BMP 10. In some embodiments, the antibody or combination of antibodies that binds to BMP 10 further binds to one or more of BMP9, BMP6, BMP3b, BMP5, BMPRII, ALK1, and endoglin. In some embodiments, the antibody or combination of antibodies binds to at least BMP9. In some embodiments, the antibody or combination of antibodies that binds to BMP9 further binds to one or more of BMP10, BMP6, BMP3b, BMP5, BMPRII, ALK1, and endoglin. In some embodiments, the antibody or combination of antibodies binds to at least BMP6. In some embodiments, the antibody or combination of antibodies that binds to BMP6 further binds to one or more of BMP9, BMP 10, BMP3b, BMP5, BMPRII, ALK1, and endoglin. In some embodiments, the antibody or combination of antibodies binds to at least BMP3b. In some embodiments, the antibody or combination of antibodies that binds to BMP 10 further binds to one or more of BMP9, BMP6, BMP 10, BMP5, BMPRII, ALK1, and endoglin. In some embodiments, the antibody or combination of antibodies binds to at least BMP5. In some embodiments, the antibody or combination of antibodies that binds to BMP5 further binds to one or more of BMP9, BMP6, BMP3b, BMP 10, BMPRII, ALK1, and endoglin. In some embodiments, the antibody or combination of antibodies binds to at least BMPRII. In some embodiments, the antibody or combination of antibodies that binds to BMPRII further binds to one or more of BMP10, BMP9, BMP6, BMP3b, BMP5, ALK1, and endoglin. In some embodiments, the antibody or combination of antibodies binds to at least ALK1. In some embodiments, the antibody or combination of antibodies that binds to ALK1 further binds to one or more of BMP10, BMP9, BMP6, BMP3b, BMP5, BMPRII, and endoglin. In some embodiments, the antibody or combination of antibodies binds to at least endoglin. In some embodiments, the antibody or combination of antibodies that binds to endoglin further binds to one or more of BMP10, BMP9, BMP6, BMP3b, BMP5, BMPRII, and ALK1. In some embodiments, the antibody or combination of antibodies binds to at least BMP 10 and BMP9. In some embodiments, the antibody or combination of antibodies that binds to BMP 10 and BMP9 further binds to one or more of BMP6, BMP3b, BMP5, BMPRII, ALK1, and endoglin. In certain preferred embodiments, antibodies or combinations of antibodies disclosed herein inhibit activity of one or more of BMP10, BMP9, BMP6, BMP3b, BMP5, BMPRII, ALK1, and endoglin. In certain preferred embodiments, a BMP 10 antibody binds to the mature BMP 10 protein. In certain preferred embodiments, a BMP 10 antibody binds to the mature BMP 10 protein competitively with a BMP 10 propeptide.

In certain aspects, a BMP antagonist to be used in accordance with the teachings of the disclosure is a small molecule or combination of small molecules. In some embodiments, a small molecule or combination of small molecules inhibits at least BMP 10 activity. In some embodiments, a small molecule or combination of small molecules that inhibits BMP 10 activity further inhibits the activity of one or more of BMP9, BMP6, BMP3b, BMP5, BMPRII, ALK1, endoglin, and Smad proteins. In some embodiments, a small molecule or combination of small molecules inhibits at least BMP9 activity. In some embodiments, a small molecule or combination of small molecules that inhibits BMP9 activity further inhibits the activity of one or more of BMP10, BMP6, BMP3b, BMP5, BMPRII, ALK1, endoglin, and Smad proteins. In some embodiments, a small molecule or combination of small molecules inhibits at least BMP6 activity. In some embodiments, a small molecule or combination of small molecules that inhibits BMP6 activity further inhibits the activity of one or more of BMP10, BMP9, BMP3b, BMP5, BMPRII, ALK1, endoglin, and Smad proteins. In some embodiments, a small molecule or combination of small molecules inhibits at least BMP3b activity. In some embodiments, a small molecule or combination of small molecules that inhibits BMP3b activity further inhibits the activity of one or more of BMP10, BMP9, BMP6, BMP5, BMPRII, ALK1, endoglin, and Smad proteins. In some embodiments, a small molecule or combination of small molecules inhibits at least BMP5 activity. In some embodiments, a small molecule or combination of small molecules that inhibits BMP5 activity further inhibits the activity of one or more of BMP10, BMP9, BMP6, BMP3b, BMPRII, ALK1, endoglin, and Smad proteins. In some embodiments, a small molecule or combination of small molecules inhibits at least BMPRII activity. In some embodiments, a small molecule or combination of small molecules that inhibits BMPRII activity further inhibits the activity of one or more of BMP10, BMP9, BMP6, BMP3b, BMP5, ALK1, endoglin, and Smad proteins. In some embodiments, a small molecule or combination of small molecules inhibits at least ALK1 activity. In some embodiments, a small molecule or combination of small molecules that inhibits ALK1 activity further inhibits the activity of one or more of BMP10, BMP9, BMP6, BMP3b, BMP5, BMPRII, endoglin, and Smad proteins. In some embodiments, a small molecule or combination of small molecules inhibits at least endoglin activity. In some embodiments, a small molecule or combination of small molecules that inhibits endoglin activity further inhibits the activity of one or more of BMP10, BMP9, BMP6, BMP3b, BMP5, BMPRII, ALK1, and Smad proteins. In some embodiments, a small molecule or combination of small molecules inhibits at least one or more Smads activity. In some embodiments, a small molecule or combination of small molecules that inhibits one or more Smads activity further inhibits the activity of one or more of BMP10, BMP9, BMP6, BMP3b, BMP5, BMPRII, ALK1, and endoglin. In some embodiments, a small molecule or combination of small molecules inhibits at least BMP10 and BMP9 activity. In some embodiments, a small molecule or combination of small molecules that inhibits BMP 10 and BMP9 activity further inhibits the activity of one or more of BMP6, BMP3b, BMP5, BMPRII, ALK1, endoglin, and Smad proteins.

In certain aspects, a BMP antagonist to be used in accordance with the teachings of the disclosure is a polynucleotide or combination of polynucleotides. In some embodiments, a polynucleotide or combination of polynucleotides inhibits at least BMP 10 activity. In some embodiments, a polynucleotide or combination of polynucleotides that inhibits BMP 10 activity further inhibits the activity of one or more of BMP9, BMP6, BMP3b, BMP5, BMPRII, ALK1, endoglin, and Smad proteins. In some embodiments, a polynucleotide or combination of polynucleotides inhibits at least BMP9 activity. In some embodiments, a polynucleotide or combination of polynucleotides that inhibits BMP9 activity further inhibits the activity of one or more of BMP10, BMP6, BMP3b, BMP5, BMPRII, ALK1, endoglin, and Smad proteins. In some embodiments, a polynucleotide or combination of polynucleotides inhibits at least BMP6 activity. In some embodiments, a polynucleotide or combination of polynucleotides that inhibits BMP6 activity further inhibits the activity of one or more of BMP10, BMP9, BMP3b, BMP5, BMPRII, ALK1, endoglin, and Smad proteins. In some embodiments, a polynucleotide or combination of polynucleotides inhibits at least BMP3b activity. In some embodiments, a polynucleotide or combination of polynucleotides that inhibits BMP3b activity further inhibits the activity of one or more of BMP 10, BMP9, BMP6, BMP5, BMPRII, ALK1, endoglin, and Smad proteins. In some embodiments, a polynucleotide or combination of polynucleotides inhibits at least BMP5 activity. In some embodiments, a polynucleotide or combination of polynucleotides that inhibits BMP5 activity further inhibits the activity of one or more of BMP 10, BMP9, BMP6, BMP3b, BMPRII, ALK1, endoglin, and Smad proteins. In some embodiments, a polynucleotide or combination of polynucleotides inhibits at least BMPRII activity. In some embodiments, a polynucleotide or combination of polynucleotides that inhibits BMPRII activity further inhibits the activity of one or more of BMP10, BMP9, BMP6, BMP3b, BMP5, ALK1, endoglin, and Smad proteins. In some embodiments, a polynucleotide or combination of polynucleotides inhibits at least ALK1 activity. In some embodiments, a polynucleotide or combination of polynucleotides that inhibits ALK1 activity further inhibits the activity of one or more of BMP 10, BMP9, BMP6, BMP3b, BMP5, BMPRII, endoglin, and Smad proteins. In some embodiments, a polynucleotide or combination of polynucleotides inhibits at least endoglin activity. In some embodiments, a polynucleotide or combination of polynucleotides that inhibits endoglin activity further inhibits the activity of one or more of BMP 10, BMP9, BMP6, BMP3b, BMP5, BMPRII, ALK1, and Smad proteins. In some embodiments, a polynucleotide or combination of polynucleotides inhibits at least one or more Smads activity. In some embodiments, a polynucleotide or combination of polynucleotides that inhibits one or more Smads activity further inhibits the activity of one or more of BMP10, BMP9, BMP6, BMP3b, BMP5, BMPRII, ALK1, and endoglin. In some embodiments, a polynucleotide or combination of polynucleotides inhibits at least BMP 10 and BMP9 activity. In some embodiments, a polynucleotide or combination of polynucleotides that inhibits BMP 10 and BMP9 activity further inhibits the activity of one or more of BMP6, BMP3b, BMP5, BMPRII, ALK1, endoglin, and Smad proteins.

In certain aspects, the present disclosure provides BMP 10 propeptides. As demonstrated by the examples herein, representative BMP 10 propeptides have been generated that that bind to and antagonize activity of a mature BMP 10 polypeptide. It was further discovered that these representative BMP 10 propeptides bind to other BMP proteins, particularly BMP9, BMP6, and BMP3b and to a lesser extent BMP5. Therefore, in some embodiments, BMP propeptides may antagonize other members of the BMP family and therefore may be useful in the treatment of additional disorders or conditions associated with these other BMP proteins (e.g., BMP9-, BMP6, BMP3b, and BMP6-associated disorders or conditions). Moreover, a C-terminally truncated BMP 10 propeptide variant, which lacks the four C-terminal amino acids of the propeptide domain, was surprisingly found to have increased BMP 10 antagonizing activity compared to a longer length BMP 10 propeptide variant. Therefore, in some embodiments, BMP 10 propeptides can tolerate C-terminal truncations of 1, 2, 3, or 4 amino acids without losing BMP 10 antagonizing activity. In addition, in some embodiments, BMP 10 propeptide variants lacking the four C-terminal amino acids may have increased BMP 10 antagonizing activity and therefore be useful in certain experimental and clinical situations where such increased BMP10 antagonism is desirable. The disclosure further provides a nucleic acid sequence encoding BMP10 propeptides, pharmaceutical compositions and kits comprising BMP 10 propeptides and methods of manufacturing BMP 10 propeptides.

In some embodiments, the disclosure provides BMP10pro polypeptides that are fusion proteins comprising a BMP10pro polypeptide domain and one or more heterologous (non-BMP10pro polypeptide domains. For example, BMP10pro polypeptides are Fc fusion proteins comprising a BMP10pro polypeptide domain and an immunoglobulin Fc domain. Optionally, a BMP10pro polypeptide domain of a fusion protein is connected directly (fused) to one or more heterologous polypeptide domains. In some embodiments, an intervening sequence, such as a linker, may be positioned between the amino acid sequence of the BMP10pro polypeptide and the amino acid sequence of the one or more heterologous domains. In certain embodiments, a BMP10pro polypeptide fusion comprises a linker positioned between the heterologous domain and the BMP10pro domain. The linker may correspond to the roughly 4-15 amino acid unstructured region at the C-terminal end of the BMP10pro domain, or it may be an artificial sequence of between 3 and 15, 20, 30, 50 or more amino acids that are relatively free of secondary structure. A linker may be rich in glycine and proline residues and may, for example, contain repeating sequences of threonine/serine and glycines. Examples of linkers include, but are not limited to, the sequences TGGG (SEQ ID NO: 32), SGGG (SEQ ID NO: 33), TGGGG (SEQ ID NO: 30), SGGGG (SEQ ID NO: 31), GGGGS (SEQ ID NO: 34), GGGG (SEQ ID NO: 29), GGG (SEQ ID NO: 28), or a sequence selected from SEQ ID NOs: 35-42. In some embodiments, BMP10pro fusion proteins may comprise a constant domain of an immunoglobulin, including, for example, the Fc portion of an immunoglobulin. For example, an amino acid sequence that is derived from an Fc domain of an IgG (IgG1, IgG2, IgG3, or IgG4), IgA (IgA1 or IgA2), IgE, or IgM immunoglobulin. For example, an Fc portion of an immunoglobulin domain may comprise an amino acid sequence that is at least 70%, 75%, 80%, 85%, 90%, 91%, 92%, 93%, 94%, 95%, 96%, 97%, 98%, 99% or 100% identical to any one of SEQ ID NOs: 23-27. Such immunoglobulin domains may comprise one or more amino acid modifications (e.g., deletions, additions, and/or substitutions) that confer an altered Fc activity, e.g., decrease of one or more Fc effector functions. In some embodiments, a BMP 10pro fusion protein comprises an amino acid sequence as set forth in the formula A-B-C. For example, the B portion is an N- and C-terminally truncated BMP10pro polypeptide as described herein. The A and C portions may be independently zero, one, or more than one amino acids, and both A and C portions are heterologous to B. The A and/or C portions may be attached to the B portion via a linker sequence. In certain embodiments, a BMP10pro fusion protein comprises a leader sequence. The leader sequence may be a native BMP10pro leader sequence or a heterologous leader sequence. In certain embodiments, the leader sequence is a tissue plasminogen activator (TPA) leader sequence.

In certain aspects, the disclosure provides BMP10pro polypeptides that are Fc fusion proteins comprising a BMP10pro polypeptide domain and an immunoglobulin Fc domain. In certain aspects, the disclosure provides a BMP10pro-Fc fusion protein comprising an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 3 and ends at a position corresponding any one of amino acids 292-295 of SEQ ID NO: 3. In some embodiments, BMP10pro-Fc fusion proteins do not comprise the sequence of amino acids RIRR. In some embodiments, the C-terminus of a BMP10pro domain of a BMP10pro-Fc fusion protein is not R296 of SEQ ID NO: 3. In some embodiments, a BMP10pro-Fc fusion protein comprises an amino acid sequence that is at least 70%, 75%, 80%, 85%, 90%, 91%, 92%, 93%, 94%, 95%, 96%, 97%, 98%, 99%, or 100% identical to amino acids 2-292 of SEQ ID NO: 3. In some embodiments, a BMP10pro-Fc fusion protein comprises an amino acid sequence that is at least 70%, 75%, 80%, 85%, 90%, 91%, 92%, 93%, 94%, 95%, 96%, 97%, 98%, 99%, or 100% identical to amino acids 2-292 of SEQ ID NO: 3 wherein polypeptide does not comprise the sequence of amino acids RIRR. In some embodiments, a BMP10pro-Fc fusion protein comprises an amino acid sequence that is at least 70%, 75%, 80%, 85%, 90%, 91%, 92%, 93%, 94%, 95%, 96%, 97%, 98%, 99%, or 100% identical to amino acids 2-292 of SEQ ID NO: 3 wherein the C-terminus of the BMP10pro domain is not R296 of SEQ ID NO: 3. In some embodiments, a BMP10pro-Fc fusion protein comprises an amino acid sequence that is at least 70%, 75%, 80%, 85%, 90%, 91%, 92%, 93%, 94%, 95%, 96%, 97%, 98%, 99%, or 100% identical to amino acids 2-295 of SEQ ID NO: 3. In some embodiments, a BMP10pro-Fc fusion protein comprises an amino acid sequence that is at least 70%, 75%, 80%, 85%, 90%, 91%, 92%, 93%, 94%, 95%, 96%, 97%, 98%, 99%, or 100% identical to amino acids 2-295 of SEQ ID NO: 3 wherein the BMP10pro domain does not comprise the sequence of amino acids RIRR. In some embodiments, a BMP10pro-Fc fusion protein comprises an amino acid sequence that is at least 70%, 75%, 80%, 85%, 90%, 91%, 92%, 93%, 94%, 95%, 96%, 97%, 98%, 99%, or 100% identical to amino acids 2-295 of SEQ ID NO: 3, wherein the C-terminus of the BMP10pro domain is not R296 of SEQ ID NO: 3. In some embodiments, a BMP10pro-Fc fusion protein comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 56. In some embodiments, a BMP10pro-Fc fusion protein comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 56, wherein the fusion protein does not comprise the sequence of amino acids RIRR. In some embodiments, a BMP10pro-Fc fusion protein comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 56, wherein the C-terminus of the BMP10pro domain is not R296 of SEQ ID NO: 3. In some embodiments, a BMP10pro-Fc fusion protein comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 61. In some embodiments, a BMP10pro-Fc fusion protein comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 61, wherein the fusion protein does not comprise the sequence of amino acids RIRR. In some embodiments, a BMP10pro-Fc fusion protein comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 61 wherein the C-terminus of the BMP10pro domain is not R296 of SEQ ID NO: 3.

A BMP10pro polypeptide, including variants thereof, may comprise a purification subsequence, such as an epitope tag, a FLAG tag, a polyhistidine sequence, and a GST fusion. Optionally, a BMP10pro polypeptide includes one or more modified amino acid residues selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent. BMP10pro polypeptides may comprise at least one N-linked sugar, and may include two, three or more N-linked sugars. Such polypeptides may also comprise O-linked sugars. In some embodiments, BMP10pro polypeptides are expressed in a mammalian cell line that mediates suitably natural glycosylation of the polypeptide so as to diminish the likelihood of an unfavorable immune response in a patient. BMP10pro polypeptides may be produced in a variety of cell lines that glycosylate the protein in a manner that is suitable for patient use, including engineered insect or yeast cells, and mammalian cells such as COS cells, CHO cells, HEK cells and NSO cells. In some embodiments, a BMP10pro polypeptide is glycosylated and has a glycosylation pattern obtainable from a Chinese hamster ovary cell line. In some embodiments, BMP10pro polypeptides exhibit a serum half-life of at least 4, 6, 12, 24, 36, 48, or 72 hours in a mammal (e.g., a mouse or a human). Optionally, a BMP10pro polypeptide may exhibit a serum half-life of at least 6, 8, 10, 12, 14, 20, 25, or 30 days in a mammal (e.g., a mouse or a human).

In certain aspects, the disclosure provides nucleic acids encoding a BMP 10 propeptide that do not encode a complete, translatable mature portion of a BMP 10. An isolated and/or recombinant polynucleotide may comprise a coding sequence for a BMP 10 propeptide, such as described above. An isolated nucleic acid may include a sequence coding for a BMP 10 propeptide and a sequence that would code for part or all of a mature portion, but for a stop codon positioned within the mature portion or positioned between the propeptide and the mature portion. In some embodiments, the disclosure provides a nucleic acid sequence that is at least 70%, 75% 80%, 85%, 90%, 91%, 92%, 93%, 94%, 95%, 96%, 97%, 98%, 99%, or 100% identical to the nucleic acid sequence of SEQ ID NO: 57. In some embodiments, the disclosure provides a nucleic acid sequence that is at least 70%, 75% 80%, 85%, 90%, 91%, 92%, 93%, 94%, 95%, 96%, 97%, 98%, 99%, or 100% identical to the nucleic acid sequence of SEQ ID NO: 60. Nucleic acids disclosed herein may be operably linked to a promoter for expression, and the disclosure provides vectors comprising such polynucleotides as well as cells transformed with such polynucleotides. Preferably the cell is a mammalian cell such as a CHO cell.

In certain aspects, the disclosure provides methods for making a BMP 10 propeptide. Such a method may include expressing any of the propeptide encoding nucleic acids disclosed herein in a suitable cell, such as a Chinese hamster ovary (CHO) cell. Such a method may comprise culturing a cell under conditions suitable for expression of the propeptide wherein the cell comprises a BMP10 propeptide expression construct. The method may further comprise a step of recovering the propeptide expressed BMP10 propeptide. BMP10 propeptides may be recovered as crude, partially purified or highly purified fractions using any of the well-known techniques for obtaining protein from cell cultures.

In certain aspects, the disclosure provides a use of a BMP10 propeptide for making a medicament for treating pulmonary hypertension (e.g., PAH), particularly treating one or more complications of pulmonary hypertension (e.g., elevated blood pressure, cardiac hypertrophy, vascular remodeling, and muscularization of vessels) as well as for other cardiac-related uses described herein.

In certain aspects, the disclosure provides methods for identifying an agent that may be used for treating pulmonary hypertension (e.g., PAH) or one or more complications of pulmonary hypertension. A method may comprise: a) identifying a test agent that binds a mature BMP 10 polypeptide competitively with a BMP 10 propeptide; and b) evaluating the effect of the agent on pulmonary hypertension or the one or more complications of pulmonary hypertension. A test agent may be, for example, a variant BMP 10 propeptide, an antibody, or a small molecule.

In certain aspects, the disclosure provides pharmaceutical preparations (compositions) comprising a BMP10pro polypeptide and a pharmaceutically acceptable carrier. A pharmaceutical preparation comprising a BMP10pro polypeptide may also comprise one or more additional active agents such as a compound that is used to treat or prevent a disorder or condition as described herein [e.g., pulmonary hypertension or one or more complications of pulmonary hypertension]. In some embodiments, a pharmaceutical preparation comprising a BMP10pro polypeptide will be pyrogen-free (e.g., pyrogen free to the extent required by regulations governing the quality of products for therapeutic use).

In some embodiments, the method comprises further administering to the patient an additional active agent and/or supportive therapy for treating pulmonary hypertension. In some embodiments, the additional active agent and/or supportive therapy for treating pulmonary hypertension is selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1-->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1-->3)-beta-D-glucopyranosyl] oleanolic acid 28-O-beta-D-glucopyranosyl ester; 3-O-[beta-D-glucopyranosyl-(1-->2)-beta-D-glucopyranosyl] oleanolic acid 28-O-beta-D-glucopyranosyl ester; 3-O-[a-L-rhamnopyranosyl-(1-->3)-beta-D-glucuronopyranosyl] oleanolic acid; 3-O-[alpha-L-rhamnopyranosyl-(1-->3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1→3)β-D-glucopyranosiduronic acid (CS1); oleanolic acid 3-O-β-D-glucopyranosyl (1→73)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3ʹ,4ʹ:2,3]oleanolate); lung and/or heart transplantation.

In some embodiments, the patient has resting pulmonary arterial pressure (PAP) of at least 25 mm Hg (e.g., 25, 30, 35, 40, 45, or 50 mm Hg). In some embodiments, the method reduces PAP in the patient. In some embodiments, the method reduces PAP by at least 3 mmHg (e.g., at least 3, 5, 7, 10, 12, 15, 20, or 25 mm Hg) in the patient. In some embodiments, the method reduces pulmonary vascular resistance in the patient. In some embodiments, the method increases pulmonary capillary wedge pressure. In some embodiments, the method increases left ventricular end-diastolic pressure. In some embodiments, the method increases exercise capacity of the patient. In some embodiments, the method increases the patient’s 6-minute walk distance. In some embodiments, the method increases the patient’s 6-minute walk distance by at least 10 meters (e.g., at least 10, 20, 30, 40, 50, 60, 70, 80, 90, 100 or more meters). In some embodiments, the method reduces the patient’s Borg dyspnea index (BDI). In some embodiments, the method reduces the patient’s BDI by at least 0.5 index points (e.g., at least 0.5, 1, 1.5, 2, 2.5, 3, 3.5, 4, 4.5, 5, 5.5, 6, 6.5, 7, 7.5, 8, 8.5, 9, 9.5, or 10 index points). In some embodiments, the patient has Functional Class I, Class II, Class III, or Class IV pulmonary hypertension as recognized by the World Health Organization. In some embodiments, the method prevents or delays pulmonary hypertension Functional Class progression (e.g., prevents or delays progression from Functional Class I to Class II, Class II to Class III, or Class III to Class IV pulmonary hypertension as recognized by the World Health Organization). In some embodiments, the method promotes or increases pulmonary hypertension Functional Class regression (e.g., promotes or increases regression from Class IV to Class III, Class III to Class II, or Class II to Class I pulmonary hypertension as recognized by the World Health Organization).

### BRIEF DESCRIPTION OF THE DRAWINGS

FIG. 1 shows multiple sequence alignment of Fc domains from human IgG isotypes using Clustal 2.1. Hinge regions are indicated by dotted underline. Double underline indicates examples of positions engineered in IgG1 Fc to promote asymmetric chain pairing and the corresponding positions with respect to other isotypes IgG2, IgG3 and IgG4.

FIG. 2 shows the domain structure of hENG-Fc fusion constructs. Full-length ENG extracellular domain (residues 26-586 in top structure) consists of an orphan domain and N-terminal and C-terminal zona pellucida (ZP) domains. Below it are shown structures of selected truncated variants and whether they exhibit high-affinity binding (+/-) to BMP-9 and BMP-10 in an SPR-based assay.

FIG. 3 shows a nucleic acid sequence encoding a human BMP 10 precursor protein, designated as SEQ ID NO: 2.

FIG. 4 shows a nucleic acid sequence encoding a human BMP 10 propeptide domain protein, designated as SEQ ID NO: 4.

FIG. 5. Effects of BMP 10 propeptide (BMP10pro) were examined using the Sugen Hypoxia (Su/Hx)model of PAH. Rats were separated into four treatment groups: 1) control rats (Tris buffered saline administered s.c. as 1 ml/kg, every three days), “Normal”; 2) treatment with semaxanib (200 mg/kg administered s.c. as a single dose daily)/hypoxia and Tris buffered saline (administered s.c. as 1 ml/kg, every three days) (vehicle treatment group), “PBS”; 3) treatment with BMP10pro (10 mg/kg administered s.c. every three days) and semaxanib (200 mg/kg administered s.c. as a single dose daily)/hypoxia, “BMP10pro”; and 4) treatment with sildenafil (30 mg/kg administered orally twice daily) and semaxanib (200 mg/kg administered s.c. as a single dose daily)/hypoxia, “Sildenafil”. (FIG. 5A) shows systolic pulmonary artery pressure (sPAP) and (FIG. 5B) shows calculated mean pulmonary artery pressure (mPAP). Hypertrophy was assessed, in part, by measuring the weight of and calculating right ventricle (RV)/left ventricle (LV) + septum (S) (FIG. 5C). Compared to “Normal” animals, “PBS” animals were observed to have elevated PAP and right heart hypertrophy. Sildenafil treated animals, “Sildenafil”, had a mean pulmonary arterial pressure that was reduced by 24.8% and right heart hypertrophy was decreased by 3.8% compared to “PBS” animals. BMP10pro treatment (“BMP10pro” animals) resulted in a reduction of mean pulmonary arterial pressure by 34% and decreased right heart hypertrophy by 19.4% compared to “PBS” animals, indicating that BMP 10pro treatment was found have greater effects in treating PAH in this model compared to sildenafil. Statistical significance (p value) is depicted as *: p<0.05; **: p<0.01; ***: p<0.001; and ****: p<0.0001.

FIG. 6. The effects of BMP 10 propeptide (BMP10pro) were examined in the pulmonary artery banding (PAB) model of right ventricle (RV) failure in mice. Mice were separated into two PAB treatment groups: 1) treatment with Tris buffered saline (administered s.c. as 1 ml/kg, every three days) (vehicle treatment group, “PAB-PBS”), 2) treatment with BMP10pro (10 mg/kg administered s.c. every three days), “PAB-BMP10pro”, plus 3) a sham group, “Sham”. Echocardiography was performed to measure RV free wall thickness (RVFWT) (FIG. 6A) and decreased tricuspid annular plane systolic excursion (TAPSE) (FIG. 6B), and RV global function was estimated by myocardial performance index (MPI) (FIG. 6C). Right ventricular developed pressure (RVDP) (FIG. 6D) and maximal +dp/dt and -dp/dt (FIG. 6E) were calculated. Right heart Hypertrophy was assessed by calculating RV/LV + S (FIG. 6F). RV pressure (RVDP) in PAB treated mice was significantly increased on day 21 in “PAB-PBS” animals and BMP10pro treatment decreased RVDP by 24.4% after 3 wks-treatment in “PAB-BMP10pro” animals (FIG. 6D). Similar to RVDP, RV contractility measured by +dp/dt and -dp/dt, was significantly increased in “PAB-PBS” animals, which was significantly inhibited by BMP10pro treatment in “PAB-BMP10pro” animals (FIG. 6E). In addition, PAB induced cardiac fibrosis and 19.5% of fibrosis area was observed in “PAB-PBS” animals, which was reduced to 10.3% in the “PAB-BMP10pro” treated group, suggesting the inhibition of RV remodeling by BMP10pro (FIG. 6G). Statistical significance (p value) is depicted as *: p<0.05; **: p<0.01; ***: p<0.001; and ****: p<0.0001.

### DETAILED DESCRIPTION OF THE INVENTION

### 1. Overview

The TGFβ superfamily is comprised of over 30 secreted factors including TGFβs, activins, nodals, bone morphogenetic proteins (BMPs), growth and differentiation factors (GDFs), and anti-Mullerian hormone (AMH) [Weiss et al. (2013) Developmental Biology, 2(1): 47-63]. Members of the superfamily, which are found in both vertebrates and invertebrates, are ubiquitously expressed in diverse tissues and function during the earliest stages of development throughout the lifetime of an animal. Indeed, TGFβ superfamily proteins are key mediators of stem cell self-renewal, gastrulation, differentiation, organ morphogenesis, and adult tissue homeostasis. Consistent with this ubiquitous activity, aberrant TGFβ superfamily signaling is associated with a wide range of human pathologies including, for example, autoimmune disease, cardiovascular disease, fibrotic disease, and cancer.

Ligands of the TGFβ superfamily share the same dimeric structure in which the central 3-½ turn helix of one monomer packs against the concave surface formed by the beta-strands of the other monomer. The majority of TGFβ family members are further stabilized by an intermolecular disulfide bond. This disulfide bonds traverses through a ring formed by two other disulfide bonds generating what has been termed a ‘cysteine knot’ motif [Lin et al. (2006) Reproduction 132: 179-190; and Hinck et al. (2012) FEBS Letters 586: 1860-1870].

TGFβ superfamily signaling is mediated by heteromeric complexes of type I and type II serine/threonine kinase receptors, which phosphorylate and activate downstream SMAD proteins (e.g., SMAD proteins 1, 2, 3, 5, and 8) upon ligand stimulation [Massague (2000) Nat. Rev. Mol. Cell Biol. 1: 169-178]. These type I and type II receptors are transmembrane proteins, composed of a ligand-binding extracellular domain with cysteine-rich region, a transmembrane domain, and a cytoplasmic domain with predicted serine/threonine kinase specificity. In general, type I receptors mediate intracellular signaling while the type II receptors are required for binding TGF-beta superfamily ligands. Type I and II receptors form a stable complex after ligand binding, resulting in phosphorylation of type I receptors by type II receptors.

The TGFβ family can be divided into two phylogenetic branches based on the type I receptors they bind and the Smad proteins they activate. One is the more recently evolved branch, which includes, e.g., the TGFβs, activins, GDF8, GDF9, GDF11, BMP3 and nodal, which signal through type I receptors that activate Smads 2 and 3 [Hinck (2012) FEBS Letters 586:1860-1870]. The other branch comprises the more distantly related proteins of the superfamily and includes, e.g., BMP2, BMP4, BMP5, BMP6, BMP7, BMP8a, BMP8b, BMP9, BMP10, GDF1, GDF5, GDF6, and GDF7, which signal through Smads 1, 5, and 8.

Activins are members of the TGFβ superfamily and were initially discovered as regulators of secretion of follicle-stimulating hormone, but subsequently various reproductive and non-reproductive roles have been characterized. There are three principal activin forms (A, B, and AB) that are homo/heterodimers of two closely related β subunits (β_{A}β_{A}, β_{B}β_{B}, and β_{A}β_{B}, respectively). The human genome also encodes an activin C and an activin E, which are primarily expressed in the liver, and heterodimeric forms containing β_{C} or β_{E} are also known. In the TGF-beta superfamily, activins are unique and multifunctional factors that can stimulate hormone production in ovarian and placental cells, support neuronal cell survival, influence cell-cycle progress positively or negatively depending on cell type, and induce mesodermal differentiation at least in amphibian embryos [DePaolo et al. (1991) Proc Soc Ep Biol Med. 198:500-512; Dyson et al. (1997) Curr Biol. 7:81-84; and Woodruff (1998) Biochem Pharmacol. 55:953-963]. In several tissues, activin signaling is antagonized by its related heterodimer, inhibin. For example, in the regulation of follicle-stimulating hormone (FSH) secretion from the pituitary, activin promotes FSH synthesis and secretion, while inhibin reduces FSH synthesis and secretion. Other proteins that may regulate activin bioactivity and/or bind to activin include follistatin (FS), follistatin-related protein (FSRP, also known as FLRG or FSTL3), and α₂-macroglobulin.

The BMPs and GDFs together form a family of cysteine-knot cytokines sharing the characteristic fold of the TGFβ superfamily [Rider et al. (2010) Biochem J., 429(1): 1-12]. This family includes, for example, BMP2, BMP4, BMP6, BMP7, BMP2a, BMP3, BMP3b (also known as GDF10), BMP4, BMP5, BMP6, BMP7, BMP8, BMP8a, BMP8b, BMP9 (also known as GDF2), BMP10, BMP11 (also known as GDF11), BMP12 (also known as GDF7), BMP13 (also known as GDF6), BMP14 (also known as GDF5), BMP15, GDF1, GDF3 (also known as VGR2), GDF8 (also known as myostatin), GDF9, GDF15, and decapentaplegic. Besides the ability to induce bone formation, which gave the BMPs their name, the BMP/GDFs display morphogenetic activities in the development of a wide range of tissues. BMP/GDF homo- and hetero-dimers interact with combinations of type I and type II receptor dimers to produce multiple possible signaling complexes, leading to the activation of one of two competing sets of SMAD transcription factors. BMP/GDFs have highly specific and localized functions. These are regulated in a number of ways, including the developmental restriction of BMP/GDF expression and through the secretion of several specific BMP antagonist proteins that bind with high affinity to the cytokines. Curiously, a number of these antagonists resemble TGFβ superfamily ligands.

Growth and differentiation factor-8 (GDF8) is also known as myostatin. GDF8 is a negative regulator of skeletal muscle mass and is highly expressed in developing and adult skeletal muscle. The GDF8 null mutation in transgenic mice is characterized by a marked hypertrophy and hyperplasia of skeletal muscle [McPherron et al. Nature (1997) 387:83-90]. Similar increases in skeletal muscle mass are evident in naturally occurring mutations of GDF8 in cattle and, strikingly, in humans [Ashmore et al. (1974) Growth, 38:501-507; Swatland and Kieffer, J. Anim. Sci. (1994) 38:752-757; McPherron and Lee, Proc. Natl. Acad. Sci. USA (1997) 94:12457-12461; Kambadur et al. Genome Res. (1997) 7:910-915; and Schuelke et al. (2004) N Engl J Med, 350:2682-8]. Studies have also shown that muscle wasting associated with HIV-infection in humans is accompanied by increases in GDF8 protein expression [Gonzalez-Cadavid etal., PNAS (1998) 95:14938-43]. In addition, GDF8 can modulate the production of muscle-specific enzymes (e.g., creatine kinase) and modulate myoblast cell proliferation [International Patent Application Publication No. WO 00/43781]. The GDF8 propeptide can noncovalently bind to the mature GDF8 domain dimer, inactivating its biological activity [Miyazono et al. (1988) J. Biol. Chem., 263: 6407-6415; Wakefield et al. (1988) J. Biol. Chem., 263; 7646-7654; and Brown et al. (1990) Growth Factors, 3: 35-43]. Other proteins which bind to GDF8 or structurally related proteins and inhibit their biological activity include follistatin, and potentially, follistatin-related proteins [Gamer et al. (1999) Dev. Biol., 208: 222-232].

GDF11, also known as BMP11, is a secreted protein that is expressed in the tail bud, limb bud, maxillary and mandibular arches, and dorsal root ganglia during mouse development [McPherron et al. (1999) Nat. Genet., 22: 260-264; and Nakashima et al. (1999) Mech. Dev., 80: 185-189]. GDF11 plays a unique role in patterning both mesodermal and neural tissues [Gamer et al. (1999) Dev Biol., 208:222-32]. GDF11 was shown to be a negative regulator of chondrogenesis and myogenesis in developing chick limb [Gamer et al. (2001) Dev Biol., 229:407-20]. The expression of GDF11 in muscle also suggests its role in regulating muscle growth in a similar way to GDF8. In addition, the expression of GDF11 in brain suggests that GDF11 may also possess activities that relate to the function of the nervous system. Interestingly, GDF11 was found to inhibit neurogenesis in the olfactory epithelium [Wu et al. (2003) Neuron., 37:197-207]. Hence, GDF11 may have in vitro and in vivo applications in the treatment of diseases such as muscle diseases and neurodegenerative diseases (e.g., amyotrophic lateral sclerosis).

As demonstrated herein, a soluble BMP10pro polypeptide, which binds to various BMP proteins including BMP 10, BMP9, BMP6, BMP3b, and BMP5, is effective in reducing pulmonary arterial pressure and decreasing right heart hypertrophy in a PAH model. Furthermore, a soluble BMP10pro polypeptide is effective in ameliorating various complications of PAH, such as reducing pulmonary artery pressure, inhibiting RV remodeling, and improving RV function in a PAH model. While not wishing to be bound to any particular mechanism or by any particular theory, it is expected that the effects of BMP10pro polypeptides are caused primarily by a BMP signaling antagonist effect, particularly of one or more of BMP 10, BMP9, BMP6, BMP3b, and BMP5. Regardless of the mechanism, it is apparent from the data presented herein that BMP signaling antagonists do have positive effects in treating pulmonary hypertension, such as pulmonary arterial hypertension.

The animal models for PAH that were used in the studies described herein are considered to be predicative of efficacy in humans, and therefore, this disclosure provides methods for using BMP10pro polypeptides, endoglin polypeptides, and other BMP antagonists to treat pulmonary hypertension (e.g., PAH), particularly treating, preventing, or reducing the severity or duration of one or more complications of pulmonary hypertension, in humans.

As disclosed herein, the term BMP antagonist refers a variety of agents that may be used to antagonize BMP signaling including, for example, antagonists that inhibit one or more BMP ligands [e.g., BMP 10, BMP9, BMP6, BMP3b, and BMP5]; antagonists that inhibit one or more BMP-interacting BMPRII, ALK1, and endoglin; and antagonists that inhibit one or more downstream signaling components (e.g., Smad proteins). BMP antagonists to be used in accordance with the methods and uses of the disclosure include a variety of forms, for example, ligand traps (e.g., soluble BMP10pro polypeptides, ALK1 polypeptides, and endoglin polypeptides), antibody antagonists (e.g., antibodies that inhibit one or more of BMP10, BMP9, BMP6, BMP3b, BMP5, ALK1, BMPRII, and endoglin), small molecule antagonists [e.g., small molecules that inhibit one or more of BMP10, BMP9, BMP6, BMP3b, BMP5, ALK1, BMPRII, endoglin, and one or more Smad proteins], and polynucleotide antagonists [e.g., polynucleotide sequences that inhibit one or more of BMP 10, BMP9, BMP6, BMP3b, BMP5, ALK1, BMPRII, endoglin, and one or more Smad proteins].

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein’s gene expression or by inducing an inactive protein to enter an active state) or increasing a protein’s and/or gene’s activity.

“Antagonize”, in all its grammatical forms, refers to the process of inhibiting a protein and/or gene (e.g., by inhibiting or decreasing that protein’s gene expression or by inducing an active protein to enter an inactive state) or decreasing a protein’s and/or gene’s activity.

The terms “about” and “approximately” as used in connection with a numerical value throughout the specification and the claims denotes an interval of accuracy, familiar and acceptable to a person skilled in the art. In general, such interval of accuracy is ± 10%. Alternatively, and particularly in biological systems, the terms “about” and “approximately” may mean values that are within an order of magnitude, preferably ≤ 5 -fold and more preferably ≤ 2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers.

### 2. BMP10 Propeptide Polypeptides, ALK1 Polypeptides, BMPRII Polypeptides, and Endoglin Polypeptides

In certain aspects, the disclosure relates to BMP 10 propeptide (BMP10pro) polypeptides and uses thereof (e.g., treating, preventing, or reducing the progression rate and/or severity of pulmonary hypertension (e.g., pulmonary arterial hypertension) or one or more complications of pulmonary hypertension and/or interstitial lung disease (e.g., idiopathic pulmonary fibrosis)). As used herein, the term “BMP10 polypeptide” refers to the family of bone morphogenetic proteins of the type 10 derived from any species. The term “BMP10 polypeptide” includes any of the naturally occurring BMP 10 polypeptides as well as any variants thereof (including mutants, fragments, fusions, and peptidomimetic forms) that retain a useful activity. A naturally occurring BMP10 protein is generally encoded as a larger precursor that typically contains a signal sequence at its N-terminus followed by a dibasic amino acid cleavage site and a propeptide, followed by another dibasic amino acid cleavage site and a mature domain. The human BMP10 precursor sequence (NCBI NP_055297) is shown below:

1 MGSLVLTLCA LFCLAAYLVS GSPIMNLEQS PLEEDMSLFG DVFSEQDGVD
51 FNTLLQSMKD EFLKTLNLSD IPTQDSAKVD PPEYMLELYN KFATDRTSMP
101 SANIIRSFKN EDLFSQPVSF NGLRKYPLLF NVSIPHHEEV IMAELRLYTL
151 VQRDRMIYDG VDRKITIFEV LESKGDNEGE RNMLVLVSGE IYGTNSEWET
201 FDVTDAIRRW QKSGSSTHQL EVHIESKHDE AEDASSGRLE IDTSAQNKHN
251 PLLIVFSDDQ SSDKERKEEL NEMISHEQLP ELDNLGLDSF SSGPGEEALL
301 QMRSNIIYDS TARIRRNAKG NYCKRTPLYI DFKEIGWDSW IIAPPGYEAY
351 ECRGVCNYPL AEHLTPTKHA IIQALVHLKN SQKASKACCV PTKLEPISIL
401 YLDKGVVTYK FKYEGMAVSE CGCR (SEQ ID NO: 1)

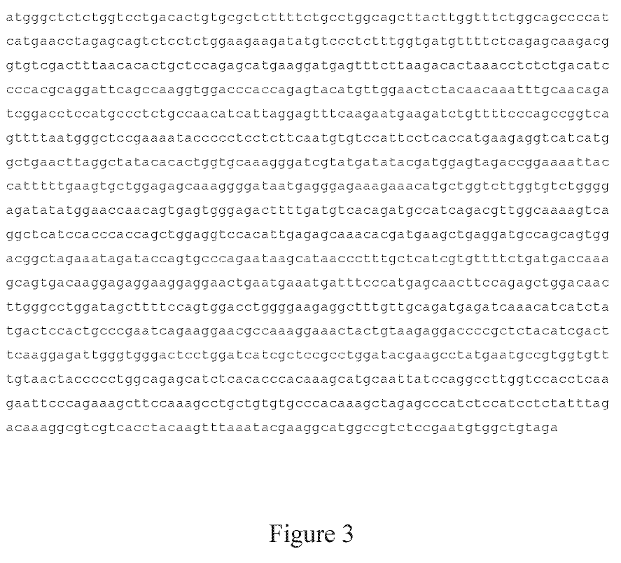

The signal peptide (amino acids 1-21) is underlined; the mature protein (amino acids 317-424) is double underlined; and potential N-linked glycosylation sites are boxed. FIG. 3 shows a nucleic acid sequence encoding the BMP 10 precursor protein of SEQ ID NO: 1 (this nucleic acid is designated SEQ ID NO: 2).

The term “BMP10 propeptide” or “BMP10pro” is used to refer to polypeptides comprising any naturally occurring propeptide of a BMP 10 family member as well as any variants thereof (including mutants, fragments and peptidomimetic forms) that retain a useful activity. Examples of useful activities of BMP10pro polypeptides include binding to the mature portion of a BMP 10 protein and acting as an antagonist of an activity of a mature BMP 10. As demonstrated herein, BMP10pro polypeptides may also bind to one or more of BMP9, BMP6, BMP3b, and BMP5. Thus, in some embodiments, BMP10pro polypeptides may be further used to as an antagonist of one or more of BMP9, BMP6, BMP3b, and BMP5. Functional variants of a BMP10 propeptide may be characterized by, for example, binding to mature BMP 10 protein and/or the ability to competitively inhibit the binding of BMP 10 to a receptor such as BMPRII, ALK1, and/or endoglin.

A human BMP10 propeptide sequence is shown below:

GSPIMNLEQS PLEEDMSLFG DVFSEQDGVD
FNTLLQSMKD EFLKTLNLSD IPTQDSAKVD PPEYMLELYN KFATDRTSMP
SANIIRSFKN EDLFSQPVSF NGLRKYPLLF NVSIPHHEEV IMAELRLYTL
VQRDRMIYDG VDRKITIFEV LESKGDNEGE RNMLVLVSGE IYGTNSEWET
FDVTDAIRRW QKSGSSTHQL EVHIESKHDE AEDASSGRLE IDTSAQNKHN
PLLIVFSDDQ SSDKERKEEL NEMISHEQLP ELDNLGLDSF SSGPGEEALL
QMRSNIIYDS TARIRR (SEQ ID NO: 3)

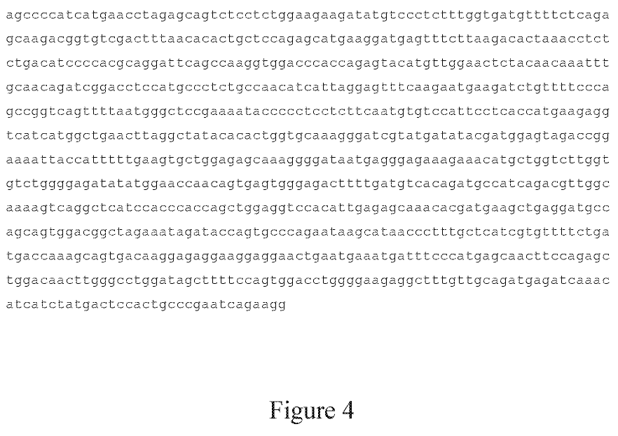

FIG. 4 shows a nucleic acid sequence encoding the BMP 10 propeptide corresponding to SEQ ID NO: 3 (this nucleic acid is designated SEQ ID NO: 4).

The BMP10 propeptide is conserved among vertebrates. Therefore one could generate an alignment of BMP 10 propeptide sequences from different vertebrates using techniques well known in the art and as described herein, and use these alignments to predict key amino acid positions within the propeptides domain that are important for mature BMP10-binding activities as well as to predict amino acid positions that are likely to be tolerant to substitution without significantly altering mature BMP10-binding activities. Therefore, an active, human BMP10pro variant polypeptide useful in accordance with the presently disclosed methods may include one or more amino acids at corresponding positions from the sequence of another vertebrate BMP10pro polypeptide, or may include a residue that is similar to that in the human or other vertebrate sequences.

As shown herein, a variant BMP 10pro polypeptide comprising a BMP10pro domain having a deletion of the N-terminal glycine of the propeptide sequence (deletion of the amino acid at position 1 of SEQ ID NO: 3) and a deletion of the C-terminal arginine of the propeptide sequence (deletion of the amino acid at position 296 of SEQ ID NO: 3) retains high affinity for BMP 10 and can be used as a BMP 10 antagonist. Another variant BMP10pro polypeptide was generated comprising a BMP10pro domain having a deletion of the N-terminal glycine of the propeptide sequence (deletion of the amino acid at position 1 of SEQ ID NO: 3) and a deletion of four amino acids at the C-terminus of the propeptide sequence (deletion of amino acids at positions 293-296 of SEQ ID NO: 3). Surprisingly, the variant BMP10pro polypeptide having four amino acids deleted from the C-terminus of the propeptide sequence was a more potent antagonist of BMP10 activity than a BMP10pro polypeptide having a deletion of only the C-terminal arginine. Thus, BMP10pro polypeptide domains that stop at any one of amino acids 292, 293, 294, 295 and 296 with respect to SEQ ID NO: 3 are all expected to be active, but constructs stopping at 292 may be most active. Any of these forms may be desirable to use, depending on the clinical or experimental setting.

BMP10pro polypeptides may additionally include any of various leader sequences at the N-terminus. Such a sequence would allow the peptides to be expressed and targeted to the secretion pathway in a eukaryotic system. See, e.g., Ernst et al., U.S. Pat. No. 5,082,783 (1992). Alternatively, a native BMP 10 signal sequence may be used to effect extrusion from the cell. Possible leader sequences include honeybee mellitin, TPA, and native leaders, which are disclosed herein. Examples of BMP10pro-Fc fusion proteins incorporating a TPA leader sequence include SEQ ID NOs: 56 and 59. Processing of signal peptides may vary depending on the leader sequence chosen, the cell type used and culture conditions, among other variables, and therefore actual N-terminal start sites for mature BMP10pro polypeptides may shift by 1, 2, 3, 4 or 5 amino acids at the N-terminal direction. Therefore, at the N-terminus of the BMP10pro, it is expected that a protein beginning any one of amino acids 1, 2, 3, 4, 5, or 6 with respect to SEQ ID NO: 3 are all expected to be active.

Taken together, a general formula for an active portion (e.g., mature BMP10-binding portion) of BMP10pro comprises amino acids 6-292 of SEQ ID NO: 3. Therefore, BMP10pro polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of BMP10pro beginning at a residue corresponding to any one of amino acids 1-6 (e.g., beginning at any one of amino acids 1, 2, 3, 4, 5, or 6) of SEQ ID NO: 3 and ending at a position corresponding to any one amino acids 292-296 (e.g., ending at any one of amino acids 292, 293, 294, 295, or 296) of SEQ ID NO: 3. For example, in some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 1-296 of SEQ ID NO: 3. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 1-295 of SEQ ID NO: 3. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 1-294 of SEQ ID NO: 3. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 1-293 of SEQ ID NO: 3. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 1-292 of SEQ ID NO: 3. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 2-295 of SEQ ID NO: 3. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 2-292 of SEQ ID NO: 3. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 3-295 of SEQ ID NO: 3. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 3-294 of SEQ ID NO: 3. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 3-292 of SEQ ID NO: 3. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 4-292 of SEQ ID NO: 3. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 5-292 of SEQ ID NO: 3. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 6-292 of SEQ ID NO: 3. Preferably, BMP10pro polypeptides are soluble. It is expected that the BMP10pro polypeptides described above will retain mature BMP10-binding and antagonizing activity. In some embodiments, such BMP10pro polypeptides may further binds to one or more of BMP9, BMP6, BMP3b and BMP5.

The term “BMPRII polypeptide” includes polypeptides comprising any naturally occurring polypeptide of a BMPRII family member as well as any variants thereof (including mutants, fragments, fusions, and peptidomimetic forms) that retain a useful activity. Proteins described herein are the human forms unless otherwise specified. Numbering of amino acids for all BMPRII-related polypeptides described herein is based on the numbering of the human BMPRII precursor protein sequence provided below (SEQ ID NO: 5), unless specifically designated otherwise.

The amino acid sequence of the unprocessed canonical isoform of human BMPRII precursor (NCBI Reference Sequence NP_001195.2) is as follows:

1 MTSSLQRPWR VPWLPWTILL VSTAAASQNQ ERLCAFKDPY QQDLGIGESR
51 ISHENGTILC SKGSTCYGLW EKSKGDINLV KQGCWSHIGD PQECHYEECV
101 VTTTPPSIQN GTYRFCCCST DLCNVNFTEN FPPPDTTPLS PPHSFNRDET
151 IIIALASVSV LAVLIVALCF GYRMLTGDRK QGLHSMNMME AAASEPSLDL
201 DNLKLLELIG RGRYGAVYKG SLDERPVAVK VFSFANRQNF INEKNIYRVP
251 LMEHDNIARF IVGDERVTAD GRMEYLLVME YYPNGSLCKY LSLHTSDWVS
301 SCRLAHSVTR GLAYLHTELP RGDHYKPAIS HRDLNSRNVL VKNDGTCVIS
351 DFGLSMRLTG NRLVRPGEED NAAISEVGTI RYMAPEVLEG AVNLRDCESA
401 LKQVDMYALG LIYWEIFMRC TDLFPGESVP EYQMAFQTEV GNHPTFEDMQ
451 VLVSREKQRP KFPEAWKENS LAVRSLKETI EDCWDQDAEA RLTAQCAEER
501 MAELMMIWER NKSVSPTVNP MSTAMQNERN LSHNRRVPKI GPYPDYSSSS
551 YIEDSIHHTD SIVKNISSEH SMSSTPLTIG EKNRNSINYE RQQAQARIPS
601 PETSVTSLST NTTTTNTTGL TPSTGMTTIS EMPYPDETNL HTTNVAQSIG
651 PTPVCLQLTE EDLETNKLDP KEVDKNLKES SDENLMEHSL KQFSGPDPLS
701 STSSSLLYPL IKLAVEATGQ QDFTQTANGQ ACLIPDVLPT QIYPLPKQQN
751 LPKRPTSLPL NTKNSTKEPR LKFGSKHKSN LKQVETGVAK MNTINAAEPH
801 VVTVTMNGVA GRNHSVNSHA ATTQYANGTV LSGQTTNIVT HRAQEMLQNQ
851 FIGEDTRLNI NSSPDEHEPL LRREQQAGHD EGVLDRLVDR RERPLEGGRT
901 NSNNNNSNPC SEQDVLAQGV PSTAADPGPS KPRRAQRPNS LDLSATNVLD
951 GSSIQIGEST QDGKSGSGEK IKKRVKTPYS LKRWRPSTWV ISTESLDCEV
1001 NNNGSNRAVH SKSSTAVYLA EGGTATTMVS KDIGMNCL
(SEQ ID NO: 5)

The signal peptide is underlined, and the extracellular domain is indicated in bold.

The sequence of a processed extracellular BMPRII polypeptide (SEQ ID NO: 6) is as follows:

1 SQNQERLCAF KDPYQQDLGI GESRISHENG TILCSKGSTC YGLWEKSKGD
51 INLVKQGCWS HIGDPQECHY EECVVTTTPP SIQNGTYRFC CCSTDLCNVN
101 FTENFPPPDT TPLSPPHSFN RDET (SEQ ID NO: 6)

Based on the positioning of cysteine residues in the sequence, a BMPRII polypeptide may comprise an amino acid sequence beginning at amino acid 1, 2, 3, 4, 5, 6, 7 or 8 of SEQ ID NO: 6 and ending at any of amino acids 97-124 of SEQ ID NO: 6. A nucleic acid sequence encoding the canonical human BMPRII precursor protein is shown below (SEQ ID NO: 7), corresponding to nucleotides 1149-4262 of NCBIReference Sequence NM_001204.6. The signal sequence is underlined.

ATGACTTCCTCGCTGCAGCGGCCCTGGCGGGTGCCCTGGCTACCATGGA CCATCCTGCTGGTCAGCACTGCGGCTGCTTCGCAGAATCAAGAACGGCTA
TGTGCGTTTAAAGATCCGTATCAGCAAGACCTTGGGATAGGTGAGAGTAG
AATCTCTCATGAAAATGGGACAATATTATGCTCGAAAGGTAGCACCTGCT
ATGGCCTTTGGGAGAAATCAAAAGGGGACATAAATCTTGTAAAACAAGGA
TGTTGGTCTCACATTGGAGATCCCCAAGAGTGTCACTATGAAGAATGTGT
AGTAACTACCACTCCTCCCTCAATTCAGAATGGAACATACCGTTTCTGCT
GTTGTAGCACAGATTTATGTAATGTCAACTTTACTGAGAATTTTCCACCT
CCTGACACAACACCACTCAGTCCACCTCATTCATTTAACCGAGATGAGAC
AATAATCATTGCTTTGGCATCAGTCTCTGTATTAGCTGTTTTGATAGTTG
CCTTATGCTTTGGATACAGAATGTTGACAGGAGACCGTAAACAAGGTCTT
CACAGTATGAACATGATGGAGGCAGCAGCATCCGAACCCTCTCTTGATCT
AGATAATCTGAAACTGTTGGAGCTGATTGGCCGAGGTCGATATGGAGCAG
TATATAAAGGCTCCTTGGATGAGCGTCCAGTTGCTGTAAAAGTGTTTTCC
TTTGCAAACCGTCAGAATTTTATCAACGAAAAGAACATTTACAGAGTGCC
TTTGATGGAACATGACAACATTGCCCGCTTTATAGTTGGAGATGAGAGAG
TCACTGCAGATGGACGCATGGAATATTTGCTTGTGATGGAGTACTATCCC
AATGGATCTTTATGCAAGTATTTAAGTCTCCACACAAGTGACTGGGTAAG
CTCTTGCCGTCTTGCTCATTCTGTTACTAGAGGACTGGCTTATCTTCACA
CAGAATTACCACGAGGAGATCATTATAAACCTGCAATTTCCCATCGAGAT
TTAAACAGCAGAAATGTCCTAGTGAAAAATGATGGAACCTGTGTTATTAG
TGACTTTGGACTGTCCATGAGGCTGACTGGAAATAGACTGGTGCGCCCAG
GGGAGGAAGATAATGCAGCCATAAGCGAGGTTGGCACTATCAGATATATG
GCACCAGAAGTGCTAGAAGGAGCTGTGAACTTGAGGGACTGTGAATCAGC
TTTGAAACAAGTAGACATGTATGCTCTTGGACTAATCTATTGGGAGATAT
TTATGAGATGTACAGACCTCTTCCCAGGGGAATCCGTACCAGAGTACCAG
ATGGCTTTTCAGACAGAGGTTGGAAACCATCCCACTTTTGAGGATATGCA
GGTTCTCGTGTCTAGGGAAAAACAGAGACCCAAGTTCCCAGAAGCCTGGA
AAGAAAATAGCCTGGCAGTGAGGTCACTCAAGGAGACAATCGAAGACTGT
TGGGACCAGGATGCAGAGGCTCGGCTTACTGCACAGTGTGCTGAGGAAAG
GATGGCTGAACTTATGATGATTTGGGAAAGAAACAAATCTGTGAGCCCAA
CAGTCAATCCAATGTCTACTGCTATGCAGAATGAACGCAACCTGTCACAT
AATAGGCGTGTGCCAAAAATTGGTCCTTATCCAGATTATTCTTCCTCCTC
ATACATTGAAGACTCTATCCATCATACTGACAGCATCGTGAAGAATATTT
CCTCTGAGCATTCTATGTCCAGCACACCTTTGACTATAGGGGAAAAAAAC
CGAAATTCAATTAACTATGAACGACAGCAAGCACAAGCTCGAATCCCCAG
CCCTGAAACAAGTGTCACCAGCCTCTCCACCAACACAACAACCACAAACA
CCACAGGACTCACGCCAAGTACTGGCATGACTACTATATCTGAGATGCCA
TACCCAGATGAAACAAATCTGCATACCACAAATGTTGCACAGTCAATTGG
GCCAACCCCTGTCTGCTTACAGCTGACAGAAGAAGACTTGGAAACCAACA
AGCTAGACCCAAAAGAAGTTGATAAGAACCTCAAGGAAAGCTCTGATGAG
AATCTCATGGAGCACTCTCTTAAACAGTTCAGTGGCCCAGACCCACTGAG
CAGTACTAGTTCTAGCTTGCTTTACCCACTCATAAAACTTGCAGTAGAAG
CAACTGGACAGCAGGACTTCACACAGACTGCAAATGGCCAAGCATGTTTG
ATTCCTGATGTTCTGCCTACTCAGATCTATCCTCTCCCCAAGCAGCAGAA
CCTTCCCAAGAGACCTACTAGTTTGCCTTTGAACACCAAAAATTCAACAA
AAGAGCCCCGGCTAAAATTTGGCAGCAAGCACAAATCAAACTTGAAACAA
GTCGAAACTGGAGTTGCCAAGATGAATACAATCAATGCAGCAGAACCTCA
TGTGGTGACAGTCACCATGAATGGTGTGGCAGGTAGAAACCACAGTGTTA
ACTCCCATGCTGCCACAACCCAATATGCCAATGGGACAGTACTATCTGGC
CAAACAACCAACATAGTGACACATAGGGCCCAAGAAATGTTGCAGAATCA
GTTTATTGGTGAGGACACCCGGCTGAATATTAATTCCAGTCCTGATGAGC
ATGAGCCTTTACTGAGACGAGAGCAACAAGCTGGCCATGATGAAGGTGTT
CTGGATCGTCTTGTGGACAGGAGGGAACGGCCACTAGAAGGTGGCCGAAC
TAATTCCAATAACAACAACAGCAATCCATGTTCAGAACAAGATGTTCTTG
CACAGGGTGTTCCAAGCACAGCAGCAGATCCTGGGCCATCAAAGCCCAGA
AGAGCACAGAGGCCTAATTCTCTGGATCTTTCAGCCACAAATGTCCTGGA
TGGCAGCAGTATACAGATAGGTGAGTCAACACAAGATGGCAAATCAGGAT
CAGGTGAAAAGATCAAGAAACGTGTGAAAACTCCCTATTCTCTTAAGCGG
TGGCGCCCCTCCACCTGGGTCATCTCCACTGAATCGCTGGACTGTGAAGT
CAACAATAATGGCAGTAACAGGGCAGTTCATTCCAAATCCAGCACTGCTG
TTTACCTTGCAGAAGGAGGCACTGCTACAACCATGGTGTCTAAAGATATA
GGAATGAACTGTCTG (SEQ ID NO:7)

A nucleic acid sequence encoding processed extracellular BMPRII polypeptide (SEQ ID NO: 8) is as follows:

1 TCGCAGAATC AAGAACGGCT ATGTGCGTTT AAAGATCCGT ATCAGCAAGA
51 CCTTGGGATA GGTGAGAGTA GAATCTCTCA TGAAAATGGG ACAATATTAT
101 GCTCGAAAGG TAGCACCTGC TATGGCCTTT GGGAGAAATC AAAAGGGGAC
151 ATAAATCTTG TAAAACAAGG ATGTTGGTCT CACATTGGAG ATCCCCAAGA
201 GTGTCACTAT GAAGAATGTG TAGTAACTAC CACTCCTCCC TCAATTCAGA
251 ATGGAACATA CCGTTTCTGC TGTTGTAGCA CAGATTTATG TAATGTCAAC
301 TTTACTGAGA ATTTTCCACC TCCTGACACA ACACCACTCA GTCCACCTCA
351 TTCATTTAAC CGAGATGAGA CA (SEQ ID NO: 8)

A shorter isoform of human BMPRII precursor (isoform A) has been reported, which contains the same extracellular domain sequence as the canonical BMPRII precursor above. The amino acid sequence of human BMPRII precursor isoform A (NCBI Accession Number AAA86519.1) is as follows:

1 MTSSLQRPWR VPWLPWTILL VSTAAASQNQ ERLCAFKDPY QQDLGIGESR
51 ISHENGTILC SKGSTCYGLW EKSKGDINLV KQGCWSHIGD PQECHYEECV
101 VTTTPPSIQN GTYRFCCCST DLCNVNFTEN FPPPDTTPLS PPHSFNRDET
151 IIIALASVSV LAVLIVALCF GYRMLTGDRK QGLHSMNMME AAASEPSLDL
201 DNLKLLELIG RGRYGAVYKG SLDERPVAVK VFSFANRQNF INEKNIYRVP
251 LMEHDNIARF IVGDERVTAD GRMEYLLVME YYPNGSLCKY LSLHTSDWVS
301 SCRLAHSVTR GLAYLHTELP RGDHYKPAIS HRDLNSRNVL VKNDGTCVIS
351 DFGLSMRLTG NRLVRPGEED NAAISEVGTI RYMAPEVLEG AVNLRDCESA
401 LKQVDMYALG LIYWEIFMRC TDLFPGESVP EYQMAFQTEV GNHPTFEDMQ
451 VLVSREKQRP KFPEAWKENS LAVRSLKETI EDCWDQDAEA RLTAQCAEER
501 MAELMMIWER NKSVSPTVNP MSTAMQNERR (SEQ ID NO: 9)

The signal peptide is underlined, and the extracellular domain is indicated in bold.

A nucleic acid sequence encoding isoform A of the human BMPRII precursor protein is shown below (SEQ ID NO: 10), corresponding to nucleotides 163-1752 of NCBI accession number U25110.1. The signal sequence is underlined.

ATGACTTCCTCGCTGCAGCGGCCCTGGCGGGTGCCCTGGCTACCATGGAC CATCCTGCTGGTCAGCACTGCGGCTGCTTCGCAGAATCAAGAACGGCTAT
GTGCGTTTAAAGATCCGTATCAGCAAGACCTTGGGATAGGTGAGAGTAGA
ATCTCTCATGAAAATGGGACAATATTATGCTCGAAAGGTAGCACCTGCTA
TGGCCTTTGGGAGAAATCAAAAGGGGACATAAATCTTGTAAAACAAGGAT
GTTGGTCTCACATTGGAGATCCCCAAGAGTGTCACTATGAAGAATGTGTA
GTAACTACCACTCCTCCCTCAATTCAGAATGGAACATACCGTTTCTGCTG
TTGTAGCACAGATTTATGTAATGTCAACTTTACTGAGAATTTTCCACCTC
CTGACACAACACCACTCAGTCCACCTCATTCATTTAACCGAGATGAGACA
ATAATCATTGCTTTGGCATCAGTCTCTGTATTAGCTGTTTTGATAGTTGC
CTTATGCTTTGGATACAGAATGTTGACAGGAGACCGTAAACAAGGTCTTC
ACAGTATGAACATGATGGAGGCAGCAGCATCCGAACCCTCTCTTGATCTA
GATAATCTGAAACTGTTGGAGCTGATTGGCCGAGGTCGATATGGAGCAGT
ATATAAAGGCTCCTTGGATGAGCGTCCAGTTGCTGTAAAAGTGTTTTCCT
TTGCAAACCGTCAGAATTTTATCAACGAAAAGAACATTTACAGAGTGCCT
TTGATGGAACATGACAACATTGCCCGCTTTATAGTTGGAGATGAGAGAGT
CACTGCAGATGGACGCATGGAATATTTGCTTGTGATGGAGTACTATCCCA
ATGGATCTTTATGCAAGTATTTAAGTCTCCACACAAGTGACTGGGTAAGC
TCTTGCCGTCTTGCTCATTCTGTTACTAGAGGACTGGCTTATCTTCACAC
AGAATTACCACGAGGAGATCATTATAAACCTGCAATTTCCCATCGAGATT
TAAACAGCAGAAATGTCCTAGTGAAAAATGATGGAACCTGTGTTATTAGT
GACTTTGGACTGTCCATGAGGCTGACTGGAAATAGACTGGTGCGCCCAGG
GGAGGAAGATAATGCAGCCATAAGCGAGGTTGGCACTATCAGATATATGG
CACCAGAAGTGCTAGAAGGAGCTGTGAACTTGAGGGACTGTGAATCAGCT
TTGAAACAAGTAGACATGTATGCTCTTGGACTAATCTATTGGGAGATATT
TATGAGATGTACAGACCTCTTCCCAGGGGAATCCGTACCAGAGTACCAGA
TGGCTTTTCAGACAGAGGTTGGAAACCATCCCACTTTTGAGGATATGCAG
GTTCTCGTGTCTAGGGAAAAACAGAGACCCAAGTTCCCAGAAGCCTGGAA
AGAAAATAGCCTGGCAGTGAGGTCACTCAAGGAGACAATCGAAGACTGTT
GGGACCAGGATGCAGAGGCTCGGCTTACTGCACAGTGTGCTGAGGAAAGG
ATGGCTGAACTTATGATGATTTGGGAAAGAAACAAATCTGTGAGCCCAAC
AGTCAATCCAATGTCTACTGCTATGCAGAATGAACGTAGG (SEQ ID N
O: 10)

A defining structural motif known as a three-finger toxin fold is important for ligand binding by TGFbeta superfamily type I and type IIreceptors and is formed by 10, 12, or 14 conserved cysteine residues located at varying positions within the extracellular domain of each monomeric receptor. See, e.g., Greenwald et al. (1999) Nat Struct Biol 6:18-22; Galat (2011) Cell Mol Life Sci 68:3437-3451; Hinck (2012) FEBS Lett 586:1860-1870. The core ligand-binding domain of a BMPRII receptor, as demarcated by the outermost of these conserved cysteines, comprises positions 34-123 of SEQ ID NO: 5. It is expected that a BMPRII polypeptide beginning at amino acid 34 (the initial cysteine of the ECD), or before, of SEQ ID NO: 5 and ending at amino acid 123 (the last cysteine of the ECD), or after, of SEQ ID NO: 5 will retain ligand binding activity. Examples of ligand binding BMPRII polypeptides therefore include, for example, polypeptides comprising an amino acid sequence that begins at any one of amino acids 27-34 (27, 28, 29, 30, 31, 32, 33, or 34) of SEQ ID NO: 5 and ends at any one of amino acids 123-150 (123, 124, 125, 126, 127, 128, 129, 130, 131, 132, 133, 134, 135, 136, 137, 138, 139, 140, 141, 142, 143, 144, 145, 146, 147, 148, 149, or 150) of SEQ ID NO: 5. In some embodiments, a BMPRII polypeptide of the disclosure comprises an amino acid sequence that is at least 70%, 75%, 80%, 85%, 90%, 91%, 92%, 93%, 94%, 95%, 96%, 97%, 98%, 99%, or 100% identical to amino acids 34-123 of SEQ ID NO: 5. In some embodiments, a BMPRII polypeptide of the disclosure comprises an amino acid sequence that is at least 70%, 75%, 80%, 85%, 90%, 91%, 92%, 93%, 94%, 95%, 96%, 97%, 98%, 99%, or 100% identical to amino acids 27-150 of SEQ ID NO: 5. In some embodiments, a BMPRII polypeptide of the disclosure comprises an amino acid sequence that is at least 70%, 75%, 80%, 85%, 90%, 91%, 92%, 93%, 94%, 95%, 96%, 97%, 98%, 99%, or 100% identical to 27-123 of SEQ ID NO: 5. In some embodiments, a BMPRII polypeptide of the disclosure comprises an amino acid sequence that is at least 70%, 75%, 80%, 85%, 90%, 91%, 92%, 93%, 94%, 95%, 96%, 97%, 98%, 99%, or 100% identical to 34-150 of SEQ ID NO: 5. In certain embodiments, a BMPRII polypeptide binds to BMP9, BMP 10, BMP15 and/or activin B, and the BMPRII polypeptide does not show substantial binding to canonical BMP such as BMP2, BMP4, BMP6 and/or BMP7. Binding may be assessed, for example, using purified proteins in solution or in a surface plasmon resonance system, such as a Biacore™ system.

The term “ALK1 polypeptide” includes polypeptides comprising any naturally occurring polypeptide of an ALK1 family member as well as any variants thereof (including mutants, fragments, fusions, and peptidomimetic forms) that retain a useful activity.

The human ALK1 precursor protein sequence (NCBI Ref Seq NP_000011.2) is as follows:

1 MTLGSPRKGL LMLLMALVTQ GDPVKPSRGP LVTCTCESPH CKGPTCRGAW
51 CTVVLVREEG RHPQEHRGCG NLHRELCRGR PTEFVNHYCC DSHLCNHNVS
101 LVLEATQPPS EQPGTDGQLA LILGPVLALL ALVALGVLGL WHVRRRQEKQ
151 RGLHSELGES SLILKASEQG DSMLGDLLDS DCTTGSGSGL PFLVQRTVAR
201 QVALVECVGK GRYGEVWRGL WHGESVAVKI FSSRDEQSWF RETEIYNTVL
251 LRHDNILGFI ASDMTSRNSS TQLWLITHYH EHGSLYDFLQ RQTLEPHLAL
301 RLAVSAACGL AHLHVEIFGT QGKPAIAHRD FKSRNVLVKS NLQCCIADLG
351 LAVMHSQGSD YLDIGNNPRV GTKRYMAPEV LDEQIRTDCF ESYKWTDIWA
401 FGLVLWEIAR RTIVNGIVED YRPPFYDVVP NDPSFEDMKK WCVDQQTPT
451 IPNRLAADPV LSGLAQMMRE CWYPNPSARL TALRIKKTLQ KISNSPEKPK
501 VIQ (SEQ ID NO: 11)

The signal peptide is indicated by a single underline and the extracellular domain is indicated in bold font.

A processed extracellular ALK1 polypeptide sequence is as follows:

DPVKPSRGPLVTCTCESPHCKGPTCRGAWCTWLVREEGRHPQEHRGCGNL
HRELCRGRPTEFVNHYCCDSHLCNHNVSLVLEATQPPSEQPGTDGQ (SE
Q ID NO: 12)

A nucleic acid sequence encoding human ALK1 precursor protein is shown below (SEQ ID NO: 13), corresponding to nucleotides 284-1792 of Genbank Reference Sequence NM_000020.2. The signal sequence is underlined.

ATGACCTTGGGCTCCCCCAGGAAAGGCCTTCTGATGCTGCTGATGGCCTT GGTGACCCAGGGAGACCCTGTGAAGCCGTCTCGGGGCCCGCTGGTGACCTGCACGTGTGAGAGCCCACATTGCAAGGGGCCTACCTGCCGGGGGGCCTGGTGCACAGTAGTGCTGGTGCGGGAGGAGGGGAGGCACCCCCAGGAACATCGGGGCTGCGGGAACTTGCACAGGGAGCTCTGCAGGGGGCGCCCCACCGAGTTCGTCAACCACTACTGCTGCGACAGCCACCTCTGCAACCACAACGTGTCCCTGGTGCTGGAGGCCACCCAACCTCCTTCGGAGCAGCCGGGAACAGATGGCCAGCTGGCCCTGATCCTGGGCCCCGTGCTGGCCTTGCTGGCCCTGGTGG
CCCTGGGTGTCCTGGGCCTGTGGCATGTCCGACGGAGGCAGGAGAAGCAG
CGTGGCCTGCACAGCGAGCTGGGAGAGTCCAGTCTCATCCTGAAAGCATC
TGAGCAGGGCGACAGCATGTTGGGGGACCTCCTGGACAGTGACTGCACCA
CAGGGAGTGGCTCAGGGCTCCCCTTCCTGGTGCAGAGGACAGTGGCACGG
CAGGTTGCCTTGGTGGAGTGTGTGGGAAAAGGCCGCTATGGCGAAGTGTG
GCGGGGCTTGTGGCACGGTGAGAGTGTGGCCGTCAAGATCTTCTCCTCGA
GGGATGAACAGTCCTGGTTCCGGGAGACTGAGATCTATAACACAGTGTTG
CTCAGACACGACAACATCCTAGGCTTCATCGCCTCAGACATGACCTCCCG
CAACTCGAGCACGCAGCTGTGGCTCATCACGCACTACCACGAGCACGGCT
CCCTCTACGACTTTCTGCAGAGACAGACGCTGGAGCCCCATCTGGCTCTG
AGGCTAGCTGTGTCCGCGGCATGCGGCCTGGCGCACCTGCACGTGGAGAT
CTTCGGTACACAGGGCAAACCAGCCATTGCCCACCGCGACTTCAAGAGCC
GCAATGTGCTGGTCAAGAGCAACCTGCAGTGTTGCATCGCCGACCTGGGC
CTGGCTGTGATGCACTCACAGGGCAGCGATTACCTGGACATCGGCAACAA
CCCGAGAGTGGGCACCAAGCGGTACATGGCACCCGAGGTGCTGGACGAGC
AGATCCGCACGGACTGCTTTGAGTCCTACAAGTGGACTGACATCTGGGCC
TTTGGCCTGGTGCTGTGGGAGATTGCCCGCCGGACCATCGTGAATGGCAT
CGTGGAGGACTATAGACCACCCTTCTATGATGTGGTGCCCAATGACCCCA
GCTTTGAGGACATGAAGAAGGTGGTGTGTGTGGATCAGCAGACCCCCACC
ATCCCTAACCGGCTGGCTGCAGACCCGGTCCTCTCAGGCCTAGCTCAGAT
GATGCGGGAGTGCTGGTACCCAAACCCCTCTGCCCGACTCACCGCGCTGC
GGATCAAGAAGACACTACAAAAAATTAGCAACAGTCCAGAGAAGCCTAAA
GTGATTCAA (SEQ ID NO: 13)

A nucleic acid sequence encoding processed extracelluar ALK1 polypeptide is as follows:

GACCCTGTGAAGCCGTCTCGGGGCCCGCTGGTGACCTGCACGTGTGAGAG
CCCACATTGCAAGGGGCCTACCTGCCGGGGGGCCTGGTGCACAGTAGTGC
TGGTGCGGGAGGAGGGGAGGCACCCCCAGGAACATCGGGGCTGCGGGAAC
TTGCACAGGGAGCTCTGCAGGGGGCGCCCCACCGAGTTCGTCAACCACTA
CTGCTGCGACAGCCACCTCTGCAACCACAACGTGTCCCTGGTGCTGGAGG
CCACCCAACCTCCTTCGGAGCAGCCGGGAACAGATGGCCAG (SEQ ID
NO: 14)

As discussed above, a defining structural motif known as a three-finger toxin fold is important for ligand binding by TGFbeta superfamily type I and type II receptors and is formed by 10, 12, or 14 conserved cysteine residues located at varying positions within the extracellular domain of each monomeric receptor. The core ligand-binding domain of an ALK1 receptor, as demarcated by the outermost of these conserved cysteines, comprises positions 34-95 of SEQ ID NO: 11. It is expected that an ALK1 polypeptide beginning at amino acid 34 (the initial cysteine of the ECD), or before, of SEQ ID NO: 11 and ending at amino acid 95 (the last cysteine of the ECD), or after, of SEQ ID NO: 11 will retain ligand binding activity. Examples of ligand binding ALK1 polypeptides therefore include, for example, polypeptides comprising an amino acid sequence that begins at any one of amino acids 22-34 (22, 23, 24, 25, 26, 27, 28, 29, 30, 31, 32, 33, or 34) of SEQ ID NO: 11 and ends at any one of amino acids 95-118 (95, 96, 97, 98, 99, 100, 101, 102, 103, 104, 105, 106, 107, 108, 109, 110, 111, 112, 113, 114, 115, 116, 117, or 118) of SEQ ID NO: 11. In some embodiments, an ALK1 polypeptide of the disclosure comprises an amino acid sequence that is at least 70%, 75%, 80%, 85%, 90%, 91%, 92%, 93%, 94%, 95%, 96%, 97%, 98%, 99%, or 100% identical to amino acids 34-95 of SEQ ID NO: 11. In some embodiments, an ALK1 polypeptide of the disclosure comprises an amino acid sequence that is at least 70%, 75%, 80%, 85%, 90%, 91%, 92%, 93%, 94%, 95%, 96%, 97%, 98%, 99%, or 100% identical to amino acids 22-118 of SEQ ID NO: 11. In some embodiments, an ALK1 polypeptide of the disclosure comprises an amino acid sequence that is at least 70%, 75%, 80%, 85%, 90%, 91%, 92%, 93%, 94%, 95%, 96%, 97%, 98%, 99%, or 100% identical to 22-95 of SEQ ID NO: 11. In some embodiments, an ALK1 polypeptide of the disclosure comprises an amino acid sequence that is at least 70%, 75%, 80%, 85%, 90%, 91%, 92%, 93%, 94%, 95%, 96%, 97%, 98%, 99%, or 100% identical to 34-95 of SEQ ID NO: 11. In certain embodiments, an ALK1 polypeptide binds to BMP9 and BMP10. Binding may be assessed, for example, using purified proteins in solution or in a surface plasmon resonance system, such as a Biacore™ system.

The term “endoglin polypeptide” includes polypeptides comprising any naturally occurring polypeptide of an endoglin family member as well as any variants thereof (including mutants, fragments, fusions, and peptidomimetic forms) that retain a useful activity.

The human endoglin isoform 1 precursor protein sequence (GenBank NM_001114753) is as follows:

1 MDRGTLPLAV ALLLASCSLS PTSLAETVHC DLQPVGPERG EVTYTTSQVS KGCVAQAPNA
61 ILEVHVLFLE FPTGPSQLEL TLQASKQNGT WPREVLLVLS VNSSVFLHLQ ALGIPLHLAY
121 NSSLVTFQEP PGVNTTELPS FPKTQILEWA AERGPITSAA ELNDPQSILL RLGQAQGSLS
181 FCMLEASQDM GRTLEWRPRT PALVRGCHLE GVAGHKEAHI LRVLPGHSAG PRTVTVKVEL
241 SCAPGDLDAV LILQGPPYVS WLIDANHNMQ IWTTGEYSFK IFPEKNIRGF KLPDTPQGLL
301 GEARMLNASI VASFVELPLA SIVSLHASSC GGRLQTSPAP IQTTPPKDTC SPELLMSLIQ
361 TKCADDAMTL VLKKELVAHL KCTITGLTFW DPSCEAEDRG DKFVLRSAYS SCGMQVSASM
421 ISNEAVVNIL SSSSPQRKKV HCLNMDSLSF QLGLYLSPHF LQASNTIEPG QQSFVQVRVS
481 PSVSEFLLQL DSCHLDLGPE GGTVELIQGR AAKGNCVSLL SPSPEGDPRF SFLLHFYTVP
541 IPKTGTLSCT VALRPKTGSQ DQEVHRTVFM RLNIISPDLS GCTSKGLVLP AVLGITFGAF
601 LIGALLTAAL WYIYSHTRSP SKREPWAVA APASSESSST NHSIGSTQST PCSTSSMA
(SEQ ID NO: 15)

The leader sequence and predicted transmembrane domain are each indicated by a single underline.

A nucleic acid sequence encoding human endoglin isoform 1 precursor protein is shown below (SEQ ID NO: 16; Genbank Reference Sequence NM_001114753). The leader sequence and predicted transmembrane domain are each indicated by a single underline.

1ATGGACCGCG GCACGCTCCC TCTGGCTGTT GCCCTGCTGC TGGCCAGCTG
51 CAGCCTCAGC CCCACAAGTC TTGCAGAAAC AGTCCATTGT GACCTTCAGC
101 CTGTGGGCCC CGAGAGGGGC GAGGTGACAT ATACCACTAG CCAGGTCTCG
151 AAGGGCTGCG TGGCTCAGGC CCCCAATGCC ATCCTTGAAG TCCATGTCCT
201 CTTCCTGGAG TTCCCAACGG GCCCGTCACA GCTGGAGCTG ACTCTCCAGG
251 CATCCAAGCA AAATGGCACC TGGCCCCGAG AGGTGCTTCT GGTCCTCAGT
301 GTAAACAGCA GTGTCTTCCT GCATCTCCAG GCCCTGGGAA TCCCACTGCA
351 CTTGGCCTAC AATTCCAGCC TGGTCACCTT CCAAGAGCCC CCGGGGGTCA
401 ACACCACAGA GCTGCCATCC TTCCCCAAGA CCCAGATCCT TGAGTGGGCA
451 GCTGAGAGGG GCCCCATCAC CTCTGCTGCT GAGCTGAATG ACCCCCAGAG
501 CATCCTCCTC CGACTGGGCC AAGCCCAGGG GTCACTGTCC TTCTGCATGC
551 TGGAAGCCAG CCAGGACATG GGCCGCACGC TCGAGTGGCG GCCGCGTACT
601 CCAGCCTTGG TCCGGGGCTG CCACTTGGAA GGCGTGGCCG GCCACAAGGA
651 GGCGCACATC CTGAGGGTCC TGCCGGGCCA CTCGGCCGGG CCCCGGACGG
701 TGACGGTGAA GGTGGAACTG AGCTGCGCAC CCGGGGATCT CGATGCCGTC
751 CTCATCCTGC AGGGTCCCCC CTACGTGTCC TGGCTCATCG ACGCCAACCA
801 CAACATGCAG ATCTGGACCA CTGGAGAATA CTCCTTCAAG ATCTTTCCAG
851 AGAAAAACAT TCGTGGCTTC AAGCTCCCAG ACACACCTCA AGGCCTCCTG
901 GGGGAGGCCC GGATGCTCAA TGCCAGCATT GTGGCATCCT TCGTGGAGCT
951 ACCGCTGGCC AGCATTGTCT CACTTCATGC CTCCAGCTGC GGTGGTAGGC
1001 TGCAGACCTC ACCCGCACCG ATCCAGACCA CTCCTCCCAA GGACACTTGT
1051 AGCCCGGAGC TGCTCATGTC CTTGATCCAG ACAAAGTGTG CCGACGACGC
1101 CATGACCCTG GTACTAAAGA AAGAGCTTGT TGCGCATTTG AAGTGCACCA
1151 TCACGGGCCT GACCTTCTGG GACCCCAGCT GTGAGGCAGA GGACAGGGGT
1201 GACAAGTTTG TCTTGCGCAG TGCTTACTCC AGCTGTGGCA TGCAGGTGTC
1251 AGCAAGTATG ATCAGCAATG AGGCGGTGGT CAATATCCTG TCGAGCTCAT
1301 CACCACAGCG GAAAAAGGTG CACTGCCTCA ACATGGACAG CCTCTCTTTC
1351 CAGCTGGGCC TCTACCTCAG CCCACACTTC CTCCAGGCCT CCAACACCAT
1401 CGAGCCGGGG CAGCAGAGCT TTGTGCAGGT CAGAGTGTCC CCATCCGTCT
1451 CCGAGTTCCT GCTCCAGTTA GACAGCTGCC ACCTGGACTT GGGGCCTGAG
1501 GGAGGCACCG TGGAACTCAT CCAGGGCCGG GCGGCCAAGG GCAACTGTGT
1551 GAGCCTGCTG TCCCCAAGCC CCGAGGGTGA CCCGCGCTTC AGCTTCCTCC
1601 TCCACTTCTA CACAGTACCC ATACCCAAAA CCGGCACCCT CAGCTGCACG
1651 GTAGCCCTGC GTCCCAAGAC CGGGTCTCAA GACCAGGAAG TCCATAGGAC
1701 TGTCTTCATG CGCTTGAACA TCATCAGCCC TGACCTGTCT GGTTGCACAA
1751 GCAAAGGCCT CGTCCTGCCC GCCGTGCTGG GCATCACCTT TGGTGCCTTC
1801 CTCATCGGGG CCCTGCTCAC TGCTGCACTC TGGTACATCT ACTCGCACAC
1851 GCGTTCCCCC AGCAAGCGGG AGCCCGTGGT GGCGGTGGCT GCCCCGGCCT
1901 CCTCGGAGAG CAGCAGCACC AACCACAGCA TCGGGAGCAC CCAGAGCACC
1951 CCCTGCTCCA CCAGCAGCAT GGCA (SEQ ID NO: 16)

The human endoglin isoform 2 precursor protein sequence (GenBank NM_001114753) is as follows:

1 MDRGTLPLAV ALLLASCSLS PTSLAETVHC DLQPVGPERG EVTYTTSQVS KGCVAQAPNA
61 ILEVHVLFLE FPTGPSQLEL TLQASKQNGT WPREVLLVLS VNSSVFLHLQ ALGIPLHLAY
121 NSSLVTFQEP PGVNTTELPS FPKTQILEWA AERGPITSAA ELNDPQSILL RLGQAQGSLS
181 FCMLEASQDM GRTLEWRPRT PALVRGCHLE GVAGHKEAHI LRVLPGHSAG PRTVTVKVEL
241 SCAPGDLDAV LILQGPPYVS WLIDANHNMQ IWTTGEYSFK IFPEKNIRGF KLPDTPQGLL
301 GEARMLNASI VASFVELPLA SIVSLHASSC GGRLQTSPAP IQTTPPKDTC SPELLMSLIQ
361 TKCADDAMTL VLKKELVAHL KCTITGLTFW DPSCEAEDRG DKFVLRSAYS SCGMQVSASM
421 ISNEAVVNIL SSSSPQRKKV HCLNMDSLSF QLGLYLSPHF LQASNTIEPG QQSFVQVRVS
481 PSVSEFLLQL DSCHLDLGPE GGTVELIQGR AAKGNCVSLL SPSPEGDPRF SFLLHFYTVP
541 IPKTGTLSCT VALRPKTGSQ DQEVHRTVFM RLNIISPDLS GCTSKGLVLP AVLGITFGAF
601 LIGALLTAAL WYIYSHTREY PRPPQ (SEQ ID NO: 17)

The leader sequence and predicted transmembrane domain are each indicated by a single underline.

A nucleic acid sequence encoding human ALK1 isoform 2 precursor protein is shown below (SEQ ID NO: 18; Genbank Reference Sequence NM_001114753). The leader sequence and predicted transmembrane domain are each indicated by a single underline.

ATGGACCGCGGCACGCTCCCTCTGGCTGTTGCCCTGCTGCTGGCCAGCTG CAGCCTCAGCCCCACAAGTCTTGCAGAAACAGTCCATTGTGACCTTCAGC
CTGTGGGCCCCGAGAGGGGCGAGGTGACATATACCACTAGCCAGGTCTCG
AAGGGCTGCGTGGCTCAGGCCCCCAATGCCATCCTTGAAGTCCATGTCCT
CTTCCTGGAGTTCCCAACGGGCCCGTCACAGCTGGAGCTGACTCTCCAGG
CATCCAAGCAAAATGGCACCTGGCCCCGAGAGGTGCTTCTGGTCCTCAGT
GTAAACAGCAGTGTCTTCCTGCATCTCCAGGCCCTGGGAATCCCACTGCA
CTTGGCCTACAATTCCAGCCTGGTCACCTTCCAAGAGCCCCCGGGGGTCA
ACACCACAGAGCTGCCATCCTTCCCCAAGACCCAGATCCTTGAGTGGGCA
GCTGAGAGGGGCCCCATCACCTCTGCTGCTGAGCTGAATGACCCCCAGAG
CATCCTCCTCCGACTGGGCCAAGCCCAGGGGTCACTGTCCTTCTGCATGC
TGGAAGCCAGCCAGGACATGGGCCGCACGCTCGAGTGGCGGCCGCGTACT
CCAGCCTTGGTCCGGGGCTGCCACTTGGAAGGCGTGGCCGGCCACAAGGA
GGCGCACATCCTGAGGGTCCTGCCGGGCCACTCGGCCGGGCCCCGGACGG
TGACGGTGAAGGTGGAACTGAGCTGCGCACCCGGGGATCTCGATGCCGTC
CTCATCCTGCAGGGTCCCCCCTACGTGTCCTGGCTCATCGACGCCAACCA
CAACATGCAGATCTGGACCACTGGAGAATACTCCTTCAAGATCTTTCCAG
AGAAAAACATTCGTGGCTTCAAGCTCCCAGACACACCTCAAGGCCTCCTG
GGGGAGGCCCGGATGCTCAATGCCAGCATTGTGGCATCCTTCGTGGAGCT
ACCGCTGGCCAGCATTGTCTCACTTCATGCCTCCAGCTGCGGTGGTAGGC
TGCAGACCTCACCCGCACCGATCCAGACCACTCCTCCCAAGGACACTTGT
AGCCCGGAGCTGCTCATGTCCTTGATCCAGACAAAGTGTGCCGACGACGC
CATGACCCTGGTACTAAAGAAAGAGCTTGTTGCGCATTTGAAGTGCACCA
TCACGGGCCTGACCTTCTGGGACCCCAGCTGTGAGGCAGAGGACAGGGGT
GACAAGTTTGTCTTGCGCAGTGCTTACTCCAGCTGTGGCATGCAGGTGTC
AGCAAGTATGATCAGCAATGAGGCGGTGGTCAATATCCTGTCGAGCTCAT
CACCACAGCGGAAAAAGGTGCACTGCCTCAACATGGACAGCCTCTCTTTC
CAGCTGGGCCTCTACCTCAGCCCACACTTCCTCCAGGCCTCCAACACCAT
CGAGCCGGGGCAGCAGAGCTTTGTGCAGGTCAGAGTGTCCCCATCCGTCT
CCGAGTTCCTGCTCCAGTTAGACAGCTGCCACCTGGACTTGGGGCCTGAG
GGAGGCACCGTGGAACTCATCCAGGGCCGGGCGGCCAAGGGCAACTGTGT
GAGCCTGCTGTCCCCAAGCCCCGAGGGTGACCCGCGCTTCAGCTTCCTCC
TCCACTTCTACACAGTACCCATACCCAAAACCGGCACCCTCAGCTGCACG
GTAGCCCTGCGTCCCAAGACCGGGTCTCAAGACCAGGAAGTCCATAGGAC
TGTCTTCATGCGCTTGAACATCATCAGCCCTGACCTGTCTGGTTGCACAA
GCAAAGGCCTCGTCCTGCCCGCCGTGCTGGGCATCACCTTTGGTGCCTTC
CTCATCGGGGCCCTGCTCACTGCTGCACTCTGGTACATCTACTCGCACAC
GCGTGAGTACCCCAGGCCCCCACAG (SEQ ID NO: 18)

Applicant has previously demonstrated that Fc fusion proteins comprising shorter C-terminally truncated variants of ENG polypeptides display no appreciable binding to TGF-β1 and TGF-β3 but instead display higher affinity binding to BMP9, with a markedly slower dissociation rate, compared to either ENG(26-437)-Fc or an Fc fusion protein comprising the full-length ENG ECD (see, e.g., US 2015/0307588, the teachings of which are incorporated herein by reference in its entirety). Specifically, C-terminally truncated variants ending at amino acids 378, 359, and 346 of SEQ ID NO: 15 were all found to bind BMP9 with substantially higher affinity (and to bind BMP 10 with undiminished affinity) compared to ENG(26-437) or ENG(26-586). However, binding to BMP9 and BMP10 was completely disrupted by more extensive C-terminal truncations to amino acids 332, 329, or 257. Thus, ENG polypeptides that terminate between amino acid 333 and amino acid 378 are all expected to be active, but constructs ending at, or between, amino acids 346 and 359 may be most active. Forms ending at, or between, amino acids 360 and 378 are predicted to trend toward the intermediate ligand binding affinity shown by ENG(26-378). Improvements in other key parameters are expected with certain constructs ending at, or between, amino acids 333 and 378 based on improvements in protein expression and elimination half-life observed with ENG(26-346)-Fc compared to fusion proteins comprising full-length ENG ECD (see, e.g., US 2015/0307588). Any of these truncated variant forms may be desirable to use, depending on the clinical or experimental setting.

At the N-terminus, it is expected that an ENG polypeptide beginning at amino acid 26 (the initial glutamate), or before, of SEQ ID NO: 15 will retain ligand binding activity. As described herein and in US 2015/0307588, an N-terminal truncation to amino acid 61 of SEQ ID NO: 15 abolishes ligand binding, as do more extensive N-terminal truncations. However, as also disclosed herein, consensus modeling of ENG primary sequences indicates that ordered secondary structure within the region defined by amino acids 26-60 of SEQ ID NO: 15 is limited to a four-residue beta strand predicted with high confidence at positions 42-45 of SEQ ID NO: 15 and a two-residue beta strand predicted with very low confidence at positions 28-29 of SEQ ID NO: 15. Thus, an active ENG polypeptide will begin at (or before) amino acid 26, preferentially, or at any of amino acids 27-42 of SEQ ID NO: 15.

Taken together, an active portion of an ENG polypeptide may comprise an amino acid sequence beginning at any one of amino acids 27-42 (e.g., 27, 28, 29, 30, 31, 32, 33, 34, 35, 36, 37, 38, 39, 40, 41, or 42) of SEQ ID NO: 15 and ending at any one of amino acids 333-378 (333, 334, 335, 336, 337, 338, 339, 340, 341, 342, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 277, of 378) of SEQ ID NO: 15, as well as sequence having at least 70%, 75%, 80%, 85%, 90%, 91%, 92%, 93%, 94%, 95%, 96%, 97%, 98%, or 99% identity to the corresponding portion of SEQ ID NO: 15. For example, active ENG polypeptides may comprise amino acid sequences 26-333, 26-334, 26-335, 26-336, 26-337, 26-338, 26-339, 26-340, 26-341, 26-342, 26-343, 26-344, 26-345, or 26-346 of SEQ ID NO: 15, as well as variants of these sequences starting at any of amino acids 27-42 of SEQ ID NO: 15. Exemplary ENG polypeptides comprise amino acid sequences 26-346, 26-359, and 26-378 of SEQ ID NO: 15. Variants within these ranges are also contemplated, particularly those having at least 70%, 75%, 80%, 85%, 90%, 91%, 92%, 93%, 94%, 95%, 96%, 97%, 98%, or 99% identity to the corresponding portion of SEQ ID NO: 15. An ENG polypeptide may not include the sequence consisting of amino acids 379-430 of SEQ ID NO: 15. In certain embodiments, an ENG polypeptide binds to BMP-9 and BMP-10, and the ENG polypeptide does not show substantial binding to TGF-β1 or TGF-β3. Binding may be assessed using purified proteins in solution or in a surface plasmon resonance system, such as a Biacore™ system.

ENG polypeptides may additionally include any of various leader sequences at the N-terminus. Such a sequence would allow the peptides to be expressed and targeted to the secretion pathway in a eukaryotic system. See, e.g., Ernst et al., U.S. Pat. No. 5,082,783 (1992). Alternatively, a native ENG signal sequence may be used to effect extrusion from the cell. Possible leader sequences include honeybee mellitin, TPA, and native leaders. Processing of signal peptides may vary depending on the leader sequence chosen, the cell type used and culture conditions, among other variables, and therefore actual N-terminal start sites for mature ENG polypeptides may shift by 1, 2, 3, 4 or 5 amino acids in either the N-terminal or C-terminal direction. Examples of mature ENG-Fc fusion proteins include SEQ ID NOs: 19-22, as shown below with the ENG polypeptide portion underlined.

Human ENG(26-378)-hFc (truncated Fc)

ETVHCD LQPVGPERDE VTYTTSQVSK GCVAQAPNAI LEVHVLFLEF
PTGPSQLELT LQASKQNGTW PREVLLVLSV NSSVFLHLQA LGIPLHLAYN
SSLVTFQEPP GVNTTELPSF PKTQILEWAA ERGPITSAAE LNDPQSILLR
LGQAQGSLSF CMLEASQDMG RTLEWRPRTP ALVRGCHLEG VAGHKEAHIL
RVLPGHSAGP RTVTVKVELS CAPGDLDAVL ILQGPPYVSW LIDANHNMQI
WTTGEYSFKI FPEKNIRGFK LPDTPQGLLG EARMLNASIV ASFVELPLAS
IVSLHASSCG GRLQTSPAPI QTTPPKDTCS PELLMSLIQT KCADDAMTLV
LKKELVATGG GTHTCPPCPA PELLGGPSVF LFPPKPKDTL MISRTPEVTC
VVVDVSHEDP EVKFNWYVDG VEVHNAKTKP REEQYNSTYR VVSVLTVLHQ
DWLNGKEYKC KVSNKALPAP IEKTISKAKG QPREPQVYTL PPSREEMTKN
QVSLTCLVKG FYPSDIAVEW ESNGQPENNY KTTPPVLDSD GSFFLYSKLT
VDKSRWQQGN VFSCSVMHEA LHNHYTQKSL SLSPGK (SEQ ID NO: 19)

Human ENG(26-359)-hFc

ETVHCD LQPVGPERDE VTYTTSQVSK GCVAQAPNAI LEVHVLFLEF
PTGPSQLELT LQASKQNGTW PREVLLVLSV NSSVFLHLQA LGIPLHLAYN
SSLVTFQEPP GVNTTELPSF PKTQILEWAA ERGPITSAAE LNDPQSILLR
LGQAQGSLSF CMLEASQDMG RTLEWRPRTP ALVRGCHLEG VAGHKEAHIL
RVLPGHSAGP RTVTVKVELS CAPGDLDAVL ILQGPPYVSW LIDANHNMQI
WTTGEYSFKI FPEKNIRGFK LPDTPQGLLG EARMLNASIV ASFVELPLAS
IVSLHASSCG GRLQTSPAPI QTTPPKDTCS PELLMSLITG GGPKSCDKTH
TCPPCPAPEL LGGPSVFLFP PKPKDTLMIS RTPEVTCVVV DVSHEDPEVK
FNWYVDGVEV HNAKTKPREE QYNSTYRWS VLTVLHQDWL NGKEYKCKVS
NKALPAPIEK TISKAKGQPR EPQVYTLPPS REEMTKNQVS LTCLVKGFYP
SDIAVEWESN GQPENNYKTT PPVLDSDGSF FLYSKLTVDK SRWQQGNVFS
CSVMHEALHN HYTQKSLSLS PGK (SEQ ID NO: 20)

Human ENG(26-359)-hFc (truncated Fc)

ETVHCD LQPVGPERDE VTYTTSQVSK GCVAQAPNAI LEVHVLFLEF
PTGPSQLELT LQASKQNGTW PREVLLVLSV NSSVFLHLQA LGIPLHLAYN
SSLVTFQEPP GVNTTELPSF PKTQILEWAA ERGPITSAAE LNDPQSILLR
LGQAQGSLSF CMLEASQDMG RTLEWRPRTP ALVRGCHLEG VAGHKEAHIL
RVLPGHSAGP RTVTVKVELS CAPGDLDAVL ILQGPPYVSW LIDANHNMQI
WTTGEYSFKI FPEKNIRGFK LPDTPQGLLG EARMLNASIV ASFVELPLAS
IVSLHASSCG GRLQTSPAPI QTTPPKDTCS PELLMSLITG GGTHTCPPCP
APELLGGPSV FLFPPKPKDT LMISRTPEVT CVVVDVSHED PEVKFNWYVD
GVEVHNAKTK PREEQYNSTY RVVSVLTVLH QDWLNGKEYK CKVSNKALPA
PIEKTISKAK GQPREPQVYT LPPSREEMTK NQVSLTCLVK GFYPSDIAVE
WESNGQPENN YKTTPPVLDS DGSFFLYSKL TVDKSRWQQG NVFSCSVMHE
ALHNHYTQKS LSLSPGK (SEQ ID NO: 21)

Human ENG(26-346)-hFc (truncated Fc)

ETVHCD LQPVGPERDE VTYTTSQVSK GCVAQAPNAI LEVHVLFLEF
PTGPSQLELT LQASKQNGTW PREVLLVLSV NSSVFLHLQA LGIPLHLAYN
SSLVTFQEPP GVNTTELPSF PKTQILEWAA ERGPITSAAE LNDPQSILLR
LGQAQGSLSF CMLEASQDMG RTLEWRPRTP ALVRGCHLEG VAGHKEAHIL
RVLPGHSAGP RTVTVKVELS CAPGDLDAVL ILQGPPYVSW LIDANHNMQI
WTTGEYSFKI FPEKNIRGFK LPDTPQGLLG EARMLNASIV ASFVELPLAS
IVSLHASSCG GRLQTSPAPI QTTPPTGGGT HTCPPCPAPE LLGGPSVFLF
PPKPKDTLMI SRTPEVTCVV VDVSHEDPEV KFNWYVDGVE VHNAKTKPRE
EQYNSTYRVV SVLTVLHQDW LNGKEYKCKV SNKALPAPIE KTISKAKGQP
REPQVYTLPP SREEMTKNQV SLTCLVKGFY PSDIAVEWES NGQPENNYKT
TPPVLDSDGS FFLYSKLTVD KSRWQQGNVF SCSVMHEALH NHYTQKSLSL
SPGK (SEQ ID NO: 22)

In some embodiments, the present disclosure contemplates making functional variants by modifying the structure of a BMP 10 propeptide polypeptide, BMPRII polypeptide, ALK1 polypeptide and/or endoglin polypeptide for such purposes as enhancing therapeutic efficacy or stability (e.g., shelf-life and resistance to proteolytic degradation in vivo). Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, or to bind to one or more TGF-beta ligands including, for example, BMP2, BMP2/7, BMP3b, BMP5, BMP6, BMP9, BMP10, TGF-β1, TGF-β3, activin A, activin B, and nodal.

In certain embodiments, the present disclosure contemplates specific mutations of a BMP10 propeptide polypeptide, BMPRII polypeptide, ALK1 polypeptide and/or endoglin polypeptide so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, BMP 10 propeptide polypeptides, BMPRII polypeptides, ALK1 polypeptides and/or endoglin polypeptides of the present disclosure for use in humans may be expressed in a mammalian cell line that provides proper glycosylation, such as HEK293 or CHO cell lines, although other mammalian expression cell lines are expected to be useful as well.

The disclosure further contemplates a method of generating mutants, particularly sets of combinatorial mutants of BMP 10 propeptide polypeptides, BMPRII polypeptides, ALK1 polypeptides and/or endoglin polypeptides as well as truncation mutants. Pools of combinatorial mutants are especially useful for identifying functionally active (e.g., TGF-beta superfamily ligand binding) BMPRII, ALK1, endoglin and/or BMP 10 propeptide sequences. The purpose of screening such combinatorial libraries may be to generate, for example, polypeptides variants which have altered properties, such as altered pharmacokinetic or altered ligand binding. A variety of screening assays are provided below, and such assays may be used to evaluate variants. For example, BMP 10 propeptide polypeptide, BMPRII polypeptide, ALK1 polypeptide and/or endoglin polypeptide variants may be screened for ability to bind to one or more TGF-beta superfamily ligands (e.g., BMP2, BMP2/7, BMP3b, BMP5, BMP6, BMP9, BMP10, TGF-β1, TGF-β3, activin A, activin B, and nodal), to prevent binding of a TGF-beta superfamily ligand to a TGF-beta superfamily receptor, and/or to interfere with signaling caused by an TGF-beta superfamily ligand.

The activity of BMP 10 propeptide polypeptides, BMPRII polypeptides, ALK1 polypeptides and/or endoglin polypeptides also may be tested in a cell-based assay or in vivo. For example, the effect of a BMP10 propeptide polypeptide, BMPRII polypeptide, ALK1 polypeptide and/or endoglin polypeptide on the expression of genes involved in muscle production in a muscle cell may be assessed. This may, as needed, be performed in the presence of one or more recombinant TGF-beta superfamily ligand proteins (e.g., BMP2, BMP2/7, BMP3b, BMP5, BMP6, BMP9, BMP10, TGF-β1, TGF-β3, activin A, activin B, and nodal), and cells may be transfected so as to produce a BMP 10 propeptide polypeptide, BMPRII polypeptide, ALK1 polypeptide and/or endoglin polypeptide, and optionally, a TGF-beta superfamily ligand. In some embodiments, the effect of a BMP 10 propeptide polypeptide, BMPRII polypeptide, ALK1 polypeptide and/or endoglin polypeptide on the ligand-mediated signaling through endogenous ALK1, ALK2, ALK3, BMPRII, or endoglin receptors can be determined. For instance, cells (e.g., human pulmonary artery endothelial cells or human dermal microvascular endothelial cells) can be transfected so as to produce a BMP10 propeptide polypeptide, BMPRII polypeptide, ALK1 polypeptide and/or endoglin polypeptide, and optionally, a TGF-beta superfamily ligand. In some embodiments, the phosphorylation of Smads 1, 5, and 8 in these cells can then be measured using appropriate anti-phospho-Smad 1, 5, or 8 antibodies. In some embodiments, a SMAD-responsive reporter gene may be used in such cell lines to monitor effects on downstream signaling. In some embodiments, the binding affinity of a BMP10 propeptide polypeptide, BMPRII polypeptide, ALK1 polypeptide and/or endoglin polypeptide to a TGF-beta superfamily ligand can be measured using Surface Plasmon Resonance (SPR). In some embodiments, the expression of BMP-regulated target genes (e.g., ID1, BMPR2, HEY1 and HEY2) can be measured (e.g., mRNA gene expression) following treatment of cells with a TGF-beta superfamily ligand.

Combinatorial-derived variants can be generated which have increased selectivity or generally increased potency relative to a reference BMP 10 propeptide polypeptide, BMPRII polypeptide, ALK1 polypeptide and/or endoglin polypeptide. Such variants, when expressed from recombinant DNA constructs, can be used in gene therapy protocols. Likewise, mutagenesis can give rise to variants which have intracellular half-lives dramatically different than the corresponding unmodified BMP 10 propeptide polypeptide, BMPRII polypeptide, ALK1 polypeptide and/or endoglin polypeptide. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide levels by modulating the half-life of the polypeptide. For instance, a short half-life can give rise to more transient biological effects and, when part of an inducible expression system, can allow tighter control of recombinant polypeptide levels within the cell. In an Fc fusion protein, mutations may be made in the linker (if any) and/or the Fc portion to alter the half-life of the BMP 10 propeptide polypeptide, BMPRII polypeptide, ALK1 polypeptide and/or endoglin polypeptide.

A combinatorial library may be produced by way of a degenerate library of genes encoding a library of polypeptides which each include at least a portion of potential BMPRII, ALK1, endoglin, and/or BMP10 propeptide sequences. For instance, a mixture of synthetic oligonucleotides can be enzymatically ligated into gene sequences such that the degenerate set of potential BMPRII, ALK1, endoglin, and/or BMP 10 propeptide encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art [Narang, SA (1983) Tetrahedron 39:3; Itakura et al. (1981) Recombinant DNA, Proc. 3rd Cleveland Sympos. Macromolecules, ed. AG Walton, Amsterdam: Elsevier pp273-289; Itakura et al. (1984) Annu. Rev. Biochem. 53:323; Itakura et al. (1984) Science 198:1056; and Ike et al. (1983) Nucleic Acid Res. 11:477]. Such techniques have been employed in the directed evolution of other proteins [Scott et al., (1990) Science 249:386-390; Roberts et al. (1992) PNAS USA 89:2429-2433; Devlin et al. (1990) Science 249: 404-406; Cwirla et al., (1990) PNAS USA 87: 6378-6382; as well as U.S. Pat. Nos: 5,223,409, 5,198,346, and 5,096,815].

Alternatively, other forms of mutagenesis can be utilized to generate a combinatorial library. For example, BMP10 propeptide polypeptides, BMPRII polypeptides, ALK1 polypeptides and/or endoglin polypeptides of the disclosure can be generated and isolated from a library by screening using, for example, alanine scanning mutagenesis [Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [Gustin et al. (1993) Virology 193:653-660; and Brown et al. (1992) Mol. Cell Biol. 12:2644-2652; McKnight et al. (1982) Science 232:316], by saturation mutagenesis [Meyers et al., (1986) Science 232:613]; by PCR mutagenesis [Leung et al. (1989) Method Cell Mol Biol 1:11-19]; or by random mutagenesis, including chemical mutagenesis [Miller et al. (1992) A Short Course in Bacterial Genetics, CSHL Press, Cold Spring Harbor, NY; and Greener et al. (1994) Strategies in Mol Biol 7:32-34]. Linker scanning mutagenesis, particularly in a combinatorial setting, is an attractive method for identifying truncated (bioactive) forms of BMP 10 propeptide polypeptides, BMPRII polypeptides, ALK1 polypeptides and/or endoglin polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of BMP 10 propeptide polypeptides, BMPRII polypeptides, ALK1 polypeptides and/or endoglin polypeptides. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include BMP (e.g., BMP 10, BMP9, BMP6, BMP3b and BMP5) binding assays and/or BMP-mediated cell signaling assays.

In certain embodiments, BMP 10 propeptide polypeptides, BMPRII polypeptides, ALK1 polypeptides and/or endoglin polypeptides may further comprise post-translational modifications in addition to any that are naturally present in the BMPRII, ALK1, endoglin, and/or BMP 10 propeptide polypeptide. Such modifications include, but are not limited to, acetylation, carboxylation, glycosylation, phosphorylation, lipidation, and acylation. As a result, BMP10 propeptide polypeptides, BMPRII polypeptides, ALK1 polypeptides, and/or endoglin polypeptides may comprise non-amino acid elements, such as polyethylene glycols, lipids, polysaccharide or monosaccharide, and phosphates. Effects of such non-amino acid elements on the functionality of a BMP 10 propeptide polypeptides, BMPRII polypeptides, ALK1 polypeptides and/or endoglin polypeptides may be tested as described herein for other variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, WI38, NIH-3T3 or HEK293) have specific cellular machinery and characteristic mechanisms for such post-translational activities and may be chosen to ensure the correct modification and processing of the BMPRII, ALK1, endoglin, or BMP 10 propeptide polypeptide.

In certain aspects, BMP 10 propeptide polypeptides, BMPRII polypeptides, ALK1 polypeptides and/or endoglin polypeptides of the disclosure are fusion proteins comprising at least a portion (domain) of a BMPRII, ALK1, endoglin, or BMP 10 propeptide polypeptide and one or more heterologous portions (domains). Well-known examples of such fusion domains include, but are not limited to, polyhistidine, Glu-Glu, glutathione S-transferase (GST), thioredoxin, protein A, protein G, an immunoglobulin heavy-chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt- conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the BMP 10 propeptide polypeptide, BMPRII polypeptide, ALK1 polypeptide and/or endoglin polypeptide. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagglutinin (HA), and c-myc tags. In some cases, the fusion domains have a protease cleavage site, such as for Factor Xa or thrombin, which allows the relevant protease to partially digest the fusion proteins and thereby liberate the recombinant proteins therefrom. The liberated proteins can then be isolated from the fusion domain by subsequent chromatographic separation. Other types of fusion domains that may be selected include multimerizing (e.g., dimerizing, tetramerizing) domains and functional domains (that confer an additional biological function) including, for example constant domains from immunoglobulins (e.g., Fc domains).

In certain aspects, BMP10 propeptide polypeptides, BMPRII polypeptides, ALK1 polypeptides and/or endoglin polypeptides of the present disclosure contain one or more modifications that are capable of “stabilizing” the polypeptides. By “stabilizing” is meant anything that increases the in vitro half-life, serum half-life, regardless of whether this is because of decreased destruction, decreased clearance by the kidney, or other pharmacokinetic effect of the agent. For example, such modifications enhance the shelf-life of the polypeptides, enhance circulatory half-life of the polypeptides, and/or reduce proteolytic degradation of the polypeptides. Such stabilizing modifications include, but are not limited to, fusion proteins (including, for example, fusion proteins comprising a BMP10 propeptide polypeptide, BMPRII polypeptide, ALK1 polypeptide and/or endoglin polypeptide domain and a stabilizer domain), modifications of a glycosylation site (including, for example, addition of a glycosylation site to a polypeptide of the disclosure), and modifications of carbohydrate moiety (including, for example, removal of carbohydrate moieties from a polypeptide of the disclosure). As used herein, the term “stabilizer domain” not only refers to a fusion domain (e.g., an immunoglobulin Fc domain) as in the case of fusion proteins, but also includes nonproteinaceous modifications such as a carbohydrate moiety, or nonproteinaceous moiety, such as polyethylene glycol. In certain preferred embodiments, a BMP10 propeptide polypeptide, BMPRII polypeptide, ALK1 polypeptide and/or endoglin polypeptide is fused with a heterologous domain that stabilizes the polypeptide (a “stabilizer” domain), preferably a heterologous domain that increases stability of the polypeptide in vivo. Fusions with a constant domain of an immunoglobulin (e.g., an Fc domain) are known to confer desirable pharmacokinetic properties on a wide range of proteins. Likewise, fusions to human serum albumin can confer desirable stabilizing properties.

In some embodiments, BMPRII, ALK1, endoglin, and/or BMP10 propeptide polypeptides of the disclosure are Fc fusion proteins. An example of a native amino acid sequence that may be used for the Fc portion of human IgG1 (G1Fc) is shown below (SEQ ID NO: 23). Dotted underline indicates the hinge region, and solid underline indicates positions with naturally occurring variants. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 23. Naturally occurring variants in G1Fc would include E134D and M136L according to the numbering system used in SEQ ID NO: 23 (see Uniprot P01857).

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSREEMTKNQ VSLTCLVKGF
151 YPSDIAVEWE SNGQPENNYK TTPPVLDSDG SFFLYSKLTV DKSRWQQGNV
201 FSCSVMHEAL HNHYTQKSLS LSPGK (SEQ ID NO: 23)

Optionally, the IgG1 Fc domain has one or more mutations at residues such as Asp-265, lysine 322, and Asn-434. In certain cases, the mutant IgG1 Fc domain having one or more of these mutations (e.g., Asp-265 mutation) has reduced ability of binding to the Fcy receptor relative to a wild-type Fc domain. In other cases, the mutant Fc domain having one or more of these mutations (e.g., Asn-434 mutation) has increased ability of binding to the MHC class I-related Fc-receptor (FcRN) relative to a wild-type IgG1 Fc domain.

An example of a native amino acid sequence that may be used for the Fc portion of human IgG2 (G2Fc) is shown below (SEQ ID NO: 24). Dotted underline indicates the hinge region and double underline indicates positions where there are data base conflicts in the sequence (according to UniProt P01859). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 24.

1 VECPPCPAPP VAGPSVFLFP PKPKDTLMIS RTPEVTCVVV DVSHEDPEVQ
51 FNWYVDGVEV HNAKTKPREE QFNSTFRVVS VLTVVHQDWL NGKEYKCKVS
101 NKGLPAPIEK TISKTKGQPR EPQVYTLPPS REEMTKNQVS LTCLVKGFYP
151 SDIAVEWESN GQPENNYKTT PPMLDSDGSF FLYSKLTVDK SRWQQGNVFS
201 CSVMHEALHN HYTQKSLSLS PGK (SEQ ID NO: 24)

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 25) contains a short hinge region consisting of a single 15-residue segment, whereas the second G3Fc sequence (SEQ ID NO: 26) contains a full-length hinge region. In each case, dotted underline indicates the hinge region, and solid underline indicates positions with naturally occurring variants according to UniProt P01859. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NOs: 25 or 26.

1 EPKSCDTPPP CPRCPAPELL GGPSVFLFPP KPKDTLMISR TPEVTCVVVD
51 VSHEDPEVQF KWYVDGVEVH NAKTKPREEQ YNSTFRVVSV LTVLHQDWLN
101 GKEYKCKVSN KALPAPIEKT ISKTKGQPRE PQVYTLPPSR EEMTKNQVSL
151 TCLVKGFYPS DIAVEWESSG QPENNYNTTP PMLDSDGSFF LYSKLTVDKS
201 RWQQGNIFSC SVMHEALHNR FTQKSLSLSP GK (SEQ ID NO: 25)

1 ELKTPLGDTT HTCPRCPEPK SCDTPPPCPR CPEPKSCDTP PPCPRCPEPK
51 SCDTPPPCPR CPAPELLGGP SVFLFPPKPK DTLMISRTPE VTCVVVDVSH
101 EDPEVQFKWY VDGVEVHNAK TKPREEQYNS TFRVVSVLTV LHQDWLNGKE
151 YKCKVSNKAL PAPIEKTISK TKGQPREPQV YTLPPSREEM TKNQVSLTCL
201 VKGFYPSDIA VEWESSGQPE NNYNTTPPML DSDGSFFLYS KLTVDKSRWQ
251 QGNIFSCSVM HEALHNRFTQ KSLSLSPGK (SEQ ID NO: 26)

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 25, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 27). Dotted underline indicates the hinge region. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 27.

1 ESKYGPPCPS CPAPEFLGGP SVFLFPPKPK DTLMISRTPE VTCVVVDVSQ
51 EDPEVQFNWY VDGVEVHNAK TKPREEQFNS TYRVVSVLTV LHQDWLNGKE
101 YKCKVSNKGL PSSIEKTISK AKGQPREPQV YTLPPSQEEM TKNQVSLTCL
151 VKGFYPSDIA VEWESNGQPE NNYKTTPPVL DSDGSFFLYS RLTVDKSRWQ
201 EGNVFSCSVM HEALHNHYTQ KSLSLSLGK (SEQ ID NO: 27)

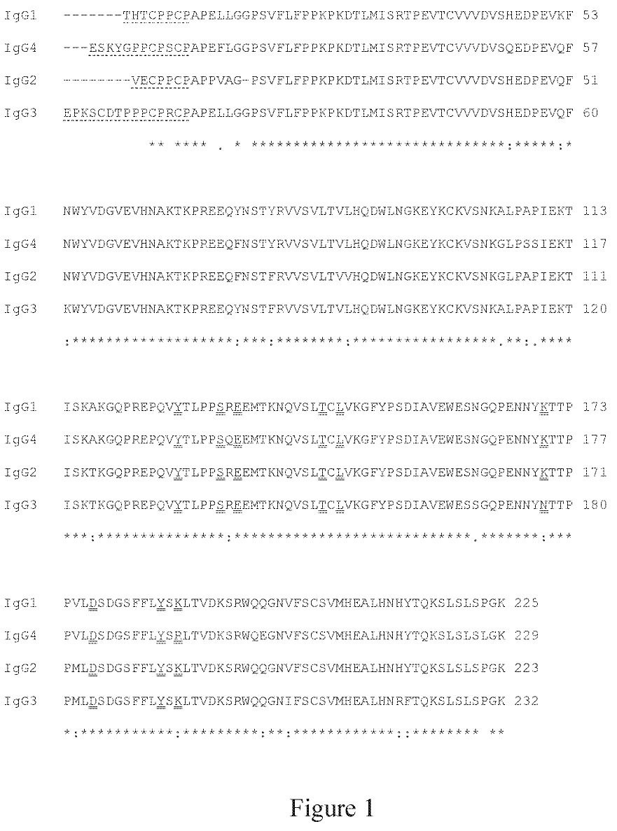

A variety of engineered mutations in the Fc domain are presented herein with respect to the G1Fc sequence (SEQ ID NO: 23), and analogous mutations in G2Fc, G3Fc, and G4Fc can be derived from their alignment with G1Fc in FIG. 1. Due to unequal hinge lengths, analogous Fc positions based on isotype alignment (FIG. 1) possess different amino acid numbers in SEQ ID NOs: 23, 24, 25, 26, and 27. It can also be appreciated that a given amino acid position in an immunoglobulin sequence consisting of hinge, C_{H}2, and C_{H}3 regions (e.g., SEQ ID NOs: 23, 24, 25, 26, and 27) will be identified by a different number than the same position when numbering encompasses the entire IgG1 heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 23), the human IgG1 heavy chain constant domain (Uniprot P01857), and the human IgG1 heavy chain is as follows.

Correspondence of C_{H}3 Positions in Different Numbering Systems

| | | |
|---|---|---|
| G1Fc (Numbering begins at first threonine in hinge region) | IgG1 heavy chain constant domain (Numbering begins at C_{H}1) | IgG1 heavy chain (EU numbering scheme of Kabat et al., 1991 *) |
| Y127 | Y232 | Y349 |
| S132 | S237 | S354 |
| E134 | E239 | E356 |
| T144 | T249 | T366 |
| L146 | L251 | L368 |
| K170 | K275 | K392 |
| D177 | D282 | D399 |
| Y185 | Y290 | Y407 |
| K187 | K292 | K409 |

* Kabat et al. (eds) 1991; pp. 688-696 in Sequences of Proteins of Immunological Interest, 5^{th} ed., Vol. 1, NIH, Bethesda, MD.

The application further provides antibodies and Fc fusion proteins with engineered or variant Fc regions. An antibody of the present disclosure, or combination of antibodies, may bind to, for example, BMP10, BMP9, BMP6, BMP5, and/or BMP3b or one or more BMP-interacting receptors [e.g., BMPRII and endoglin]. Such antibodies and Fc fusion proteins may be useful, for example, in modulating effector functions, such as, antigen-dependent cytotoxicity (ADCC) and complement-dependent cytotoxicity (CDC). Additionally, the modifications may improve the stability of the antibodies and Fc fusion proteins. Amino acid sequence variants of the antibodies and Fc fusion proteins are prepared by introducing appropriate nucleotide changes into the DNA, or by peptide synthesis. Such variants include, for example, deletions from, and/or insertions into and/or substitutions of, residues within the amino acid sequences of the antibodies and Fc fusion proteins disclosed herein. Any combination of deletion, insertion, and substitution is made to arrive at the final construct, provided that the final construct possesses the desired characteristics. The amino acid changes also may alter post-translational processes of the antibodies and Fc fusion proteins, such as changing the number or position of glycosylation sites.

Antibodies and Fc fusion proteins with reduced effector function may be produced by introducing changes in the amino acid sequence, including, but are not limited to, the Ala-Ala mutation described by Bluestone et al. (see WO 94/28027 and WO 98/47531; also see Xu et al. 2000 Cell Immunol 200; 16-26) and the P329G/L234A/L235A (P329G LALA) mutation described by Schlothauer et al. (see Schlothauer T., et al. Protein Eng Des Sel. 2016 Oct;29(10):457-466). Thus in certain embodiments, antibodies and Fc fusion proteins of the disclosure with mutations within the constant region including the Ala-Ala mutation or the P329G LALA mutation may be used to reduce or abolish effector function. According to these embodiments, antibodies and Fc fusion proteins may comprise a mutation to an alanine at position 234 or a mutation to an alanine at position 235, or a combination thereof. In one embodiment, the antibody or Fc fusion protein comprises an IgG4 framework, wherein the Ala-Ala mutation would describe a mutation(s) from phenylalanine to alanine at position 234 and/or a mutation from leucine to alanine at position 235. In some embodiments, antibodies and Fc fusion proteins may further comprise mutation from proline to glycine at position 329. In another embodiment, the antibody or Fc fusion protein comprises an IgG1 framework, wherein the Ala-Ala mutation would describe a mutation(s) from leucine to alanine at position 234 and/or a mutation from leucine to alanine at position 235. In some embodiments, the antibody or Fc fusion protein comprising an IgG1 framework further comprises a mutation from proline to glycine at position 329. The antibody or Fc fusion protein may alternatively or additionally carry other mutations, including the point mutation K322A in the CH2 domain (Hezareh et al. 2001 J Virol. 75: 12161-8).

In some embodiments, the antibody or Fc fusion protein may be modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO99/51642, Duncan & Winter Nature 322: 738-40 (1988); U.S. Pat. No. 5,648,260; U.S. Pat. No. 5,624,821; and WO94/29351.

It is understood that different elements of the fusion proteins (e.g., immunoglobulin Fc fusion proteins) may be arranged in any manner that is consistent with desired functionality. For example, a BMP10 propeptide polypeptide, BMPRII polypeptide, ALK1 polypeptide and/or endoglin polypeptide domain may be placed C-terminal to a heterologous domain, or alternatively, a heterologous domain may be placed C-terminal to a BMP10 propeptide polypeptide, BMPRII polypeptide, ALK1 polypeptide and/or endoglin polypeptide domain. The BMP10 propeptide polypeptide, BMPRII polypeptide, ALK1 polypeptide and/or endoglin polypeptide domain and the heterologous domain need not be adjacent in a fusion protein, and additional domains or amino acid sequences may be included C- or N-terminal to either domain or between the domains.

For example, a BMP10 propeptide (or BMPRII, ALK1, or endoglin) fusion protein may comprise an amino acid sequence as set forth in the formula A-B-C. The B portion corresponds to a BMP 10 propeptide (or BMPRII, ALK1, or endoglin) polypeptide domain. The A and C portions may be independently zero, one, or more than one amino acid, and both the A and C portions when present are heterologous to B. The A and/or C portions may be attached to the B portion via a linker sequence. A linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 28), GGGG (SEQ ID NO: 29), TGGGG(SEQ ID NO: 30), SGGGG (SEQ ID NO: 31), TGGG (SEQ ID NO: 32), SGGG (SEQ ID NO: 33), or GGGGS (SEQ ID NO: 34) singlets, or repeats. In certain embodiments, a BMP10 propeptide (or BMPRII, ALK1, or endoglin) fusion protein comprises an amino acid sequence as set forth in the formula A-B-C, wherein A is a leader (signal) sequence, B consists of a BMP10 propeptide (or BMPRII, ALK1, or endoglin) polypeptide domain, and C is a polypeptide portion that enhances one or more of in vivo stability, in vivo half-life, uptake/administration, tissue localization or distribution, formation of polypeptides, and/or purification. In certain embodiments, a BMP10 propeptide (or BMPRII, ALK1, or endoglin) fusion protein comprises an amino acid sequence as set forth in the formula A-B-C, wherein A is a TPA leader sequence, B consists of a BMP10 propeptide (or BMPRII, ALK1, or endoglin) receptor polypeptide domain, and C is an immunoglobulin Fc domain. Preferred fusion proteins comprise the amino acid sequence set forth in any one of SEQ ID NOs: 19, 20, 21, 22, 44, 46, 49, 50, 52, 54, 56, 58, 59, and 61.

In certain preferred embodiments, a BMP10 propeptide polypeptide, BMPRII polypeptide, ALK1 polypeptide and/or endoglin polypeptide to be used in accordance with the methods described herein are isolated. As used herein, an isolated protein or polypeptide is one which has been separated from a component of its natural environment. In some embodiments, a polypeptide of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of antibody purity are well known in the art [Flatman et al., (2007) J. Chromatogr. B 848:79-87].

In certain embodiments, a BMP10 propeptide polypeptides, BMPRII polypeptides, ALK1 polypeptides and/or endoglin polypeptides of the disclosure can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User’s Guide, W. H. Freeman and Company, New York (1992). In addition, automated peptide synthesizers are commercially available (Advanced ChemTech Model 396; Milligen/Biosearch 9600). Alternatively, the polypeptides the disclosure, including fragments or variants thereof, may be recombinantly produced using various expression systems [E. coli, Chinese Hamster Ovary (CHO) cells, COS cells, baculovirus] as is well known in the art. In a further embodiment, the modified or unmodified polypeptides of the disclosure may be produced by digestion of recombinantly produced full-length a BMP10 propeptide polypeptide, BMPRII polypeptide, ALK1 polypeptide and/or endoglin polypeptide by using, for example, a protease, e.g., trypsin, thermolysin, chymotrypsin, pepsin, or paired basic amino acid converting enzyme (PACE). Computer analysis (using commercially available software, e.g., MacVector, Omega, PCGene, Molecular Simulation, Inc.) can be used to identify proteolytic cleavage sites.

### 3. Linkers

The disclosure provides for BMP antagonists (e.g., BMP10 propeptide polypeptides, ALK1 polypeptides, BMPRII polypeptides, and endoglin polypeptides), and in these embodiments, the polypeptide portion (e.g. BMP10 propeptide polypeptide) is connected to the heterologous portion (e.g., Fc portion) by means of a linker. In some embodiments, the linkers are glycine and serine rich linkers. Other near neutral amino acids, such as, but not limited to, Thr, Asn, Pro and Ala, may also be used in the linker sequence. In some embodiments, the linker comprises various permutations of amino acid sequences containing Gly and Ser. In some embodiments, the linker is greater than 10 amino acids in length. In further embodiments, the linkers have a length of at least 12, 15, 20, 21, 25, 30, 35, 40, 45 or 50 amino acids. In some embodiments, the linker is less than 40, 35, 30, 25, 22 or 20 amino acids. In some embodiments, the linker is 10-50, 10-40, 10-30, 10-25, 10-21, 10-15, 10, 15-25, 17-22, 20, or 21 amino acids in length. In preferred embodiments, the linker comprises the amino acid sequence GlyGlyGlyGlySer (GGGGS) (SEQ ID NO: 34), or repetitions thereof (GGGGS)n, where n ≥ 2. In particular embodiments n ≥ 3, or n = 3-10. In some embodiments, n ≥ 4, or n = 4-10. In some embodiments, n is not greater than 4 in a (GGGGS)n linker. In some embodiments, n = 4-10, 4-9, 4-8, 4-7, 4-6, 4-5, 5-8, 5-7, or 5-6. In some embodiments, n = 3, 4, 5, 6, or 7. In particular embodiments, n = 4. In some embodiments, a linker comprising a (GGGGS)ₙ sequence also comprises an N-terminal threonine. In some embodiments, the linker is any one of the following:

GGGGSGGGGS (SEQ ID NO: 35)
TGGGGSGGGGS (SEQ ID NO: 36)
TGGGGSGGGGSGGGGS (SEQ ID NO: 37)
TGGGGSGGGGSGGGGSGGGGS (SEQ ID NO: 38)
TGGGGSGGGGSGGGGSGGGGSGGGGS (SEQ ID NO: 39)
TGGGGSGGGGSGGGGSGGGGSGGGGSGGGGS (SEQ ID NO: 40) or
TGGGGSGGGGSGGGGSGGGGSGGGGSGGGGSGGGGS (SEQ ID NO: 41).

In some embodiments, the linker comprises the amino acid sequence of TGGGPKSCDK (SEQ ID NO: 42). In some embodiments, the linker is any one of SEQ ID NOs: 35-42 lacking the N-terminal threonine. In some embodiments, the linker does not comprise the amino acid sequence of SEQ ID NO: 40 or 41.

### 4. Nucleic Acids Encoding BMP10 Propeptide Polypeptide, BMPRII, ALK1 Polypeptides, and Endoglin Polypeptides

In certain embodiments, the present disclosure provides isolated and/or recombinant nucleic acids encoding BMPRII, ALK1, endoglin and/or BMP10 propeptide polypeptides (including fragments, functional variants, and fusion proteins thereof) disclosed herein. For example, SEQ ID NO: 7 encodes a naturally occurring human BMPRII precursor polypeptide, SEQ ID NO: 8 encodes a processed extracellular domain of BMPRII. The subject nucleic acids may be single-stranded or double stranded. Such nucleic acids may be DNA or RNA molecules. These nucleic acids may be used, for example, in methods for making BMPRII, ALK1, endoglin and/or BMP 10 propeptide polypeptides as described herein.

As used herein, isolated nucleic acid(s) refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

In certain embodiments, nucleic acids encoding BMPRII, ALK1, endoglin and/or BMP10 propeptide polypeptides of the present disclosure are understood to include any one of SEQ ID NOs: 2, 4, 7, 8, 10, 13, 14, 16, 45, 47, 53, 57, and 60 as well as variants thereof. Variant nucleotide sequences include sequences that differ by one or more nucleotide substitutions, additions, or deletions including allelic variants, and therefore, will include coding sequences that differ from the nucleotide sequence designated in any one of SEQ ID NOs: 2, 4, 7, 8, 10, 13, 14, 16, 45, 47, 53, 57, and 60.

In certain embodiments, BMPRII, ALK1, endoglin and/or BMP10 propeptide polypeptides of the present disclosure are encoded by isolated or recombinant nucleic acid sequences that comprise, consist essentially of, or consists of a sequence that is least 70%, 75%, 80%, 85%, 86%, 87%, 88%, 89%, 90%, 91%, 92%, 93%, 94%, 95%, 96%, 97%, 98%, 99% or 100% identical to SEQ ID NOs: 2, 4, 7, 8, 10, 13, 14, 16, 45, 47, 53, 57, and 60. One of ordinary skill in the art will appreciate that nucleic acid sequences that comprise, consist essentially of, or consists of a sequence complementary to a sequence that is least 70%, 75%, 80%, 85%, 86%, 87%, 88%, 89%, 90%, 91%, 92%, 93%, 94%, 95%, 96%, 97%, 98%, 99% or 100% identical to SEQ ID NOs: 2, 4, 7, 8, 10, 13, 14, 16, 45, 47, 53, 57, and 60 are also within the scope of the present disclosure. In further embodiments, the nucleic acid sequences of the disclosure can be isolated, recombinant, and/or fused with a heterologous nucleotide sequence or in a DNA library.

In other embodiments, nucleic acids of the present disclosure also include nucleotide sequences that hybridize under stringent conditions to the nucleotide sequence designated in SEQ ID NOs: 2, 4, 7, 8, 10, 13, 14, 16, 45, 47, 53, 57, and 60, or fragments thereof. One of ordinary skill in the art will understand readily that appropriate stringency conditions which promote DNA hybridization can be varied. For example, one could perform the hybridization at 6.0 × sodium chloride/sodium citrate (SSC) at about 45° C., followed by a wash of 2.0 × SSC at 50° C. For example, the salt concentration in the wash step can be selected from a low stringency of about 2.0 × SSC at 50° C. to a high stringency of about 0.2 x SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6 × SSC at room temperature followed by a wash at 2 × SSC at room temperature.

Isolated nucleic acids which differ from the nucleic acids as set forth in SEQ ID NOs: 2, 4, 7, 8, 10, 13, 14, 16, 45, 47, 53, 57, and 60 to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the present disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and will vary with the host cell used.

In certain aspects of the present disclosure, the subject nucleic acid is provided in an expression vector comprising a nucleotide sequence encoding an BMPRII, ALK1, endoglin and/or BMP 10 propeptide polypeptide and operably linked to at least one regulatory sequence. Regulatory sequences are art-recognized and are selected to direct expression of BMPRII, ALK1, endoglin and/or BMP10 propeptide polypeptides. Accordingly, the term regulatory sequence includes promoters, enhancers, and other expression control elements. Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding a BMPRII, ALK1, endoglin and/or BMP10 propeptide polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector’s copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant BMPRII, ALK1, endoglin and/or BMP10 propeptide polypeptides include plasmids and other vectors. For instance, suitable vectors include plasmids of the following types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures [Molecular Cloning A Laboratory Manual, 3rd Ed., ed. by Sambrook, Fritsch and Maniatis Cold Spring Harbor Laboratory Press, 2001]. In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In a preferred embodiment, a vector will be designed for production of the subject polypeptides in CHO cells, such as a Pcmv-Script vector (Stratagene, La Jolla, Calif.), pcDNA4 vectors (Invitrogen, Carlsbad, Calif.) and pCI-neo vectors (Promega, Madison, Wisc.). As will be apparent, the subject gene constructs can be used to cause expression of the subject BMPRII, ALK1, endoglin and/or BMP10 propeptide polypeptide in cells propagated in culture, e.g., to produce proteins, including fusion proteins or variant proteins, for purification.

This disclosure also pertains to a host cell transfected with a recombinant gene including a coding sequence for one or more of the subject BMPRII, ALK1, endoglin and/or BMP10 propeptide polypeptides. The host cell may be any prokaryotic or eukaryotic cell. For example, a BMPRII, ALK1, endoglin and/or BMP10 propeptide polypeptide may be expressed in bacterial cells such as E. coli, insect cells (e.g., using a baculovirus expression system), yeast, or mammalian cells [e.g. a Chinese hamster ovary (CHO) cell line]. Other suitable host cells are known to those skilled in the art.

Accordingly, the present disclosure further pertains to methods of producing the subject BMPRII, ALK1, endoglin and/or BMP10 propeptide polypeptides. For example, a host cell transfected with an expression vector encoding a BMPRII, ALK1, endoglin and/or BMP10 propeptide polypeptide can be cultured under appropriate conditions to allow expression of the BMPRII, ALK1, endoglin and/or BMP10 propeptide polypeptide to occur. The polypeptide may be secreted and isolated from a mixture of cells and medium containing the polypeptide. Alternatively, BMPRII, ALK1, endoglin and/or BMP10 propeptide polypeptide may be isolated from a cytoplasmic or membrane fraction obtained from harvested and lysed cells. A cell culture includes host cells, media and other byproducts. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of BMPRII, ALK1, endoglin and/or BMP10 propeptide polypeptides and affinity purification with an agent that binds to a domain fused to BMPRII, ALK1, endoglin and/or BMP10 propeptide polypeptide (e.g., a protein A column may be used to purify BMPRII-Fc, ALK1-Fc, endoglin-Fc and/or BMP10 propeptide —Fc fusion proteins). In some embodiments, the BMPRII, ALK1, endoglin and/or BMP10 propeptide polypeptide is a fusion protein containing a domain which facilitates its purification.

In some embodiments, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. An BMPRII-Fc, ALK1-Fc, endoglin-Fc and/or BMP10 propeptide-Fc fusion protein may be purified to a purity of >90%, >95%, >96%, >98%, or >99% as determined by size exclusion chromatography and >90%, >95%, >96%, >98%, or >99% as determined by SDS PAGE. The target level of purity should be one that is sufficient to achieve desirable results in mammalian systems, particularly non-human primates, rodents (mice), and humans.

In another embodiment, a fusion gene coding for a purification leader sequence, such as a poly-(His)/enterokinase cleavage site sequence at the N-terminus of the desired portion of the recombinant BMPRII, ALK1, endoglin and/or BMP10 propeptide polypeptide, can allow purification of the expressed fusion protein by affinity chromatography using a Ni²⁺ metal resin. The purification leader sequence can then be subsequently removed by treatment with enterokinase to provide the purified BMPRII, ALK1, endoglin and/or BMP10 propeptide polypeptide[Hochuli et al. (1987) J. Chromatography 411:177; and Janknecht et al. (1991) PNAS USA 88:8972].

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence. See, e.g., Current Protocols in Molecular Biology, eds. Ausubel et al., John Wiley & Sons: 1992.

### 5. Antibody Antagonists

In other aspects, the present disclosure relates to a BMP antagonist (inhibitor) that is antibody, or combination of antibodies. A BMP antagonist antibody, or combination of antibodies, may bind to, for example, BMP10, BMP9, BMP6, BMP5, and/or BMP3b or one or more BMP-interacting receptors [e.g., BMPRII, ALK1, and endoglin]. In particular, the disclosure provides methods of using an BMP antagonist antibody, or a combination of BMP antagonist antibodies, alone or in combination with one or more additional supportive therapies and/or active agents, to achieve a desired effect in a subject in need thereof (e.g., treating, preventing, or reducing the progression rate and/or severity of pulmonary hypertension or one or more complications of pulmonary hypertension).

In certain aspects, a BMP antagonist antibody, or combination of antibodies, of the disclosure is an antibody that inhibits at least BMP10. Therefore, in some embodiments, a BMP antagonist antibody, or combination of antibodies, binds to at least BMP10. As used herein, a BMP10 antibody (anti-BMP10 antibody) generally refers to an antibody that binds to BMP10 with sufficient affinity such that the antibody is useful as a diagnostic and/or therapeutic agent in targeting BMP10. In certain embodiments, the extent of binding of an anti-BMP10 antibody to an unrelated, non-BMP10 protein is less than about 10%, 9%, 8%, 7%, 6%, 5%, 4%, 3%, 2%, or less than about 1% of the binding of the antibody to BMP10 as measured, for example, by a radioimmunoassay (RIA), Biacore, or other protein-protein interaction or binding affinity assay. In certain embodiments, an anti-BMP10 antibody binds to an epitope of BMP10 that is conserved among BMP10 from different species. In certain preferred embodiments, an anti-BMP10 antibody binds to human BMP10. In other preferred embodiments, an anti-BMP10 antibody may inhibit BMP10 from binding to BMPRII, ALK1, and/or endoglin and thus inhibit BMP 10-mediated signaling (e.g., Smad signaling) via these receptors. It should be noted that BMP10 has some sequence homology to BMP9 and therefore antibodies that bind to BMP10, in some cases, may also bind to and/or inhibit BMP9. In some embodiments, an anti-BMP10 antibody is a multispecific antibody (e.g., bi-specific antibody) that binds to one or more additional ligands (e.g., BMP9, BMP6, BMP5, and BMP3b) and/or binds to one or more of BMPRII, ALK1, and endoglin. In some embodiments, a BMP10 antibody further binds to BMP9. In some embodiments, the disclosure relates to combinations of antibodies, as well as uses thereof, wherein the combination of antibodies comprises an anti-BMP10 antibody and one or more additional antibodies that bind to, for example, different ligands (e.g., BMP9, BMP6, BMP5, and BMP3b) and/or binds to one or more of BMPRII, ALK1, and endoglin. In some embodiments, a combination antibodies comprising an anti-BMP10 antibody further comprises an anti-BMP9 antibody. Preferably, BMP10 antibodies bind to the mature BMP10 domain and bind competitively with a BMP10 propeptide.

In certain aspects, a BMP antagonist antibody, or combination of antibodies, of the disclosure is an antibody that inhibits at least BMP9. Therefore, in some embodiments, a BMP antagonist antibody, or combination of antibodies, binds to at least BMP9. As used herein, a BMP9 antibody (anti-BMP9 antibody) generally refers to an antibody that binds to BMP9 with sufficient affinity such that the antibody is useful as a diagnostic and/or therapeutic agent in targeting BMP9. In certain embodiments, the extent of binding of an anti-BMP9 antibody to an unrelated, non-BMP9 protein is less than about 10%, 9%, 8%, 7%, 6%, 5%, 4%, 3%, 2%, or less than about 1% of the binding of the antibody to BMP9 as measured, for example, by a radioimmunoassay (RIA), Biacore, or other protein-protein interaction or binding affinity assay. In certain embodiments, an anti-BMP9 antibody binds to an epitope of BMP9 that is conserved among BMP9 from different species. In certain preferred embodiments, an anti-BMP9 antibody binds to human BMP9. In other preferred embodiments, an anti-BMP9 antibody may inhibit BMP9 from binding to BMPRII, ALK1, and/or endoglin and thus inhibit BMP9-mediated signaling (e.g., Smad signaling) via these receptors. It should be noted that BMP9 has some sequence homology to BMP10 and therefore antibodies that bind to BMP9, in some cases, may also bind to and/or inhibit BMP10. In some embodiments, an anti-BMP9 antibody is a multispecific antibody (e.g., bi-specific antibody) that binds to one or more additional ligands [e.g., BMP10, BMP6, BMP5, and BMP3b] and/or binds to one or more of BMPRII, ALK1, and/or endoglin. In some embodiments, a BMP9 antibody further binds to BMP10. In some embodiments, the disclosure relates to combinations of antibodies, as well as uses thereof, wherein the combination of antibodies comprises an anti-BMP9 antibody and one or more additional antibodies that bind to, for example, different ligands (e.g., BMP10, BMP6, BMP5, and BMP3b) and/or binds to one or more of BMPRII, ALK1, and/or endoglin. In some embodiments, a combination antibodies comprising an anti-BMP9 antibody further comprises an anti-BMP10 antibody.

In certain aspects, a BMP antagonist antibody, or combination of antibodies, of the disclosure is an antibody that inhibits at least BMP6. Therefore, in some embodiments, a BMP antagonist antibody, or combination of antibodies, binds to at least BMP6. As used herein, a BMP6 antibody (anti-BMP6 antibody) generally refers to an antibody that binds to BMP6 with sufficient affinity such that the antibody is useful as a diagnostic and/or therapeutic agent in targeting BMP6. In certain embodiments, the extent of binding of an anti-BMP6 antibody to an unrelated, non-BMP6 protein is less than about 10%, 9%, 8%, 7%, 6%, 5%, 4%, 3%, 2%, or less than about 1% of the binding of the antibody to BMP6 as measured, for example, by a radioimmunoassay (RIA), Biacore, or other protein-protein interaction or binding affinity assay. In certain embodiments, an anti-BMP6 antibody binds to an epitope of BMP6 that is conserved among BMP6 from different species. In certain preferred embodiments, an anti-BMP6 antibody binds to human BMP6. In other preferred embodiments, an anti-BMP6 antibody may inhibit BMP6 from binding to BMPRII, ALK1, and/or endoglin and thus inhibit BMP6-mediated signaling (e.g., Smad signaling) via these receptors. In some embodiments, an anti-BMP6 antibody is a multispecific antibody (e.g., bi-specific antibody) that binds to one or more additional ligands (e.g., BMP10, BMP9, BMP5, and BMP3b) and/or binds to one or more of BMPRII, ALK1, and endoglin. In some embodiments, a BMP6 antibody further binds to BMP9 and/or BMP10. In some embodiments, the disclosure relates to combinations of antibodies, as well as uses thereof, wherein the combination of antibodies comprises an anti-BMP6 antibody and one or more additional antibodies that bind to, for example, different ligands (e.g., BMP10, BMP9, BMP5, and BMP3b) and/or binds to one or more of BMPRII, ALK1, and endoglin. In some embodiments, a combination antibodies comprising an anti-BMP6 antibody further comprises an anti-BMP10 and/or BMP9 antibody.

In certain aspects, a BMP antagonist antibody, or combination of antibodies, of the disclosure is an antibody that inhibits at least BMP5. Therefore, in some embodiments, a BMP antagonist antibody, or combination of antibodies, binds to at least BMP5. As used herein, a BMP5 antibody (anti-BMP5 antibody) generally refers to an antibody that binds to BMP5 with sufficient affinity such that the antibody is useful as a diagnostic and/or therapeutic agent in targeting BMP5. In certain embodiments, the extent of binding of an anti-BMP5 antibody to an unrelated, non-BMP5 protein is less than about 10%, 9%, 8%, 7%, 6%, 5%, 4%, 3%, 2%, or less than about 1% of the binding of the antibody to BMP5 as measured, for example, by a radioimmunoassay (RIA), Biacore, or other protein-protein interaction or binding affinity assay. In certain embodiments, an anti-BMP5 antibody binds to an epitope of BMP5 that is conserved among BMP5 from different species. In certain preferred embodiments, an anti-BMP5 antibody binds to human BMP5. In other preferred embodiments, an anti-BMP5 antibody may inhibit BMP5 from binding to BMPRII, ALK1, and/or endoglin and thus inhibit BMP5-mediated signaling (e.g., Smad signaling) via these receptors. In some embodiments, an anti-BMP5 antibody is a multispecific antibody (e.g., bi-specific antibody) that binds to one or more additional ligands (e.g., BMP10, BMP9, BMP6, and BMP3b) and/or binds to one or more of BMPRII, ALK1, and endoglin. In some embodiments, a BMP5 antibody further binds to BMP9 and/or BMP10. In some embodiments, the disclosure relates to combinations of antibodies, as well as uses thereof, wherein the combination of antibodies comprises an anti-BMP5 antibody and one or more additional antibodies that bind to, for example, different ligands (e.g., BMP10, BMP9, BMP6, and BMP3b) and/or binds to one or more of BMPRII, ALK1, and endoglin. In some embodiments, a combination antibodies comprising an anti-BMP5 antibody further comprises an anti-BMP10 and/or BMP9 antibody.

In certain aspects, a BMP antagonist antibody, or combination of antibodies, of the disclosure is an antibody that inhibits at least BMP3b. Therefore, in some embodiments, a BMP antagonist antibody, or combination of antibodies, binds to at least BMP3b. As used herein, a BMP3b antibody (anti-BMP3b antibody) generally refers to an antibody that binds to BMP3b with sufficient affinity such that the antibody is useful as a diagnostic and/or therapeutic agent in targeting BMP3b. In certain embodiments, the extent of binding of an anti-BMP3b antibody to an unrelated, non-BMP3b protein is less than about 10%, 9%, 8%, 7%, 6%, 5%, 4%, 3%, 2%, or less than about 1% of the binding of the antibody to BMP3b as measured, for example, by a radioimmunoassay (RIA), Biacore, or other protein-protein interaction or binding affinity assay. In certain embodiments, an anti-BMP3b antibody binds to an epitope of BMP3b that is conserved among BMP3b from different species. In certain preferred embodiments, an anti-BMP3b antibody binds to human BMP3b. In other preferred embodiments, an anti-BMP3b antibody may inhibit BMP3b from binding to BMPRII, ALK1, and/or endoglin and thus inhibit BMP3b-mediated signaling (e.g., Smad signaling) via these receptors. In some embodiments, an anti-BMP3b antibody is a multispecific antibody (e.g., bi-specific antibody) that binds to one or more additional ligands (e.g., BMP10, BMP9, BMP6, and BMP5) and/or binds to one or more of BMPRII, ALK1, and endoglin. In some embodiments, a BMP3b antibody further binds to BMP9 and/or BMP10. In some embodiments, the disclosure relates to combinations of antibodies, as well as uses thereof, wherein the combination of antibodies comprises an anti-BMP3b antibody and one or more additional antibodies that bind to, for example, different ligands (e.g., BMP10, BMP9, BMP6, and BMP5) and/or binds to one or more of BMPRII, ALK1, and endoglin. In some embodiments, a combination antibodies comprising an anti-BMP3b antibody further comprises an anti-BMP10 and/or BMP9 antibody.

In other aspects, a BMP antagonist antibody, or combination of antibodies, of the disclosure is an antibody that inhibits at least ALK1. Therefore, in some embodiments, a BMP antagonist antibody, or combination of antibodies, binds to at least ALK1. As used herein, an ALK1 antibody (anti-ALK1 antibody) generally refers to an antibody that binds to ALK1 with sufficient affinity such that the antibody is useful as a diagnostic and/or therapeutic agent in targeting ALK1. In certain embodiments, the extent of binding of an anti-ALK1 antibody to an unrelated, non-ALK1 protein is less than about 10%, 9%, 8%, 7%, 6%, 5%, 4%, 3%, 2%, or less than about 1% of the binding of the antibody to ALK1 as measured, for example, by a radioimmunoassay (RIA), Biacore, or other protein-protein interaction or binding affinity assay. In certain embodiments, an anti-ALK1 antibody binds to an epitope of ALK1 that is conserved among ALK1 from different species. In certain preferred embodiments, an anti-ALK1 antibody binds to human ALK1. In other preferred embodiments, an anti-ALK1 antibody may inhibit one or more ligands (e.g., BMP10 and BMP9) from binding to ALK1. In some embodiments, an anti-ALK1 antibody is a multispecific antibody (e.g., bi-specific antibody) that binds to ALK1 and one or more ligands (e.g. BMP9 and BMP10), BMPRII, and/or endoglin. In some embodiments, the disclosure relates to combinations of antibodies, as well as uses thereof, wherein the combination of antibodies comprises an anti-ALK1 antibody and one or more additional antibodies that bind to, for example, one or more ligands (e.g. BMP9 and BMP10), BMPRII, and/or endoglin.

In other aspects, a BMP antagonist antibody, or combination of antibodies, of the disclosure is an antibody that inhibits at least BMPRII. Therefore, in some embodiments, a BMP antagonist antibody, or combination of antibodies, binds to at least BMPRII. As used herein, an BMPRII antibody (anti-BMPRII antibody) generally refers to an antibody that binds to BMPRII with sufficient affinity such that the antibody is useful as a diagnostic and/or therapeutic agent in targeting BMPRII. In certain embodiments, the extent of binding of an anti-BMPRII antibody to an unrelated, non-BMPRII protein is less than about 10%, 9%, 8%, 7%, 6%, 5%, 4%, 3%, 2%, or less than about 1% of the binding of the antibody to BMPRII as measured, for example, by a radioimmunoassay (RIA), Biacore, or other protein-protein interaction or binding affinity assay. In certain embodiments, an anti-BMPRII antibody binds to an epitope of BMPRII that is conserved among BMPRII from different species. In certain preferred embodiments, an anti-BMPRII antibody binds to human BMPRII. In other preferred embodiments, an anti-BMPRII antibody may inhibit one or more ligands (e.g., BMP10 and BMP9) from binding to BMPRII. In some embodiments, an anti-BMPRII antibody is a multispecific antibody (e.g., bi-specific antibody) that binds to BMPRII and one or more ligands (e.g. BMP9 and BMP10), ALK1, and/or endoglin. In some embodiments, the disclosure relates to combinations of antibodies, as well as uses thereof, wherein the combination of antibodies comprises an anti-BMPRII antibody and one or more additional antibodies that bind to, for example, one or more ligands (e.g. BMP9 and BMP10), ALK1, and/or endoglin.

In other aspects, a BMP antagonist antibody, or combination of antibodies, of the disclosure is an antibody that inhibits at least endoglin. Therefore, in some embodiments, a BMP antagonist antibody, or combination of antibodies, binds to at least endoglin. As used herein, a endoglin antibody (anti-endoglin antibody) generally refers to an antibody that binds to endoglin with sufficient affinity such that the antibody is useful as a diagnostic and/or therapeutic agent in targeting endoglin. In certain embodiments, the extent of binding of an anti-endoglin antibody to an unrelated, non-endoglin protein is less than about 10%, 9%, 8%, 7%, 6%, 5%, 4%, 3%, 2%, or less than about 1% of the binding of the antibody to endoglin as measured, for example, by a radioimmunoassay (RIA), Biacore, or other protein-protein interaction or binding affinity assay. In certain embodiments, an anti-endoglin antibody binds to an epitope of endoglin that is conserved among endoglin from different species. In certain preferred embodiments, an anti-endoglin antibody binds to human endoglin. In other preferred embodiments, an anti-endoglin antibody may inhibit one or more ligands (e.g., BMP10 and BMP9) from binding to endoglin. In some embodiments, an anti-endoglin antibody is a multispecific antibody (e.g., bi-specific antibody) that binds to endoglin and one or more ligands (e.g. BMP9 and BMP10), ALK1, and/or BMPRII. In some embodiments, the disclosure relates to combinations of antibodies, as well as uses thereof, wherein the combination of antibodies comprises an anti-endoglin antibody and one or more additional antibodies that bind to, for example, one or more ligands (e.g. BMP9 and BMP10), ALK1, and/or BMPRII.

The term antibody is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody fragment refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments. See, e.g., Hudson et al. (2003) Nat. Med. 9:129-134; Plückthun, in The Pharmacology of Monoclonal Antibodies, vol. 113, Rosenburg and Moore eds., (Springer-Verlag, New York), pp. 269-315 (1994); WO 93/16185; and U.S. Pat. Nos. 5,571,894, 5,587,458, and 5,869,046. Antibodies disclosed herein may be polyclonal antibodies or monoclonal antibodies. In certain embodiments, the antibodies of the present disclosure comprise a label attached thereto and able to be detected (e.g., the label can be a radioisotope, fluorescent compound, enzyme, or enzyme co-factor). In preferred embodiments, the antibodies of the present disclosure are isolated antibodies.

Diabodies are antibody fragments with two antigen-binding sites that may be bivalent or bispecific. See, e.g., EP 404,097; WO 1993/01161; Hudson et al. (2003) Nat. Med. 9:129-134 (2003); and Hollinger et al. (1993) Proc. Natl. Acad. Sci. USA 90: 6444-6448. Triabodies and tetrabodies are also described in Hudson et al. (2003) Nat. Med. 9:129-134.

Single-domain antibodies are antibody fragments comprising all or a portion of the heavy-chain variable domain or all or a portion of the light-chain variable domain of an antibody. In certain embodiments, a single-domain antibody is a human single-domain antibody. See, e.g., U.S. Pat. No. 6,248,516.

Antibody fragments can be made by various techniques, including but not limited to proteolytic digestion of an intact antibody as well as production by recombinant host cells (e.g., E. coli or phage), as described herein.

The antibodies herein may be of any class. The class of an antibody refers to the type of constant domain or constant region possessed by its heavy chain. There are five major classes of antibodies: IgA, IgD, IgE, IgG, and IgM, and several of these may be further divided into subclasses (isotypes), for example, IgG₁, IgG₂, IgG₃, IgG₄, IgA₁, and IgA₂. The heavy-chain constant domains that correspond to the different classes of immunoglobulins are called alpha, delta, epsilon, gamma, and mu.

In general, an antibody for use in the methods disclosed herein specifically binds to its target antigen, preferably with high binding affinity. Affinity may be expressed as a K_{D} value and reflects the intrinsic binding affinity (e.g., with minimized avidity effects). Typically, binding affinity is measured in vitro, whether in a cell-free or cell-associated setting. Any of a number of assays known in the art, including those disclosed herein, can be used to obtain binding affinity measurements including, for example, surface plasmon resonance (Biacore™ assay), radiolabeled antigen binding assay (RIA), and ELISA. In some embodiments, antibodies of the present disclosure bind to their target antigens [e.g., BMP10, BMP9, BMP6, BMP5, BMP3b, BMPRII, ALK1, and endoglin] with at least a K_{D} of 1× 10⁻⁷ or stronger, 1×10⁻⁸ or stronger, 1×10⁻⁹ or stronger, 1×10⁻¹⁰ or stronger, 1×10⁻¹¹ or stronger, 1×10⁻¹² or stronger, 1×10⁻¹³ or stronger, or 1×10⁻¹⁴ or stronger.

In certain embodiments, K_{D} is measured by RIA performed with the Fab version of an antibody of interest and its target antigen as described by the following assay. Solution binding affinity of Fabs for the antigen is measured by equilibrating Fab with a minimal concentration of radiolabeled antigen (e.g., ¹²⁵I-labeled) in the presence of a titration series of unlabeled antigen, then capturing bound antigen with an anti-Fab antibody-coated plate [see, e.g., Chen et al. (1999) J. Mol. Biol. 293:865-881]. To establish conditions for the assay, multi-well plates (e.g., MICROTITER® from Thermo Scientific) are coated (e.g., overnight) with a capturing anti-Fab antibody (e.g., from Cappel Labs) and subsequently blocked with bovine serum albumin, preferably at room temperature (e.g., approximately 23° C.). In a non-adsorbent plate, radiolabeled antigen are mixed with serial dilutions of a Fab of interest [e.g., consistent with assessment of the anti-VEGF antibody, Fab-12, in Presta et al., (1997) Cancer Res. 57:4593-4599]. The Fab of interest is then incubated, preferably overnight but the incubation may continue for a longer period (e.g., about 65 hours) to ensure that equilibrium is reached. Thereafter, the mixtures are transferred to the capture plate for incubation, preferably at room temperature for about one hour. The solution is then removed and the plate is washed times several times, preferably with polysorbate 20 and PBS mixture. When the plates have dried, scintillant (e.g., MICROSCINT® from Packard) is added, and the plates are counted on a gamma counter (e.g., TOPCOUNT® from Packard).

According to another embodiment, K_{D} is measured using surface plasmon resonance assays using, for example a BIACORE® 2000 or a BIACORE® 3000 (Biacore, Inc., Piscataway, N.J.) with immobilized antigen CM5 chips at about 10 response units (RU). Briefly, carboxymethylated dextran biosensor chips (CM5, Biacore, Inc.) are activated with N-ethyl-N′-(3-dimethylaminopropyl)-carbodiimide hydrochloride (EDC) and N-hydroxysuccinimide (NHS) according to the supplier’s instructions. For example, an antigen can be diluted with 10 mM sodium acetate, pH 4.8, to 5 µg/ml (about 0.2 µM) before injection at a flow rate of 5 µl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20®) surfactant (PBST) at at a flow rate of approximately 25 µl/min. Association rates (kₒₙ) and dissociation rates (k_{off}) are calculated using, for example, a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off} / kₒₙ [see, e.g., Chen et al., (1999) J. Mol. Biol. 293:865-881]. If the on-rate exceeds, for example, 10⁶ M⁻¹ s⁻¹ by the surface plasmon resonance assay above, then the on-rate can be determined by using a fluorescent quenching technique that measures the increase or decrease in fluorescence emission intensity (e.g., excitation=295 nm; emission=340 nm, 16 nm bandpass) of a 20 nM anti-antigen antibody (Fab form) in PBS in the presence of increasing concentrations of antigen as measured in a spectrometer, such as a stop-flow equipped spectrophometer (Aviv Instruments) or a 8000-series SLM-AMINCO® spectrophotometer (ThermoSpectronic) with a stirred cuvette.

The nucleic acid and amino acid sequences of human BMP10, BMP9, BMP6, BMP5, BMP3b, BMPRII, ALK1, and endoglin are well known in the art and thus antibody antagonists for use in accordance with this disclosure may be routinely made by the skilled artisan based on the knowledge in the art and teachings provided herein.

In certain embodiments, an antibody provided herein is a chimeric antibody. A chimeric antibody refers to an antibody in which a portion of the heavy and/or light chain is derived from a particular source or species, while the remainder of the heavy and/or light chain is derived from a different source or species. Certain chimeric antibodies are described, for example, in U.S. Pat. No. 4,816,567; and Morrison et al., (1984) Proc. Natl. Acad. Sci. USA, 81:6851-6855. In some embodiments, a chimeric antibody comprises a non-human variable region (e.g., a variable region derived from a mouse, rat, hamster, rabbit, or non-human primate, such as a monkey) and a human constant region. In some embodiments, a chimeric antibody is a “class switched” antibody in which the class or subclass has been changed from that of the parent antibody. In general, chimeric antibodies include antigen-binding fragments thereof.

In certain embodiments, a chimeric antibody provided herein is a humanized antibody. A humanized antibody refers to a chimeric antibody comprising amino acid residues from non-human hypervariable regions (HVRs) and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

Humanized antibodies and methods of making them are reviewed, for example, in Almagro and Fransson (2008) Front. Biosci. 13:1619-1633 and are further described, for example, in Riechmann et al., (1988) Nature 332:323-329; Queen et al. (1989) Proc. Nat’l Acad. Sci. USA 86:10029-10033; U.S. Pat. Nos. 5,821,337, 7,527,791, 6,982,321, and 7,087,409; Kashmiri et al., (2005) Methods 36:25-34 [describing SDR (a-CDR) grafting]; Padlan, Mol. Immunol. (1991) 28:489-498 (describing “resurfacing”); Dall’Acqua et al. (2005) Methods 36:43-60 (describing “FR shuffling”); Osbourn et al. (2005) Methods 36:61-68; and Klimka et al. Br. J. Cancer (2000) 83:252-260 (describing the “guided selection” approach to FR shuffling).

Human framework regions that may be used for humanization include but are not limited to: framework regions selected using the “best-fit” method [see, e.g., Sims et al. (1993) J. Immunol. 151:2296 ]; framework regions derived from the consensus sequence of human antibodies of a particular subgroup of light-chain or heavy-chain variable regions [see, e.g., Carter et al. (1992) Proc. Natl. Acad. Sci. USA, 89:4285; and Presta et al. (1993) J. Immunol., 151:2623]; human mature (somatically mutated) framework regions or human germline framework regions [see, e.g., Almagro and Fransson (2008) Front. Biosci. 13:1619-1633]; and framework regions derived from screening FR libraries [see, e.g., Baca et cd., (1997) J. Biol. Chem. 272:10678-10684; and Rosok et cd., (1996) J. Biol. Chem. 271:22611-22618].

In certain embodiments, an antibody provided herein is a human antibody. Human antibodies can be produced using various techniques known in the art. Human antibodies are described generally in van Dijk and van de Winkel (2001) Curr. Opin. Pharmacol. 5: 368-74 and Lonberg (2008) Curr. Opin. Immunol. 20:450-459.

Human antibodies may be prepared by administering an immunogen [e.g., BMP10, BMP9, BMP6, BMP5, BMP3b, BMPRII, ALK1, and endoglin] to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal’s chromosomes. In such transgenic animals, the endogenous immunoglobulin loci have generally been inactivated. For a review of methods for obtaining human antibodies from transgenic animals, see, for example, Lonberg (2005) Nat. Biotechnol. 23:1117-1125; U.S. Pat. Nos. 6,075,181 and 6,150,584 (describing XENOMOUSE™ technology); U.S. Pat. No. 5,770,429 (describing HuMab® technology); U.S. Pat. No. 7,041,870 (describing K-M MOUSE® technology); and U.S. Pat. Application Publication No. 2007/0061900 (describing VelociMouse® technology). Human variable regions from intact antibodies generated by such animals may be further modified, for example, by combining with a different human constant region.

Human antibodies provided herein can also be made by hybridoma-based methods. Human myeloma and mouse-human heteromyeloma cell lines for the production of human monoclonal antibodies have been described [see, e.g., Kozbor J. Immunol., (1984) 133: 3001; Brodeur et al. (1987) Monoclonal Antibody Production Techniques and Applications, pp. 51-63, Marcel Dekker, Inc., New York; and Boerner et al. (1991) J. Immunol., 147: 86]. Human antibodies generated via human B-cell hybridoma technology are also described in Li et al., (2006) Proc. Natl. Acad. Sci. USA, 103:3557-3562. Additional methods include those described, for example, in U.S. Pat. No. 7,189,826 (describing production of monoclonal human IgM antibodies from hybridoma cell lines) and Ni, Xiandai Mianyixue (2006) 26(4):265-268 (2006) (describing human-human hybridomas). Human hybridoma technology (Trioma technology) is also described in Vollmers and Brandlein (2005) Histol. Histopathol., 20(3):927-937 (2005) and Vollmers and Brandlein (2005) Methods Find Exp. Clin. Pharmacol., 27(3):185-91.

Human antibodies provided herein may also be generated by isolating Fv clone variable-domain sequences selected from human-derived phage display libraries. Such variable-domain sequences may then be combined with a desired human constant domain. Techniques for selecting human antibodies from antibody libraries are described herein.

For example, antibodies of the present disclosure may be isolated by screening combinatorial libraries for antibodies with the desired activity or activities. A variety of methods are known in the art for generating phage-display libraries and screening such libraries for antibodies possessing the desired binding characteristics. Such methods are reviewed, for example, in Hoogenboom et al. (2001) in Methods in Molecular Biology 178:1-37, O’Brien et al., ed., Human Press, Totowa, N.J. and further described, for example, in the McCafferty et al. (1991) Nature 348:552-554; Clackson et al., (1991) Nature 352: 624-628; Marks et al. (1992) J. Mol. Biol. 222:581-597; Marks and Bradbury (2003) in Methods in Molecular Biology 248:161-175, Lo, ed., Human Press, Totowa, N.J.; Sidhu et al. (2004) J. Mol. Biol. 338(2):299-310; Lee et al. (2004) J. Mol. Biol. 340(5):1073-1093; Fellouse (2004) Proc. Natl. Acad. Sci. USA 101(34):12467-12472; and Lee et al. (2004) J. Immunol. Methods 284(1-2): 119-132.

In certain phage display methods, repertoires of VH and VL genes are separately cloned by polymerase chain reaction (PCR) and recombined randomly in phage libraries, which can then be screened for antigen-binding phage as described in Winter et al. (1994) Ann. Rev. Immunol., 12: 433-455. Phage typically display antibody fragments, either as single-chain Fv (scFv) fragments or as Fab fragments. Libraries from immunized sources provide high-affinity antibodies to the immunogen [e.g., BMP10, BMP9, BMP6, BMP5, BMP3b, BMPRII, ALK1, and endoglin] without the requirement of constructing hybridomas. Alternatively, the naive repertoire can be cloned (e.g., from human) to provide a single source of antibodies directed against a wide range of non-self and also self-antigens without any immunization as described by Griffiths et al. (1993) EMBO J, 12: 725-734. Finally, naive libraries can also be made synthetically by cloning un-rearranged V-gene segments from stem cells and using PCR primers containing random sequence to encode the highly variable CDR3 regions and to accomplish rearrangement in vitro, as described by Hoogenboom and Winter (1992) J. Mol. Biol., 227: 381-388. Patent publications describing human antibody phage libraries include, for example: U.S. Pat. No. 5,750,373, and U.S. Pat. Publication Nos. 2005/0079574, 2005/0119455, 2005/0266000, 2007/0117126, 2007/0160598, 2007/0237764, 2007/0292936, and 2009/0002360.

In certain embodiments, an antibody provided herein is a multispecific antibody, for example, a bispecific antibody. Multispecific antibodies (typically monoclonal antibodies) have binding specificities for at least two different epitopes (e.g., two, three, four, five, or six or more) on one or more (e.g., two, three, four, five, six or more) antigens.

Engineered antibodies with three or more functional antigen binding sites, including “octopus antibodies,” are also included herein (see, e.g., US 2006/0025576A1).

In certain embodiments, the antibodies disclosed herein are monoclonal antibodies. Monoclonal antibody refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present methods may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

For example, by using immunogens derived from BMP10, anti-protein/anti-peptide antisera or monoclonal antibodies can be made by standard protocols [see, e.g., Antibodies: A Laboratory Manual (1988) ed. by Harlow and Lane, Cold Spring Harbor Press]. A mammal, such as a mouse, hamster, or rabbit can be immunized with an immunogenic form of the BMP10 polypeptide, an antigenic fragment which is capable of eliciting an antibody response, or a fusion protein. Techniques for conferring immunogenicity on a protein or peptide include conjugation to carriers or other techniques well known in the art. An immunogenic portion of a BMP10 polypeptide can be administered in the presence of adjuvant. The progress of immunization can be monitored by detection of antibody titers in plasma or serum. Standard ELISA or other immunoassays can be used with the immunogen as antigen to assess the levels of antibody production and/or level of binding affinity.

Following immunization of an animal with an antigenic preparation of BMP10, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique [see, e.g., Kohler and Milstein (1975) Nature, 256: 495-497], the human B cell hybridoma technique [see, e.g., Kozbar et al. (1983) Immunology Today, 4:72], and the EBV-hybridoma technique to produce human monoclonal antibodies [Cole et al. (1985) Monoclonal Antibodies and Cancer Therapy, Alan R. Liss, Inc. pp. 77-96]. Hybridoma cells can be screened immunochemically for production of antibodies specifically reactive with a BMP10 polypeptide, and monoclonal antibodies isolated from a culture comprising such hybridoma cells.

In certain embodiments, one or more amino acid modifications may be introduced into the Fc region of an antibody provided herein thereby generating an Fc-region variant. The Fc-region variant may comprise a human Fc-region sequence (e.g., a human IgG1, IgG2, IgG3 or IgG4 Fc region) comprising an amino acid modification (e.g., a substitution, deletion, and/or addition) at one or more amino acid positions.

For example, the present disclosure contemplates an antibody variant that possesses some but not all effector functions, which make it a desirable candidate for applications in which the half-life of the antibody in vivo is important yet for which certain effector functions [e.g., complement-dependent cytotoxicity (CDC) and antibody-dependent cellular cytotoxicity (ADCC)] are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcyR binding (hence likely lacking ADCC activity), but retains FcRn binding ability. The primary cells for mediating ADCC, NK cells, express FcyRIII only, whereas monocytes express FcyRI, FcyRII and FcyRIII. FcR expression on hematopoietic cells is summarized in, for example, Ravetch and Kinet (1991) Annu. Rev. Immunol. 9:457-492. Non-limiting examples of in vitro assays to assess ADCC activity of a molecule of interest are described in U.S. Pat. No. 5,500,362; Hellstrom, I. et al. (1986) Proc. Nat’l Acad. Sci. USA 83:7059-7063; Hellstrom, I et al. (1985) Proc. Nat’l Acad. Sci. USA 82:1499-1502; U.S. Pat. No. 5,821,337; and Bruggemann, M. et al. (1987) J. Exp. Med. 166:1351-1361. Alternatively, non-radioactive assay methods may be employed (e.g., ACTI™, non-radioactive cytotoxicity assay for flow cytometry; CellTechnology, Inc. Mountain View, Calif.; and CytoTox 96® non-radioactive cytotoxicity assay, Promega, Madison, Wis.). Useful effector cells for such assays include peripheral blood mononuclear cells (PBMC) and natural killer (NK) cells. Alternatively, or additionally, ADCC activity of the molecule of interest may be assessed in vivo, for example, in an animal model such as that disclosed in Clynes et al. (1998) Proc. Nat’l Acad. Sci. USA 95:652-656. Clq binding assays may also be carried out to confirm that the antibody is unable to bind Clq and hence lacks CDC activity [see, e.g., Clq and C3c binding ELISA in WO 2006/029879 and WO 2005/100402]. To assess complement activation, a CDC assay may be performed [see, e.g., Gazzano-Santoro et al. (1996) J. Immunol. Methods 202:163; Cragg, M. S. et al. (2003) Blood 101:1045-1052; and Cragg, M. S, and M. J. Glennie (2004) Blood 103:2738-2743]. FcRn binding and in vivo clearance/half-life determinations can also be performed using methods known in the art [see, e.g., Petkova, S. B. et al. (2006) Int. Immunol. 18(12):1759-1769].

Antibodies of the present disclosure with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). In some embodiments, antibodies of the present disclosure with reduced effector function include but are not limited to, antibodies comprising the Ala—Ala mutation described by Bluestone et al. (see WO 94/28027 and WO 98/47531; also see Xu et al. 2000 Cell Immunol 200; 16-26) or the P329G/L234A/L235A ( P329G LALA) mutation described by Schlothauer et al. (see Schlothauer T., et al. Protein Eng Des Sel. 2016 Oct;29(10):457-466). Such Fc mutants include Fc mutants comprising mutation(s) from phenylalanine to alanine at position 234; a mutation from leucine to alanine at position 235; and/or a mutation from proline to glycine at position 329.

In certain embodiments, it may be desirable to create cysteine-engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy-chain Fc region. Cysteine engineered antibodies may be generated as described, for example., in U.S. Pat. No. 7,521,541.

In addition, the techniques used to screen antibodies in order to identify a desirable antibody may influence the properties of the antibody obtained. For example, if an antibody is to be used for binding an antigen in solution, it may be desirable to test solution binding. A variety of different techniques are available for testing interaction between antibodies and antigens to identify particularly desirable antibodies. Such techniques include ELISAs, surface plasmon resonance binding assays (e.g., the Biacore™ binding assay, Biacore AB, Uppsala, Sweden), sandwich assays (e.g., the paramagnetic bead system of IGEN International, Inc., Gaithersburg, Maryland), western blots, immunoprecipitation assays, and immunohistochemistry.

In certain embodiments, amino acid sequence variants of the antibodies and/or the binding polypeptides provided herein are contemplated. For example, it may be desirable to improve the binding affinity and/or other biological properties of the antibody and/or binding polypeptide. Amino acid sequence variants of an antibody and/or binding polypeptides may be prepared by introducing appropriate modifications into the nucleotide sequence encoding the antibody and/or binding polypeptide, or by peptide synthesis. Such modifications include, for example, deletions from, and/or insertions into, and/or substitutions of residues within, the amino acid sequences of the antibody and/or binding polypeptide. Any combination of deletion, insertion, and substitution can be made to arrive at the final construct, provided that the final construct possesses the desired characteristics, e.g., target-binding (BMP10, BMP9, BMP6, BMP5, BMP3b, BMPRII, ALK1, and endoglin binding).

Alterations (e.g., substitutions) may be made in HVRs, for example, to improve antibody affinity. Such alterations may be made in HVR “hotspots,” i.e., residues encoded by codons that undergo mutation at high frequency during the somatic maturation process (see, e.g., Chowdhury (2008) Methods Mol. Biol. 207:179-196 (2008)), and/or SDRs (a-CDRs), with the resulting variant VH or VL being tested for binding affinity. Affinity maturation by constructing and reselecting from secondary libraries has been described in the art [see, e.g., Hoogenboom et al., in Methods in Molecular Biology 178:1-37, O’Brien et al., ed., Human Press, Totowa, N.J., (2001)]. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind to the antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two, or three amino acid substitutions.

A useful method for identification of residues or regions of the antibody and/or the binding polypeptide that may be targeted for mutagenesis is called “alanine scanning mutagenesis”, as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody or binding polypeptide with antigen is affected. Further substitutions may be introduced at the amino acid locations demonstrating functional sensitivity to the initial substitutions. Alternatively, or additionally, a crystal structure of an antigen-antibody complex can be used to identify contact points between the antibody and antigen. Such contact residues and neighboring residues may be targeted or eliminated as candidates for substitution. Variants may be screened to determine whether they contain the desired properties.

Amino-acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include fusion of the N- or C-terminus of the antibody to an enzyme (e.g., for ADEPT) or a polypeptide which increases the serum half-life of the antibody.

In certain embodiments, an antibody and/or binding polypeptide provided herein may be further modified to contain additional non-proteinaceous moieties that are known in the art and readily available. The moieties suitable for derivatization of the antibody and/or binding polypeptide include but are not limited to water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody and/or binding polypeptide may vary, and if more than one polymer are attached, they can be the same or different molecules. In general, the number and/or type of polymers used for derivatization can be determined based on considerations including, but not limited to, the particular properties or functions of the antibody and/or binding polypeptide to be improved, whether the antibody derivative and/or binding polypeptide derivative will be used in a therapy under defined conditions.

### 6. Small Molecule Antagonists

In other aspects, the present disclosure relates to a BMP antagonist (inhibitor) that is small molecule, or combination of small molecules. BMP antagonist small molecules may inhibit one or more ligands [e.g., BMP10, BMP9, BMP6, BMP5, and BMP3b] and/or one or more of BMPRII, ALK1, and endoglin, and/or one or more downstream signaling components (e.g., Smad proteins). In particular, the disclosure provides methods of using an BMP antagonist small molecule, or combination of BMP antagonist small molecules, alone or in combination with one or more additional supportive therapies and/or active agents, to achieve a desired effect in a subject in need thereof (e.g., treating, preventing, or reducing the progression rate and/or severity of pulmonary hypertension or one or more complications of pulmonary hypertension).

In some embodiments, a BMP antagonist is a small molecule antagonist, or combination of small molecule antagonists, that inhibits at least BMP10. In some embodiments, a small molecule antagonist, or combination of small molecule antagonists, that inhibits BMP10 further inhibits one or more ligands [e.g., BMP9, BMP6, BMP5 and BMP3b], BMPRII, ALK1, endoglin, and/or one or more Smads. In some embodiments, a BMP antagonist is a small molecule antagonist, or combination of small molecule antagonists, that inhibits at least BMP9. In some embodiments, a small molecule antagonist, or combination of small molecule antagonists, that inhibits BMP9 further inhibits one or more ligand [e.g., BMP10, BMP6, BMP5 and BMP3b], BMPRII, ALK1, endoglin, and/or one or more Smads. In some embodiments, a BMP antagonist is a small molecule antagonist, or combination of small molecule antagonists, that inhibits at least BMP6. In some embodiments, a small molecule antagonist, or combination of small molecule antagonists, that inhibits BMP6 further inhibits one or more ligand [e.g., BMP10, BMP9, BMP5 and BMP3b], BMPRII, ALK1, endoglin, and/or one or more Smads. In some embodiments, a BMP antagonist is a small molecule antagonist, or combination of small molecule antagonists, that inhibits at least BMP5. In some embodiments, a small molecule antagonist, or combination of small molecule antagonists, that inhibits BMP5 further inhibits one or more ligand [e.g., BMP10, BMP9, BMP5 and BMP3b], BMPRII, ALK1, endoglin, and/or one or more Smads. In some embodiments, a BMP antagonist is a small molecule antagonist, or combination of small molecule antagonists, that inhibits at least BMP3b. In some embodiments, a small molecule antagonist, or combination of small molecule antagonists, that inhibits BMP3b further inhibits one or more ligand [e.g., BMP10, BMP9, BMP6, and BMP5], BMPRII, ALK1, endoglin, and/or one or more Smads. In some embodiments, a BMP antagonist is a small molecule antagonist, or combination of small molecule antagonists, that inhibits at least BMPRII. In some embodiments, a small molecule antagonist, or combination of small molecule antagonists, that inhibits BMPRII further inhibits one or more ligand [e.g., BMP10, BMP9, BMP6, BMP5 and BMP3b], ALK1, endoglin, and/or one or more Smads. In some embodiments, a BMP antagonist is a small molecule antagonist, or combination of small molecule antagonists, that inhibits at least ALK1. In some embodiments, a small molecule antagonist, or combination of small molecule antagonists, that inhibits ALK1 further inhibits one or more ligand [e.g., BMP10, BMP9, BMP6, BMP5 and BMP3b], BMPRII, endoglin, and/or one or more Smads. In some embodiments, a BMP antagonist is a small molecule antagonist, or combination of small molecule antagonists, that inhibits at least endoglin. In some embodiments, a small molecule antagonist, or combination of small molecule antagonists, that inhibits endoglin further inhibits one or more ligand [e.g., BMP10, BMP9, BMP6, BMP5 and BMP3b], BMPRII, ALK1, and/or one or more Smads. In some embodiments, a BMP antagonist is a small molecule antagonist, or combination of small molecule antagonists, that inhibits at least one or more Smads. In some embodiments, a small molecule antagonist, or combination of small molecule antagonists, that inhibits Smads further inhibits one or more ligand [e.g., BMP10, BMP9, BMP6, BMP5 and BMP3b], BMPRII, ALK1, and/or endoglin.

Small molecule antagonists can be direct or indirect inhibitors. For example, an indirect small molecule antagonist, or combination of small molecule antagonists, may inhibit the expression (e.g., transcription, translation, cellular secretion, or combinations thereof) of at least one or more ligands [e.g., BMP10, BMP9, BMP6, BMP5 and BMP3b], BMPRII and/or ALK1, one or more co-receptors (endoglin), and/or one or more downstream signaling components (e.g., Smads). Alternatively, a direct small molecule BMP antagonist, or combination of small molecule antagonists, may directly bind to, for example, one or more of one or more ligands [e.g., BMP10, BMP9, BMP6, BMP5 and BMP3b], BMPRII and/or ALK1, one or more co-receptors (endoglin), and/or one or more downstream signaling components (e.g., Smads). Combinations of one or more indirect and one or more direct small molecule antagonists may be used in accordance with the methods disclosed herein.

Binding organic small molecule antagonists of the present disclosure may be identified and chemically synthesized using known methodology (see, e.g., PCT Publication Nos. WO 00/00823 and WO 00/39585). In general, small molecule antagonists of the disclosure are usually less than about 2000 daltons in size, alternatively less than about 1500, 750, 500, 250 or 200 daltons in size, wherein such organic small molecules that are capable of binding, preferably specifically, to a polypeptide as described herein. Such small molecule antagonists may be identified without undue experimentation using well-known techniques. In this regard, it is noted that techniques for screening organic small molecule libraries for molecules that are capable of binding to a polypeptide target are well-known in the art (see, e.g., international patent publication Nos. WO00/00823 and WO00/39585).

Binding organic small molecules of the present disclosure may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, and acid chlorides.

### 7. Polynucleotide Antagonists

In other aspects, the present disclosure relates to a BMP antagonist (inhibitor) that is a polynucleotide, or combination of polynucleotides. BMP antagonist polynucleotides may inhibit one or more ligands [e.g., BMP10, BMP9, BMP6, BMP5, and BMP3b], one or more of BMPRII, ALK1, and endoglin, and/or one or more downstream signaling components (e.g., Smads). In particular, the disclosure provides methods of using a BMP antagonist polynucleotide, or combination of BMP antagonist polynucleotides, alone or in combination with one or more additional supportive therapies and/or active agents, to treat, prevent, or reduce the progression rate and/or severity of pulmonary hypertension (PH), particularly treating, preventing or reducing the progression rate and/or severity of one or more PH-associated complications.

In some embodiments, a BMP antagonist is a polynucleotide antagonist, or combination of polynucleotide antagonists, that inhibits at least BMP10. In some embodiments, a polynucleotide antagonist, or combination of polynucleotide antagonists, that inhibits BMP10 further inhibits one or more ligand [e.g., BMP9, BMP6, BMP5, and BMP3b], one or more of BMPRII, ALK1, and endoglin, and/or one or more downstream signaling components (e.g., Smads). In some embodiments, a BMP antagonist is a polynucleotide antagonist, or combination of polynucleotide antagonists, that inhibits at least BMP9. In some embodiments, a polynucleotide antagonist, or combination of polynucleotide antagonists, that inhibits BMP9 further inhibits one or more ligand [e.g., BMP10, BMP6, BMP5, and BMP3b], one or more of BMPRII, ALK1, and endoglin, and/or one or more downstream signaling components (e.g., Smads). In some embodiments, a BMP antagonist is a polynucleotide antagonist, or combination of polynucleotide antagonists, that inhibits at least BMP6. In some embodiments, a polynucleotide antagonist, or combination of polynucleotide antagonists, that inhibits BMP6 further inhibits one or more ligand [e.g., BMP10, BMP9, BMP5, and BMP3b], one or more of BMPRII, ALK1, and endoglin, and/or one or more downstream signaling components (e.g., Smads). In some embodiments, a BMP antagonist is a polynucleotide antagonist, or combination of polynucleotide antagonists, that inhibits at least BMP5. In some embodiments, a polynucleotide antagonist, or combination of polynucleotide antagonists, that inhibits BMP5 further inhibits one or more ligand [e.g., BMP10, BMP9, BMP6, and BMP3b], one or more of BMPRII, ALK1, and endoglin, and/or one or more downstream signaling components (e.g., Smads). In some embodiments, a BMP antagonist is a polynucleotide antagonist, or combination of polynucleotide antagonists, that inhibits at least BMP3b. In some embodiments, a polynucleotide antagonist, or combination of polynucleotide antagonists, that inhibits BMP3b further inhibits one or more ligand [e.g., BMP10, BMP9, BMP6, and BMP5], one or more of BMPRII, ALK1, and endoglin, and/or one or more downstream signaling components (e.g., Smads). In some embodiments, a BMP antagonist is a polynucleotide antagonist, or combination of polynucleotide antagonists, that inhibits at least BMPRII. In some embodiments, a polynucleotide antagonist, or combination of polynucleotide antagonists, that inhibits BMPRII further inhibits one or more ligand [e.g., BMP10, BMP9, BMP6, BMP5, and BMP3b], ALK1 and/or endoglin, and/or one or more downstream signaling components (e.g., Smads). In some embodiments, a BMP antagonist is a polynucleotide antagonist, or combination of polynucleotide antagonists, that inhibits at least ALK1. In some embodiments, a polynucleotide antagonist, or combination of polynucleotide antagonists, that inhibits ALK1 further inhibits one or more ligands [e.g., BMP10, BMP9, BMP6, BMP5, and BMP3b], BMPRII and/or endoglin, and/or one or more downstream signaling components (e.g., Smads). In some embodiments, a BMP antagonist is a polynucleotide antagonist, or combination of polynucleotide antagonists, that inhibits at least endoglin. In some embodiments, a polynucleotide antagonist, or combination of polynucleotide antagonists, that inhibits endoglin further inhibits one or more ligands [e.g., BMP10, BMP9, BMP6, BMP5, and BMP3b], BMPRII and/or ALK1, and/or one or more downstream signaling components (e.g., Smads). In some embodiments, a BMP antagonist is a polynucleotide antagonist, or combination of polynucleotide antagonists, that inhibits at least one or more Smads. In some embodiments, a polynucleotide antagonist, or combination of polynucleotide antagonists, that inhibits one or more Smads further inhibits one or more ligands [e.g., BMP10, BMP9, BMP6, BMP5, and BMP3b] and/or one or more of BMPRII, ALK1, and endoglin.

In some embodiments, the polynucleotide antagonists of the present disclosure may be an antisense nucleic acid, an RNAi molecule [e.g., small interfering RNA (siRNA), small-hairpin RNA (shRNA), microRNA (miRNA)], an aptamer and/or a ribozyme. The nucleic acid and amino acid sequences of human BMP10, BMP9, BMP6, BMP5, BMP3b, BMPRII, ALK1, endoglin, and Smads are known in the art and thus polynucleotide antagonists for use in accordance with methods of the present disclosure may be routinely made by the skilled artisan based on the knowledge in the art and teachings provided herein.

For example, antisense technology can be used to control gene expression through antisense DNA or RNA, or through triple-helix formation. Antisense techniques are discussed, for example, in Okano (1991) J. Neurochem. 56:560; Oligodeoxynucleotides as Antisense Inhibitors of Gene Expression, CRC Press, Boca Raton, Fla. (1988). Triple helix formation is discussed in, for instance, Cooney et al. (1988) Science 241:456; and Dervan et al., (1991) Science 251: 1300. The methods are based on binding of a polynucleotide to a complementary DNA or RNA. In some embodiments, the antisense nucleic acids comprise a single-stranded RNA or DNA sequence that is complementary to at least a portion of an RNA transcript of a desired gene. However, absolute complementarity, although preferred, is not required.

A sequence “complementary to at least a portion of an RNA,” referred to herein, means a sequence having sufficient complementarity to be able to hybridize with the RNA, forming a stable duplex; in the case of double-stranded antisense nucleic acids of a gene disclosed herein, a single strand of the duplex DNA may thus be tested, or triplex formation may be assayed. The ability to hybridize will depend on both the degree of complementarity and the length of the antisense nucleic acid. Generally, the larger the hybridizing nucleic acid, the more base mismatches with an RNA it may contain and still form a stable duplex (or triplex as the case may be). One skilled in the art can ascertain a tolerable degree of mismatch by use of standard procedures to determine the melting point of the hybridized complex.

Polynucleotides that are complementary to the 5′ end of the message, for example, the 5′-untranslated sequence up to and including the AUG initiation codon, should work most efficiently at inhibiting translation. However, sequences complementary to the 3′-untranslated sequences of mRNAs have been shown to be effective at inhibiting translation of mRNAs as well [see, e.g., Wagner, R., (1994) Nature 372:333-335]. Thus, oligonucleotides complementary to either the 5′- or 3′-untranslated, noncoding regions of a gene of the disclosure, could be used in an antisense approach to inhibit translation of an endogenous mRNA. Polynucleotides complementary to the 5′-untranslated region of the mRNA should include the complement of the AUG start codon. Antisense polynucleotides complementary to mRNA coding regions are less efficient inhibitors of translation but could be used in accordance with the methods of the present disclosure. Whether designed to hybridize to the 5′-untranslated, 3′-untranslated, or coding regions of an mRNA of the disclosure, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides, or at least 50 nucleotides.

In one embodiment, the antisense nucleic acid of the present disclosure is produced intracellularly by transcription from an exogenous sequence. For example, a vector or a portion thereof, is transcribed, producing an antisense nucleic acid (RNA) of a gene of the disclosure. Such a vector would contain a sequence encoding the desired antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in vertebrate cells. Expression of the sequence encoding desired genes of the instant disclosure, or fragments thereof, can be by any promoter known in the art to act in vertebrate, preferably human cells. Such promoters can be inducible or constitutive. Such promoters include, but are not limited to, the SV40 early promoter region [see, e.g., Benoist and Chambon (1981) Nature 29:304-310], the promoter contained in the 3′ long terminal repeat of Rous sarcoma virus [see, e.g., Yamamoto et al. (1980) Cell 22:787-797], the herpes thymidine promoter [see, e.g., Wagner et al. (1981) Proc. Natl. Acad. Sci. U.S.A. 78: 1441-1445], and the regulatory sequences of the metallothionein gene [see, e.g., Brinster, et al. (1982) Nature 296:39-42].

In some embodiments, the polynucleotide antagonists are interfering RNA or RNAi molecules that target the expression of one or more genes. RNAi refers to the expression of an RNA which interferes with the expression of the targeted mRNA. Specifically, RNAi silences a targeted gene via interacting with the specific mRNA through a siRNA (small interfering RNA). The ds RNA complex is then targeted for degradation by the cell. An siRNA molecule is a double-stranded RNA duplex of 10 to 50 nucleotides in length, which interferes with the expression of a target gene which is sufficiently complementary (e.g. at least 80% identity to the gene). In some embodiments, the siRNA molecule comprises a nucleotide sequence that is at least 85, 90, 95, 96, 97, 98, 99, or 100% identical to the nucleotide sequence of the target gene.

Additional RNAi molecules include short-hairpin RNA (shRNA); also short-interfering hairpin and microRNA (miRNA). The shRNA molecule contains sense and antisense sequences from a target gene connected by a loop. The shRNA is transported from the nucleus into the cytoplasm, and it is degraded along with the mRNA. Pol III or U6 promoters can be used to express RNAs for RNAi. Paddison et al. [Genes & Dev. (2002) 16:948-958, 2002] have used small RNA molecules folded into hairpins as a means to effect RNAi. Accordingly, such short hairpin RNA (shRNA) molecules are also advantageously used in the methods described herein. The length of the stem and loop of functional shRNAs varies; stem lengths can range anywhere from about 25 to about 30 nt, and loop size can range between 4 to about 25 nt without affecting silencing activity. While not wishing to be bound by any particular theory, it is believed that these shRNAs resemble the double-stranded RNA (dsRNA) products of the DICER RNase and, in any event, have the same capacity for inhibiting expression of a specific gene. The shRNA can be expressed from a lentiviral vector. An miRNA is a single-stranded RNA of about 10 to 70 nucleotides in length that are initially transcribed as pre-miRNA characterized by a “stem-loop” structure and which are subsequently processed into mature miRNA after further processing through the RISC.

Molecules that mediate RNAi, including without limitation siRNA, can be produced in vitro by chemical synthesis (Hohjoh, FEBS Lett 521:195-199, 2002), hydrolysis of dsRNA (Yang et al., Proc Natl Acad Sci USA 99:9942-9947, 2002), by in vitro transcription with T7 RNA polymerase (Donzeet et al., Nucleic Acids Res 30:e46, 2002; Yu et al., Proc Natl Acad Sci USA 99:6047-6052, 2002), and by hydrolysis of double-stranded RNA using a nuclease such as E. coli RNase III (Yang et al., Proc Natl Acad Sci USA 99:9942-9947, 2002).

According to another aspect, the disclosure provides polynucleotide antagonists including but not limited to, a decoy DNA, a double-stranded DNA, a single-stranded DNA, a complexed DNA, an encapsulated DNA, a viral DNA, a plasmid DNA, a naked RNA, an encapsulated RNA, a viral RNA, a double-stranded RNA, a molecule capable of generating RNA interference, or combinations thereof.

In some embodiments, the polynucleotide antagonists of the disclosure are aptamers. Aptamers are nucleic acid molecules, including double-stranded DNA and single-stranded RNA molecules, which bind to and form tertiary structures that specifically bind to a target molecule, such as a BMP10, BMP9, BMP6, BMP5, BMP3b, BMPRII, ALK1, endoglin, and Smads. The generation and therapeutic use of aptamers are well established in the art. See, e.g., U.S. Pat. No. 5,475,096. Additional information on aptamers can be found in U.S. Pat. Application Publication No. 20060148748. Nucleic acid aptamers are selected using methods known in the art, for example via the Systematic Evolution of Ligands by Exponential Enrichment (SELEX) process. SELEX is a method for the in vitro evolution of nucleic acid molecules with highly specific binding to target molecules as described in, e.g., U.S. Pat. Nos. 5,475,096, 5,580,737, 5,567,588, 5,707,796, 5,763,177, 6,011,577, and 6,699,843. Another screening method to identify aptamers is described in U.S. Pat. No. 5,270,163. The SELEX process is based on the capacity of nucleic acids for forming a variety of two- and three-dimensional structures, as well as the chemical versatility available within the nucleotide monomers to act as ligands (form specific binding pairs) with virtually any chemical compound, whether monomeric or polymeric, including other nucleic acid molecules and polypeptides. Molecules of any size or composition can serve as targets. The SELEX method involves selection from a mixture of candidate oligonucleotides and stepwise iterations of binding, partitioning and amplification, using the same general selection scheme, to achieve desired binding affinity and selectivity. Starting from a mixture of nucleic acids, which can comprise a segment of randomized sequence, the SELEX method includes steps of contacting the mixture with the target under conditions favorable for binding; partitioning unbound nucleic acids from those nucleic acids which have bound specifically to target molecules; dissociating the nucleic acid-target complexes; amplifying the nucleic acids dissociated from the nucleic acid-target complexes to yield a ligand enriched mixture of nucleic acids. The steps of binding, partitioning, dissociating and amplifying are repeated through as many cycles as desired to yield highly specific high affinity nucleic acid ligands to the target molecule.

Typically, such binding molecules are separately administered to the animal [see, e.g., O’Connor (1991) J. Neurochem. 56:560], but such binding molecules can also be expressed in vivo from polynucleotides taken up by a host cell and expressed in vivo [see, e.g., Oligodeoxynucleotides as Antisense Inhibitors of Gene Expression, CRC Press, Boca Raton, Fla. (1988)].

### 8. Screening Assays

In certain aspects, the present disclosure relates to the use of the subject BMP antagonists (e.g., BMP10pro polypeptides and variants thereof) to identify compounds (agents) which may be used to treat, prevent, or reduce the progression rate and/or severity of pulmonary hypertension (PH), particularly treating, preventing or reducing the progression rate and/or severity of one or more PH-associated complications. These compounds can be tested, for example, in animal models.

There are numerous approaches to screening for therapeutic agents for treating PH (e.g., pulmonary arterial hypertension) by targeting TGFβ superfamily ligand signaling (e.g., SMAD signaling). In certain embodiments, high-throughput screening of compounds can be carried out to identify agents that perturb TGFβ superfamily receptor-mediated effects on a selected cell line. In certain embodiments, the assay is carried out to screen and identify compounds that specifically inhibit or reduce binding of a BMP 10 propeptides to a binding partner including for example, BMP10, BMP9, BMP6, BMP5, and BMP3b. Alternatively, the assay can be used to identify compounds that enhance binding of a BMP10 propeptides to a binding partner such as a ligand. In a further embodiment, the compounds can be identified by their ability to interact with a BMP10 propeptides.

A variety of assay formats will suffice and, in light of the present disclosure, those not expressly described herein will nevertheless be comprehended by one of ordinary skill in the art. As described herein, the test compounds (agents) of the invention may be created by any combinatorial chemical method. Alternatively, the subject compounds may be naturally occurring biomolecules synthesized in vivo or in vitro. Compounds (agents) to be tested for their ability to act as modulators of PH can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In certain embodiments, the test agent is a small organic molecule having a molecular weight of less than about 2,000 Daltons.

The test compounds of the disclosure can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S-transferase (GST), photoactivatible crosslinkers or any combinations thereof.

In many drug-screening programs which test libraries of compounds and natural extracts, high-throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between a BMP10 propeptides to a binding partner including for example, BMP10, BMP9, BMP6, BMP5, and BMP3b.

Merely to illustrate, in an exemplary screening assay of the present disclosure, the compound of interest is contacted with an isolated and purified BMP10 propeptide which is ordinarily capable of binding to a TGF-beta superfamily ligand, as appropriate for the intention of the assay. To the mixture of the compound and BMP10 propeptide is then added to a composition containing the appropriate ligand (e.g., BMP10, BMP9, BMP6, BMP5, and BMP3b). Detection and quantification of BMP10 propeptide-superfamily ligand complexes provides a means for determining the compound’s efficacy at inhibiting (or potentiating) complex formation between the BMP10 propeptide and its binding protein. The efficacy of the compound can be assessed by generating dose-response curves from data obtained using various concentrations of the test compound. Moreover, a control assay can also be performed to provide a baseline for comparison. For example, in a control assay, isolated and purified ligand is added to a composition containing the BMP10 propeptide, and the formation of BMP10 propeptide-ligand complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Binding of a BMP10 propeptide to another protein may be detected by a variety of techniques. For instance, modulation of the formation of complexes can be quantitated using, for example, detectably labeled proteins such as radiolabeled (e.g., ³²P, ³⁵S, ¹⁴C or ³H), fluorescently labeled (e.g., FITC), or enzymatically labeled BMP10 propeptide and/or a binding protein, by immunoassay, or by chromatographic detection.

In certain embodiments, the present disclosure contemplates the use of fluorescence polarization assays and fluorescence resonance energy transfer (FRET) assays in measuring, either directly or indirectly, the degree of interaction between a BMP10 propeptide and a binding protein. Further, other modes of detection, such as those based on optical waveguides (See, e.g., PCT Publication WO 96/26432 and U.S. Pat. No. 5,677,196), surface plasmon resonance (SPR), surface charge sensors, and surface force sensors, are compatible with many embodiments of the disclosure.

Moreover, the present disclosure contemplates the use of an interaction trap assay, also known as the “two-hybrid assay,” for identifying agents that disrupt or potentiate interaction between a BMP10 propeptide and a binding partner. See, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present disclosure contemplates the use of reverse two-hybrid systems to identify compounds (e.g., small molecules or peptides) that dissociate interactions between a BMP10 propeptide and a binding protein [Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368].

In certain embodiments, the subject compounds are identified by their ability to interact with a BMP10 propeptide. The interaction between the compound and the BMP10 propeptide may be covalent or non-covalent. For example, such interaction can be identified at the protein level using in vitro biochemical methods, including photo-crosslinking, radiolabeled ligand binding, and affinity chromatography [Jakoby WB et al. (1974) Methods in Enzymology 46:1]. In certain cases, the compounds may be screened in a mechanism-based assay, such as an assay to detect compounds which bind to a BMP10 propeptide. This may include a solid-phase or fluid-phase binding event. Alternatively, the gene encoding a BMP10 propeptide can be transfected with a reporter system (e.g., β-galactosidase, luciferase, or green fluorescent protein) into a cell and screened against the library preferably by high-throughput screening or with individual members of the library. Other mechanism-based binding assays may be used; for example, binding assays which detect changes in free energy. Binding assays can be performed with the target fixed to a well, bead or chip or captured by an immobilized antibody or resolved by capillary electrophoresis. The bound compounds may be detected usually using colorimetric endpoints or fluorescence or surface plasmon resonance.

### 9. Therapeutic Uses

In part, the present disclosure relates to methods of treating pulmonary hypertension (e.g., pulmonary arterial hypertension) comprising administering to a patient in need thereof an effective amount of any of the subject BMP antagonists disclosed herein (e.g., BMP10pro polypeptides and variants thereof). In some embodiments, the disclosure contemplates methods of treating one or more complications of pulmonary hypertension (e.g., smooth muscle and/or endothelial cell proliferation in the pulmonary artery, angiogenesis in the pulmonary artery, dyspnea, chest pain, pulmonary vascular remodeling, right ventricular hypertrophy, and pulmonary fibrosis) comprising administering to a patient in need thereof an effective amount of a BMP antagonist. In some embodiments, the disclosure contemplates methods of preventing one or more complications of pulmonary hypertension comprising administering to a patient in need thereof an effective amount of a BMP antagonist. In some embodiments, the disclosure contemplates methods of reducing the progression rate of pulmonary hypertension comprising administering to a patient in need thereof an effective amount of a BMP antagonist. In some embodiments, the disclosure contemplates methods of reducing the progression rate of one or more complications of pulmonary hypertension comprising administering to a patient in need thereof an effective amount of a BMP antagonist. In some embodiments, the disclosure contemplates methods of reducing the severity of pulmonary hypertension comprising administering to a patient in need thereof an effective amount of a BMP antagonist. In some embodiments, the disclosure contemplates methods of reducing the severity of one or more complications of pulmonary hypertension comprising administering to a patient in need thereof an effective amount of a BMP antagonist. Optionally, methods disclosed herein for treating, preventing, or reducing the progression rate and/or severity of pulmonary hypertension, particularly treating, preventing, or reducing the progression rate and/or severity of one or more complications of pulmonary hypertension, may further comprise administering to the patient one or more supportive therapies or additional active agents for treating pulmonary hypertension. For example, the patient also may be administered one or more supportive therapies or active agents selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-_{K}B antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1-->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1-->3)-beta-D-glucopyranosyl] oleanolic acid 28-O-beta-D-glucopyranosyl ester; 3-O-[beta-D-glucopyranosyl-(1-->2)-beta-D-glucopyranosyl] oleanolic acid 28-O-beta-D-glucopyranosyl ester; 3-O-[a-L-rhamnopyranosyl-(1-->3)-beta-D-glucuronopyranosyl] oleanolic acid; 3-O-[alpha-L-rhamnopyranosyl-(1-->3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1—>3)-β-D-glucopyranosiduronic acid (CS1); oleanolic acid 3-O-β-D-glucopyranosyl (1—>3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3ʹ,4ʹ:2,3]oleanolate) lung and/or heart transplantation. In some embodiments, the patient may also be administered a BMP9 prodomain polypeptide. In some embodiments, the BMP9 prodomain polypeptide is administered in a pharmaceutical preparation. BMP9 prodomain polypeptides, pharmaceutical compositions comprising BMP9 prodomain polypeptides, and methods of generating such polypeptides and pharmaceutical compositions are described in, for example, WO 2013/152213, which is incorporated by reference herein in its entirety. In some embodiments, the patient may also be administered an ActRIIA polypeptide. In some embodiments, the ActRIIA polypeptide is a fusion protein comprising an ActRIIA domain and one or more polypeptide domains heterologous to ActRIIA. In some embodiments, the ActRIIA polypeptide is administered in a pharmaceutical preparation. ActRIIA polypeptides, pharmaceutical compositions comprising ActRIIA polypeptides, and methods of generating such polypeptides and pharmaceutical compositions are described in, for example, WO 2018/013936, which is incorporated by reference herein in its entirety.As used herein, a therapeutic that “prevents” a disorder or condition refers to a compound that, in a statistical sample, reduces the occurrence of the disorder or condition in the treated sample relative to an untreated control sample, or delays the onset one or more symptoms of the disorder or condition relative to the untreated control sample.

In some embodiments, the present disclosure relates to methods of treating an interstitial lung disease (e.g., idiopathic pulmonary fibrosis) comprising administering to a patient in need thereof an effective amount of any of the BMP antagonists disclosed herein (e.g., an antagonist of one or more of BMP10, BMP9, BMP6, BMP5, and BMP3b). In some embodiments, the interstitial lung disease is pulmonary fibrosis. In some embodiments, the interstitial lung disease is caused by any one of the following: silicosis, asbestosis, berylliosis, hypersensitivity pneumonitis, drug use (e.g., antibiotics, chemotherapeutic drugs, antiarrhythmic agents, statins), systemic sclerosis, polymyositis, dermatomyositis, systemic lupus erythematosus, rheumatoid arthritis, an infection (e.g., atypical pneumonia, pneumocystis pneumonia, tuberculosis, chlamydia trachomatis, and/or respiratory syncytial virus), lymphangitic carcinomatosis, cigarette smoking, or developmental disorders. In some embodiments, the interstitial lung disease is idiopathic (e.g., sarcoidosis, idiopathic pulmonary fibrosis, Hamman-Rich syndrome, and/or antisynthetase syndrome). In particular embodiments, the interstitial lung disease is idiopathic pulmonary fibrosis. In some embodiments, the treatment for idiopathic pulmonary fibrosis is administered in combination with an additional therapeutic agent. In some embodiments, the additional therapeutic agent is selected from the group consisting of: pirfenidone, N-acetylcysteine, prednisone, azathioprine, nintedanib, derivatives thereof and combinations thereof.

The term “treating” as used herein includes amelioration or elimination of the condition once it has been established. In either case, prevention or treatment may be discerned in the diagnosis provided by a physician or other health care provider and the intended result of administration of the therapeutic agent.

As used herein, “in combination with”, “combinations of”, “combined with”, “conjoint” administration and the like refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more BMP10 antagonists of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the BMP10 antagonist of the present disclosure with the additional active agent or therapy and/or the desired effect.

In general, treatment or prevention of a disease or condition as described in the present disclosure is achieved by administering a BMP antagonist in an effective amount. An effective amount of an agent refers to an amount effective, at dosages and for periods of time necessary, to achieve the desired therapeutic or prophylactic result. A therapeutically effective amount of an agent of the present disclosure may vary according to factors such as the disease state, age, sex, and weight of the individual, and the ability of the agent to elicit a desired response in the individual. A prophylactically effective amount refers to an amount effective, at dosages and for periods of time necessary, to achieve the desired prophylactic result.

The terms “subject,” an “individual,” or a “patient” are interchangeable throughout the specification and generally refer to mammals. Mammals include, but are not limited to, domesticated animals (e.g., cows, sheep, cats, dogs, and horses), primates (e.g., humans and non-human primates such as monkeys), rabbits, and rodents (e.g., mice and rats).

Pulmonary hypertension (PH) has been previously classified as primary (idiopathic) or secondary. Recently, the World Health Organization (WHO) has classified pulmonary hypertension into five groups: Group 1: pulmonary arterial hypertension (PAH); Group 2: pulmonary hypertension with left heart disease; Group 3: pulmonary hypertension with lung disease and/or hypoxemia; Group 4: pulmonary hypertension due to chronic thrombotic and/or embolic disease; and Group 5: miscellaneous conditions (e.g., sarcoidosis, histiocytosis X, lymphangiomatosis and compression of pulmonary vessels). See, for example, Rubin (2004) Chest 126:7-10.

In certain aspects, the disclosure relates to methods of treating, preventing, or reducing the progression rate and/or severity of pulmonary hypertension (e.g., treating, preventing, or reducing the progression rate and/or severity of one or more complications of pulmonary hypertension) comprising administering to a patient in need thereof an effective amount of a BMP antagonist (e.g., an antagonist of one or more of BMP 10, BMP9, BMP6, BMP5, and BMP3b). In some embodiments, the method relates to pulmonary hypertension patients that have pulmonary arterial hypertension. In some embodiments, the method relates pulmonary hypertension patients that have pulmonary hypertension with left heart disease. In some embodiments, the method relates to pulmonary hypertension patients that have lung disease and/or hypoxemia. In some embodiments, the method relates to pulmonary hypertension patients that have chronic thrombotic and/or embolic disease. In some embodiments, the method relates to pulmonary hypertension patients that have sarcoidosis, histiocytosis X, or lymphangiomatosis and compression of pulmonary vessels.

Pulmonary arterial hypertension is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary arterial pressures. These high pressures make it difficult for the heart to pump blood through the lungs to be oxygenated. Patients with PAH suffer from extreme shortness of breath as the heart struggles to pump against these high pressures. Patients with PAH typically develop significant increases in pulmonary vascular resistance (PVR) and sustained elevations in pulmonary artery pressure (PAP), which ultimately lead to right ventricular failure and death. Patients diagnosed with PAH have a poor prognosis and equally compromised quality of life, with a mean life expectancy of 2 to 5 years from the time of diagnosis if untreated.

A variety of factors contribute to the pathogenesis of pulmonary hypertension including proliferation of pulmonary cells which can contribute to vascular remodeling (i.e., hyperplasia). For example, pulmonary vascular remodeling occurs primarily by proliferation of arterial endothelial cells and smooth muscle cells of patients with pulmonary hypertension. Overexpression of various cytokines is believed to promote pulmonary hypertension. Further, it has been found that pulmonary hypertension may rise from the hyperproliferation of pulmonary arterial smooth cells and pulmonary endothelial cells. Still further, advanced PAH may be characterized by muscularization of distal pulmonary arterioles, concentric intimal thickening, and obstruction of the vascular lumen by proliferating endothelial cells. Pietra et al., J. Am. Coll. Cardiol., 43:255-325 (2004).

In certain aspects, the disclosure relates to methods of treating, preventing, or reducing the progression rate and/or severity of pulmonary hypertension (e.g., treating, preventing, or reducing the progression rate and/or severity of one or more complications of pulmonary hypertension) comprising administering to a patient in need thereof an effective amount of a BMP antagonist (e.g., an antagonist of one or more of BMP 10, BMP9, BMP6, BMP5, and BMP3b), wherein the patient has resting pulmonary arterial pressure (PAP) of at least 25 mm Hg (e.g., 25, 30, 35, 40, 45, or 50 mm Hg). In some embodiments, the method relates to patients having a resting PAP of at least 25 mm Hg. In some embodiments, the method relates to patients having a resting PAP of at least 30 mm Hg. In some embodiments, the method relates to patients having a resting PAP of at least 35 mm Hg. In some embodiments, the method relates to patients having a resting PAP of at least 40 mm Hg. In some embodiments, the method relates to patients having a resting PAP of at least 45 mm Hg. In some embodiments, the method relates to patients having a resting PAP of at least 50 mm Hg.

In some embodiments, the disclosure relates to methods of adjusting one or more hemodynamic parameters in the PH patient toward a more normal level (e.g., normal as compared to healthy people of similar age and sex), comprising administering to a patient in need thereof an effective amount of a BMP antagonist (e.g., an antagonist of one or more of BMP10, BMP9, BMP6, BMP5, and BMP3b). In some embodiments, the method relates to reducing PAP. In some embodiments, the method relates to reducing the patient’s PAP by at least 3 mmHg. In certain embodiments, the method relates to reducing the patient’s PAP by at least 5 mmHg. In certain embodiments, the method relates to reducing the patient’s PAP by at least 7 mmHg. In certain embodiments, the method relates to reducing the patient’s PAP by at least 10 mmHg. In certain embodiments, the method relates to reducing the patient’s PAP by at least 12 mmHg. In certain embodiments, the method relates to reducing the patient’s PAP by at least 15 mmHg. In certain embodiments, the method relates to reducing the patient’s PAP by at least 20 mmHg. In certain embodiments, the method relates to reducing the patient’s PAP by at least 25 mmHg. In some embodiments, the method relates to reducing pulmonary vascular resistance (PVR). In some embodiments, the method relate to increasing pulmonary capillary wedge pressure (PCWP). In some embodiments, the method relate to increasing left ventricular end-diastolic pressure (LVEDP).

In certain aspects, the disclosure relates to methods of treating, preventing, or reducing the progression rate and/or severity of one or more complications of pulmonary hypertension comprising administering to a patient in need thereof an effective amount of a BMP antagonist (e.g., an antagonist of one or more of BMP 10, BMP9, BMP6, BMP5, and BMP3b). In some embodiments, the method relates to treating, preventing, or reducing the progression rate and/or severity of cell proliferation in the pulmonary artery of a pulmonary hypertension patient. In some embodiments, the method relates to treating, preventing, or reducing the progression rate and/or severity of smooth muscle and/or endothelial cells proliferation in the pulmonary artery of a pulmonary hypertension patient. In some embodiments, the method relates to treating, preventing, or reducing the progression rate and/or severity of angiogenesis in the pulmonary artery of a pulmonary hypertension patient. In some embodiments, the method relates to increasing physical activity of a patient having pulmonary hypertension. In some embodiments, the method relates to treating, preventing, or reducing the progression rate and/or severity of dyspnea in a pulmonary hypertension patient. In some embodiments, the method relates to treating, preventing, or reducing the progression rate and/or severity of chest pain in a pulmonary hypertension patient. In some embodiments, the method relates to treating, preventing, or reducing the progression rate and/or severity of fatigue in a pulmonary hypertension patient. In some embodiments, the method relates to treating, preventing, or reducing the progression rate and/or severity of pulmonary fibrosis in a pulmonary hypertension patient. In some embodiments, the method relates to treating, preventing, or reducing the progression rate and/or severity of fibrosis in a pulmonary hypertension patient. In some embodiments, the method relates to treating, preventing, or reducing the progression rate and/or severity of pulmonary vascular remodeling in a pulmonary hypertension patient. In some embodiments, the method relates to treating, preventing, or reducing the progression rate and/or severity of right ventricular hypertrophy in a pulmonary hypertension patient.

In certain aspects, the disclosure relates to methods of increasing exercise capacity in a patient having pulmonary hypertension comprising administering to a patient in need thereof an effective amount of a BMP antagonist (e.g., an antagonist of one or more of BMP10, BMP9, BMP6, BMP5, and BMP3b). Any suitable measure of exercise capacity can be used. For example, exercise capacity in a 6-minute walk test (6 MWT), which measures how far the subject can walk in 6 minutes, i.e., the 6-minute walk distance (6 MWD), is frequently used to assess pulmonary hypertension severity and disease progression. The Borg dyspnea index (BDI) is a numerical scale for assessing perceived dyspnea (breathing discomfort). It measures the degree of breathlessness, for example, after completion of the 6 MWT, where a BDI of 0 indicates no breathlessness and 10 indicates maximum breathlessness. In some embodiments, the method relates to increasing 6 MWD by at least 10 meters in the patient having pulmonary hypertension. In some embodiments, the method relates to increasing 6 MWD by at least 20 meters in the patient having pulmonary hypertension. In some embodiments, the method relates to increasing 6 MWD by at least 30 meters in the patient having pulmonary hypertension. In some embodiments, the method relates to increasing 6 MWD by at least 40 meters in the patient having pulmonary hypertension. In some embodiments, the method relates to increasing 6 MWD by at least 50 meters in the patient having pulmonary hypertension. In some embodiments, the method relates to increasing 6 MWD by at least 60 meters in the patient having pulmonary hypertension. In some embodiments, the method relates to increasing 6 MWD by at least 70 meters in the patient having pulmonary hypertension. In some embodiments, the method relates to increasing 6 MWD by at least 80 meters in the patient having pulmonary hypertension. In some embodiments, the method relates to increasing 6 MWD by at least 90 meters in the patient having pulmonary hypertension. In some embodiments, the method relates to increasing 6 MWD by at least 100 meters in the patient having pulmonary hypertension. In some embodiments, the method relate to lowering BDI by at least 0.5 index points in the patient having pulmonary hypertension. In some embodiments, the method relate to lowering BDI by at least 1 index points in the patient having pulmonary hypertension. In some embodiments, the method relate to lowering BDI by at least 1.5 index points in the patient having pulmonary hypertension. In some embodiments, the method relate to lowering BDI by at least 2 index points in the patient having pulmonary hypertension. In some embodiments, the method relate to lowering BDI by at least 2.5 index points in the patient having pulmonary hypertension. In some embodiments, the method relate to lowering BDI by at least 3 index points in the patient having pulmonary hypertension. In some embodiments, the method relate to lowering BDI by at least 3.5 index points in the patient having pulmonary hypertension. In some embodiments, the method relate to lowering BDI by at least 4 index points in the patient having pulmonary hypertension. In some embodiments, the method relate to lowering BDI by at least 4.5 index points in the patient having pulmonary hypertension. In some embodiments, the method relate to lowering BDI by at least 5 index points in the patient having pulmonary hypertension. In some embodiments, the method relate to lowering BDI by at least 5.5 index points in the patient having pulmonary hypertension. In some embodiments, the method relate to lowering BDI by at least 6 index points in the patient having pulmonary hypertension. In some embodiments, the method relate to lowering BDI by at least 6.5 index points in the patient having pulmonary hypertension. In some embodiments, the method relate to lowering BDI by at least 7 index points in the patient having pulmonary hypertension. In some embodiments, the method relate to lowering BDI by at least 7.5 index points in the patient having pulmonary hypertension. In some embodiments, the method relate to lowering BDI by at least 8 index points in the patient having pulmonary hypertension. In some embodiments, the method relate to lowering BDI by at least 8.5 index points in the patient having pulmonary hypertension. In some embodiments, the method relate to lowering BDI by at least 9 index points in the patient having pulmonary hypertension. In some embodiments, the method relate to lowering BDI by at least 9.5 index points in the patient having pulmonary hypertension. In some embodiments, the method relate to lowering BDI by at least 3 index points in the patient having pulmonary hypertension. In some embodiments, the method relate to lowering BDI by 10 index points in the patient having pulmonary hypertension.

Pulmonary hypertension at baseline can be mild, moderate or severe, as measured for example by World Health Organization (WHO) functional class, which is a measure of disease severity in patients with pulmonary hypertension. The WHO functional classification is an adaptation of the New York Heart Association (NYHA) system and is routinely used to qualitatively assess activity tolerance, for example in monitoring disease progression and response to treatment (Rubin (2004) Chest 126:7-10). Four functional classes are recognized in the WHO system: Class I: pulmonary hypertension without resulting limitation of physical activity; ordinary physical activity does not cause undue dyspnea or fatigue, chest pain or near syncope; Class II: pulmonary hypertension resulting in slight limitation of physical activity; patient comfortable at rest; ordinary physical activity causes undue dyspnea or fatigue, chest pain or near syncope; Class III: pulmonary hypertension resulting in marked limitation of physical activity; patient comfortable at rest; less than ordinary activity causes undue dyspnea or fatigue, chest pain or near syncope; Class IV: pulmonary hypertension resulting in inability to carry out any physical activity without symptoms; patient manifests signs of right-heart failure; dyspnea and/or fatigue may be present even at rest; discomfort is increased by any physical activity.

In certain aspects, the disclosure relates to methods of treating, preventing, or reducing the progression rate and/or severity of pulmonary hypertension (e.g., treating, preventing, or reducing the progression rate and/or severity of one or more complications of pulmonary hypertension) comprising administering to a patient in need thereof an effective amount of a BMP antagonist (e.g., an antagonist of one or more of BMP 10, BMP9, BMP6, BMP5, and BMP3b), wherein the patient has Class I, Class II, Class III, or Class IV pulmonary hypertension as recognized by the WHO. In some embodiments, the method relates to a patient that has Class I pulmonary hypertension as recognized by the WHO. In some embodiments, the method relates to a patient that has Class II pulmonary hypertension as recognized by the WHO. In some embodiments, the method relates to preventing or delaying patient progression from Class I pulmonary hypertension to Class II pulmonary hypertension as recognized by the WHO. In some embodiments, the method relates to promoting or increasing patient regression from Class II pulmonary hypertension to Class I pulmonary hypertension as recognized by the WHO. In some embodiments, the method relates to a patient that has Class III pulmonary hypertension as recognized by the WHO. In some embodiments, the method relates to preventing or delaying patient progression from Class II pulmonary hypertension to Class III pulmonary hypertension as recognized by the WHO. In some embodiments, the method relates to promoting or increasing patient regression from Class III pulmonary hypertension to Class II pulmonary hypertension as recognized by the WHO. In some embodiments, the method relates to promoting or increasing patient regression from Class III pulmonary hypertension to Class I pulmonary hypertension as recognized by the WHO. In some embodiments, the method relates to a patient that has Class IV pulmonary hypertension as recognized by the WHO. In some embodiments, the method relates to preventing or delaying patient progression from Class III pulmonary hypertension to Class IV pulmonary hypertension as recognized by the WHO. In some embodiments, the method relates to promoting or increasing patient regression from Class IV pulmonary hypertension to Class III pulmonary hypertension as recognized by the WHO. In some embodiments, the method relates to promoting or increasing patient regression from Class IV pulmonary hypertension to Class II pulmonary hypertension as recognized by the WHO. In some embodiments, the method relates to promoting or increasing patient regression from Class IV pulmonary hypertension to Class I pulmonary hypertension as recognized by the WHO.

There is no known cure for pulmonary hypertension; current methods of treatment focus on prolonging patient lifespan and enhancing patient quality of life. Current methods of treatment of pulmonary hypertension include administration of: vasodilators such as prostacyclin, epoprostenol, and sildenafil; endothelin receptor antagonists such as bosentan; calcium channel blockers such as amlodipine, diltiazem, and nifedipine; anticoagulants such as warfarin; and diuretics. Treatment of pulmonary hypertension has also been carried out using oxygen therapy, atrial septostomy, pulmonary thromboendarterectomy, and lung and/or heart transplantation. Each of these methods, however, suffers from one or multiple drawbacks which may include lack of effectiveness, serious side effects, low patient compliance, and high cost. In certain aspects, the method relate to treating, preventing, or reducing the progression rate and/or severity of pulmonary hypertension (e.g., treating, preventing, or reducing the progression rate and/or severity of one or more complications of pulmonary hypertension) comprising administering to a patient in need thereof an effective amount of a BMP antagonist (e.g., an antagonist of one or more of BMP 10, BMP9, BMP6, BMP5, and BMP3b) in combination (e.g., administered at the same time or different times, but generally in such a manner as to achieve overlapping pharmacological/physiological effects) with one or more additional active agents and/or supportive therapies for treating pulmonary hypertension (e.g., vasodilators such as prostacyclin, epoprostenol, and sildenafil; endothelin receptor antagonists such as bosentan; calcium channel blockers such as amlodipine, diltiazem, and nifedipine; anticoagulants such as warfarin; diuretics; oxygen therapy; atrial septostomy; pulmonary thromboendarterectomy: and lung and/or heart transplantation);; bardoxolone methyl or a derivative thereof; oleanolic acid or derivative thereof.

In certain embodiments, the present disclosure provides methods for managing a patient that has been treated with, or is a candidate to be treated with, one or more one or more BMP antagonists of the disclosure (e.g., antagonists that inhibit one or more BMP ligands (e.g., BMP10, BMP9, BMP6, BMP3b, and BMP5); antagonists that inhibit one or more of BMPRII, ALK1, and endoglin; and antagonists that inhibit one or more downstream signaling components (e.g., Smad proteins)) by measuring one or more hematologic parameters in the patient. The hematologic parameters may be used to evaluate appropriate dosing for a patient who is a candidate to be treated with the antagonist of the present disclosure, to monitor the hematologic parameters during treatment, to evaluate whether to adjust the dosage during treatment with one or more antagonist of the disclosure, and/or to evaluate an appropriate maintenance dose of one or more antagonists of the disclosure. If one or more of the hematologic parameters are outside the normal level, dosing with one or more BMP antagonists may be reduced, delayed or terminated.

Hematologic parameters that may be measured in accordance with the methods provided herein include, for example, red blood cell levels, blood pressure, iron stores, and other agents found in bodily fluids that correlate with increased red blood cell levels, using art recognized methods. Such parameters may be determined using a blood sample from a patient. Increases in red blood cell levels, hemoglobin levels, and/or hematocrit levels may cause increases in blood pressure.

In one embodiment, if one or more hematologic parameters are outside the normal range or on the high side of normal in a patient who is a candidate to be treated with one or more BMP antagonists, then onset of administration of the one or more antagonists of the disclosure may be delayed until the hematologic parameters have returned to a normal or acceptable level either naturally or via therapeutic intervention. For example, if a candidate patient is hypertensive or pre-hypertensive, then the patient may be treated with a blood pressure lowering agent in order to reduce the patient’s blood pressure. Any blood pressure lowering agent appropriate for the individual patient’s condition may be used including, for example, diuretics, adrenergic inhibitors (including alpha blockers and beta blockers), vasodilators, calcium channel blockers, angiotensin-converting enzyme (ACE) inhibitors, or angiotensin II receptor blockers. Blood pressure may alternatively be treated using a diet and exercise regimen. Similarly, if a candidate patient has iron stores that are lower than normal, or on the low side of normal, then the patient may be treated with an appropriate regimen of diet and/or iron supplements until the patient’s iron stores have returned to a normal or acceptable level. For patients having higher than normal red blood cell levels and/or hemoglobin levels, then administration of the one or more antagonists of the disclosure may be delayed until the levels have returned to a normal or acceptable level.

In certain embodiments, if one or more hematologic parameters are outside the normal range or on the high side of normal in a patient who is a candidate to be treated with one or more BMP antagonists, then the onset of administration may not be delayed. However, the dosage amount or frequency of dosing of the one or more antagonists of the disclosure may be set at an amount that would reduce the risk of an unacceptable increase in the hematologic parameters arising upon administration of the one or more antagonists of the disclosure. Alternatively, a therapeutic regimen may be developed for the patient that combines one or more BMP antagonists with a therapeutic agent that addresses the undesirable level of the hematologic parameter. For example, if the patient has elevated blood pressure, then a therapeutic regimen may be designed involving administration of one or more BMP antagonist agents and a blood pressure lowering agent. For a patient having lower than desired iron stores, a therapeutic regimen may be developed involving one or more BMP antagonists of the disclosure and iron supplementation.

In one embodiment, baseline parameter(s) for one or more hematologic parameters may be established for a patient who is a candidate to be treated with one or more BMP antagonists of the disclosure and an appropriate dosing regimen established for that patient based on the baseline value(s). Alternatively, established baseline parameters based on a patient’s medical history could be used to inform an appropriate antagonist dosing regimen for a patient. For example, if a healthy patient has an established baseline blood pressure reading that is above the defined normal range it may not be necessary to bring the patient’s blood pressure into the range that is considered normal for the general population prior to treatment with the one or more antagonist of the disclosure. A patient’s baseline values for one or more hematologic parameters prior to treatment with one or more BMP antagonists of the disclosure may also be used as the relevant comparative values for monitoring any changes to the hematologic parameters during treatment with the one or more antagonists of the disclosure.

In certain embodiments, one or more hematologic parameters are measured in patients who are being treated with one or more BMP antagonists. The hematologic parameters may be used to monitor the patient during treatment and permit adjustment or termination of the dosing with the one or more antagonists of the disclosure or additional dosing with another therapeutic agent. For example, if administration of one or more BMP antagonists results in an increase in blood pressure, red blood cell level, or hemoglobin level, or a reduction in iron stores, then the dose of the one or more antagonists of the disclosure may be reduced in amount or frequency in order to decrease the effects of the one or more antagonists of the disclosure on the one or more hematologic parameters. If administration of one or more BMP antagonists results in a change in one or more hematologic parameters that is adverse to the patient, then the dosing of the one or more antagonists of the disclosure may be terminated either temporarily, until the hematologic parameter(s) return to an acceptable level, or permanently. Similarly, if one or more hematologic parameters are not brought within an acceptable range after reducing the dose or frequency of administration of the one or more antagonists of the disclosure, then the dosing may be terminated. As an alternative, or in addition to, reducing or terminating the dosing with the one or more antagonists of the disclosure, the patient may be dosed with an additional therapeutic agent that addresses the undesirable level in the hematologic parameter(s), such as, for example, a blood pressure lowering agent or an iron supplement. For example, if a patient being treated with one or more BMP antagonists has elevated blood pressure, then dosing with the one or more antagonists of the disclosure may continue at the same level and a blood-pressure-lowering agent is added to the treatment regimen, dosing with the one or more antagonist of the disclosure may be reduced (e.g., in amount and/or frequency) and a blood-pressure-lowering agent is added to the treatment regimen, or dosing with the one or more antagonist of the disclosure may be terminated and the patient may be treated with a blood-pressure-lowering agent.

### 10. Pharmaceutical Compositions

In certain aspects, BMP antagonists, or combinations of such antagonists, of the present disclosure can be administered alone or as a component of a pharmaceutical formulation (also referred to as a therapeutic composition or pharmaceutical composition). A pharmaceutical formation refers to a preparation which is in such form as to permit the biological activity of an active ingredient (e.g., an agent of the present disclosure) contained therein to be effective and which contains no additional components which are unacceptably toxic to a subject to which the formulation would be administered. The subject compounds may be formulated for administration in any convenient way for use in human or veterinary medicine. For example, one or more agents of the present disclosure may be formulated with a pharmaceutically acceptable carrier. A pharmaceutically acceptable carrier refers to an ingredient in a pharmaceutical formulation, other than an active ingredient, which is generally nontoxic to a subject. A pharmaceutically acceptable carrier includes, but is not limited to, a buffer, excipient, stabilizer, and/or preservative. In general, pharmaceutical formulations for use in the present disclosure are in a pyrogen-free, physiologically-acceptable form when administered to a subject. Therapeutically useful agents other than those described herein, which may optionally be included in the formulation as described above, may be administered in combination with the subject agents in the methods of the present disclosure.

In certain embodiments, compositions will be administered parenterally [e.g., by intravenous (I.V.) injection, intraarterial injection, intraosseous injection, intramuscular injection, intrathecal injection, subcutaneous injection, or intradermal injection]. Pharmaceutical compositions suitable for parenteral administration may comprise one or more agents of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use. Injectable solutions or dispersions may contain antioxidants, buffers, bacteriostats, suspending agents, thickening agents, or solutes which render the formulation isotonic with the blood of the intended recipient. Examples of suitable aqueous and nonaqueous carriers which may be employed in the pharmaceutical formulations of the present disclosure include water, ethanol, polyols (e.g., glycerol, propylene glycol, polyethylene glycol, etc.), vegetable oils (e.g., olive oil), injectable organic esters (e.g., ethyl oleate), and suitable mixtures thereof. Proper fluidity can be maintained, for example, by the use of coating materials (e.g., lecithin), by the maintenance of the required particle size in the case of dispersions, and by the use of surfactants.

In some embodiments, compounds will be administered to the heart including, e.g., by intra-cardial administration, intra-pericardial administration, or by implant or device. For example, access to the pericardial space may be accomplished from outside the body by making a thoracic or subxiphoid incision to access and cut or pierce the pericardial sac. Access to the pericardial space from the exterior of the body, accomplished by passing a cannula or catheter type device through the chest wall and thereafter passing the cannula or catheter or a further instrument through the pericardium into the pericardial space, is disclosed, for example, in U.S. Pat. Nos. 5,336,252, 5,827,216, 5,900,433, 5,972,013, 6,162,195, 6,206,004, and 6,592,552. In certain cases the pericardial sac may be cut by a cutting instrument as disclosed, for example, in U.S. Pat. Nos. 5,931,810, 6,156,009, and 6,231,518.

In some embodiments, a therapeutic method of the present disclosure includes administering the pharmaceutical composition systemically, or locally, from an implant or device. Further, the pharmaceutical composition may be encapsulated or injected in a form for delivery to a target tissue site (e.g., bone marrow or muscle). In certain embodiments, compositions of the present disclosure may include a matrix capable of delivering one or more of the agents of the present disclosure to a target tissue site (e.g., bone marrow or muscle), providing a structure for the developing tissue and optimally capable of being resorbed into the body. For example, the matrix may provide slow release of one or more agents of the present disclosure. Such matrices may be formed of materials presently in use for other implanted medical applications.

The choice of matrix material may be based on one or more of: biocompatibility, biodegradability, mechanical properties, cosmetic appearance, and interface properties. The particular application of the subject compositions will define the appropriate formulation. Potential matrices for the compositions may be biodegradable and chemically defined calcium sulfate, tricalciumphosphate, hydroxyapatite, polylactic acid, and polyanhydrides. Other potential materials are biodegradable and biologically well-defined including, for example, bone or dermal collagen. Further matrices are comprised of pure proteins or extracellular matrix components. Other potential matrices are non-biodegradable and chemically defined including, for example, sintered hydroxyapatite, bioglass, aluminates, or other ceramics. Matrices may be comprised of combinations of any of the above mentioned types of material including, for example, polylactic acid and hydroxyapatite or collagen and tricalciumphosphate. The bioceramics may be altered in composition (e.g., calcium-aluminate-phosphate) and processing to alter one or more of pore size, particle size, particle shape, and biodegradability.

In certain embodiments, pharmaceutical compositions of present disclosure can be administered topically. “Topical application” or “topically” means contact of the pharmaceutical composition with body surfaces including, for example, the skin, wound sites, and mucous membranes. The topical pharmaceutical compositions can have various application forms and typically comprises a drug-containing layer, which is adapted to be placed near to or in direct contact with the tissue upon topically administering the composition. Pharmaceutical compositions suitable for topical administration may comprise one or more one or more BMP antagonists of the disclosure in combination formulated as a liquid, a gel, a cream, a lotion, an ointment, a foam, a paste, a putty, a semi-solid, or a solid. Compositions in the liquid, gel, cream, lotion, ointment, foam, paste, or putty form can be applied by spreading, spraying, smearing, dabbing or rolling the composition on the target tissue. The compositions also may be impregnated into sterile dressings, transdermal patches, plasters, and bandages. Compositions of the putty, semi-solid or solid forms may be deformable. They may be elastic or non-elastic (e.g., flexible or rigid). In certain aspects, the composition forms part of a composite and can include fibers, particulates, or multiple layers with the same or different compositions.

Topical compositions in the liquid form may include pharmaceutically acceptable solutions, emulsions, microemulsions, and suspensions. In addition to the active ingredient(s), the liquid dosage form may contain an inert diluent commonly used in the art including, for example, water or other solvent, a solubilizing agent and/or emulsifier [e.g., ethyl alcohol, isopropyl alcohol, ethyl carbonate, ethyl acetate, benzyl alcohol, benzyl benzoate, propylene glycol, or 1,3-butylene glycol, an oil (e.g., cottonseed, groundnut, corn, germ, olive, castor, and sesame oil), glycerol, tetrahydrofuryl alcohol, a polyethylene glycol, a fatty acid ester of sorbitan, and mixtures thereof].

Topical gel, cream, lotion, ointment, semi-solid or solid compositions may include one or more thickening agents, such as a polysaccharide, synthetic polymer or protein-based polymer. In one embodiment of the invention, the gelling agent herein is one that is suitably nontoxic and gives the desired viscosity. The thickening agents may include polymers, copolymers, and monomers of: vinylpyrrolidones, methacrylamides, acrylamides N-vinylimidazoles, carboxy vinyls, vinyl esters, vinyl ethers, silicones, polyethyleneoxides, polyethyleneglycols, vinylalcohols, sodium acrylates, acrylates, maleic acids, NN-dimethylacrylamides, diacetone acrylamides, acrylamides, acryloyl morpholine, pluronic, collagens, polyacrylamides, polyacrylates, polyvinyl alcohols, polyvinylenes, polyvinyl silicates, polyacrylates substituted with a sugar (e.g., sucrose, glucose, glucosamines, galactose, trehalose, mannose, or lactose), acylamidopropane sulfonic acids, tetramethoxyorthosilicates, methyltrimethoxyorthosilicates, tetraalkoxyorthosilicates, trialkoxyorthosilicates, glycols, propylene glycol, glycerine, polysaccharides, alginates, dextrans, cyclodextrin, celluloses, modified celluloses, oxidized celluloses, chitosans, chitins, guars, carrageenans, hyaluronic acids, inulin, starches, modified starches, agarose, methylcelluloses, plant gums, hylaronans, hydrogels, gelatins, glycosaminoglycans, carboxymethyl celluloses, hydroxyethyl celluloses, hydroxy propyl methyl celluloses, pectins, low-methoxy pectins, cross-linked dextrans, starch-acrylonitrile graft copolymers, starch sodium polyacrylate, hydroxyethyl methacrylates, hydroxyl ethyl acrylates, polyvinylene, polyethylvinylethers, polymethyl methacrylates, polystyrenes, polyurethanes, polyalkanoates, polylactic acids, polylactates, poly(3-hydroxybutyrate), sulfonated hydrogels, AMPS (2-acrylamido-2-methyl-1-propanesulfonic acid), SEM (sulfoethylmethacrylate), SPM (sulfopropyl methacrylate), SPA (sulfopropyl acrylate), N,N-dimethyl-N-methacryloxyethyl-N-(3-sulfopropyl)ammonium betaine, methacryllic acid amidopropyl-dimethyl ammonium sulfobetaine, SPI (itaconic acid-bis(1-propyl sulfonizacid-3) ester di-potassium salt), itaconic acids, AMBC (3-acrylamido-3-methylbutanoic acid), beta-carboxyethyl acrylate (acrylic acid dimers), and maleic anhydride-methylvinyl ether polymers, derivatives thereof, salts thereof, acids thereof, and combinations thereof. In certain embodiments, pharmaceutical compositions of present disclosure can be administered orally, for example, in the form of capsules, cachets, pills, tablets, lozenges (using a flavored basis such as sucrose and acacia or tragacanth), powders, granules, a solution or a suspension in an aqueous or non-aqueous liquid, an oil-in-water or water-in-oil liquid emulsion, or an elixir or syrup, or pastille (using an inert base, such as gelatin and glycerin, or sucrose and acacia), and/or a mouth wash, each containing a predetermined amount of a compound of the present disclosure and optionally one or more other active ingredients. A compound of the present disclosure and optionally one or more other active ingredients may also be administered as a bolus, electuary, or paste.

In solid dosage forms for oral administration (e.g., capsules, tablets, pills, dragees, powders, and granules), one or more compounds of the present disclosure may be mixed with one or more pharmaceutically acceptable carriers including, for example, sodium citrate, dicalcium phosphate, a filler or extender (e.g., a starch, lactose, sucrose, glucose, mannitol, and silicic acid), a binder (e.g. carboxymethylcellulose, an alginate, gelatin, polyvinyl pyrrolidone, sucrose, and acacia), a humectant (e.g., glycerol), a disintegrating agent (e.g., agar-agar, calcium carbonate, potato or tapioca starch, alginic acid, a silicate, and sodium carbonate), a solution retarding agent (e.g. paraffin), an absorption accelerator (e.g. a quaternary ammonium compound), a wetting agent (e.g., cetyl alcohol and glycerol monostearate), an absorbent (e.g., kaolin and bentonite clay), a lubricant (e.g., a talc, calcium stearate, magnesium stearate, solid polyethylene glycols, sodium lauryl sulfate), a coloring agent, and mixtures thereof. In the case of capsules, tablets, and pills, the pharmaceutical formulation (composition) may also comprise a buffering agent. Solid compositions of a similar type may also be employed as fillers in soft and hard-filled gelatin capsules using one or more excipients including, e.g., lactose or a milk sugar as well as a high molecular-weight polyethylene glycol.

Liquid dosage forms for oral administration of the pharmaceutical composition may include pharmaceutically acceptable emulsions, microemulsions, solutions, suspensions, syrups, and elixirs. In addition to the active ingredient(s), the liquid dosage form may contain an inert diluent commonly used in the art including, for example, water or other solvent, a solubilizing agent and/or emulsifier [e.g., ethyl alcohol, isopropyl alcohol, ethyl carbonate, ethyl acetate, benzyl alcohol, benzyl benzoate, propylene glycol, or 1,3-butylene glycol, an oil (e.g., cottonseed, groundnut, corn, germ, olive, castor, and sesame oil), glycerol, tetrahydrofuryl alcohol, a polyethylene glycol, a fatty acid ester of sorbitan, and mixtures thereof]. Besides inert diluents, the oral formulation can also include an adjuvant including, for example, a wetting agent, an emulsifying and suspending agent, a sweetening agent, a flavoring agent, a coloring agent, a perfuming agent, a preservative agent, and combinations thereof.

Suspensions, in addition to the active compounds, may contain suspending agents including, for example, an ethoxylated isostearyl alcohol, polyoxyethylene sorbitol, a sorbitan ester, microcrystalline cellulose, aluminum metahydroxide, bentonite, agar-agar, tragacanth, and combinations thereof.

Prevention of the action and/or growth of microorganisms may be ensured by the inclusion of various antibacterial and antifungal agents including, for example, paraben, chlorobutanol, and phenol sorbic acid.

In certain embodiments, it may be desirable to include an isotonic agent including, for example, a sugar or sodium chloride into the compositions. In addition, prolonged absorption of an injectable pharmaceutical form may be brought about by the inclusion of an agent that delay absorption including, for example, aluminum monostearate and gelatin.

It is understood that the dosage regimen will be determined by the attending physician considering various factors which modify the action of the one or more of the agents of the present disclosure. In the case of a BMP antagonist that promotes red blood cell formation, various factors may include, but are not limited to, the patient’s red blood cell count, hemoglobin level, the desired target red blood cell count, the patient’s age, the patient’s sex, the patient’s diet, the severity of any disease that may be contributing to a depressed red blood cell level, the time of administration, and other clinical factors. The addition of other known active agents to the final composition may also affect the dosage. Progress can be monitored by periodic assessment of one or more of red blood cell levels, hemoglobin levels, reticulocyte levels, and other indicators of the hematopoietic process.

In certain embodiments, the present disclosure also provides gene therapy for the in vivo production of one or more of the agents of the present disclosure. Such therapy would achieve its therapeutic effect by introduction of the agent sequences into cells or tissues having one or more of the disorders as listed above. Delivery of the agent sequences can be achieved, for example, by using a recombinant expression vector such as a chimeric virus or a colloidal dispersion system. Preferred therapeutic delivery of one or more of agent sequences of the disclosure is the use of targeted liposomes.

Various viral vectors which can be utilized for gene therapy as taught herein include adenovirus, herpes virus, vaccinia, or an RNA virus (e.g., a retrovirus). The retroviral vector may be a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing one or more of the agents of the present disclosure.

Alternatively, tissue culture cells can be directly transfected with plasmids encoding the retroviral structural genes (gag, pol, and env), by conventional calcium phosphate transfection. These cells are then transfected with the vector plasmid containing the genes of interest. The resulting cells release the retroviral vector into the culture medium.

Another targeted delivery system for one or more of the agents of the present disclosure is a colloidal dispersion system. Colloidal dispersion systems include, for example, macromolecule complexes, nanocapsules, microspheres, beads, and lipid-based systems including oil-in-water emulsions, micelles, mixed micelles, and liposomes. In certain embodiments, the preferred colloidal system of this disclosure is a liposome. Liposomes are artificial membrane vesicles which are useful as delivery vehicles in vitro and in vivo. RNA, DNA, and intact virions can be encapsulated within the aqueous interior and be delivered to cells in a biologically active form [Fraley, et al. (1981) Trends Biochem. Sci., 6:77]. Methods for efficient gene transfer using a liposome vehicle are known in the art [Mannino, et al. (1988) Biotechniques, 6:682, 1988].

The composition of the liposome is usually a combination of phospholipids, which may include a steroid (e.g.cholesterol). The physical characteristics of liposomes depend on pH, ionic strength, and the presence of divalent cations. Other phospholipids or other lipids may also be used including, for example a phosphatidyl compound (e.g., phosphatidylglycerol, phosphatidylcholine, phosphatidylserine, phosphatidylethanolamine, a sphingolipid, a cerebroside, and a ganglioside), egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

### EXEMPLIFICATION

The invention now being generally described, it will be more readily understood by reference to the following examples, which are included merely for purposes of illustration of certain embodiments of the present invention, and are not intended to limit the invention.

### Example 1. Generation of a BMPRII-Fc Fusion Protein

A homodimeric BMPRII-Fc fusion protein comprising the extracellular domain of human BMPRII fused to a human immunoglobulin G1 Fc domain with a linker was generated. Leader sequences for use with BMPRII-Fc fusion polypeptide include the native human BMPRII precursor leader, MTSSLQRPWRVPWLPWTILLVSTAAA (SEQ ID NO: 43), and the tissue plasminogen activator (TPA) leader.

The human BMPRII-Fc polypeptide sequence (SEQ ID NO: 44) with a TPA leader is shown below:

1 MDAMKRGLCC VLLLCGAVFV SPGASQNQER LCAFKDPYQQ DLGIGESRIS
51 HENGTILCSK GSTCYGLWEK SKGDINLVKQ GCWSHIGDPQ ECHYEECVVT
101 TTPPSIQNGT YRFCCCSTDL CNVNFTENFP PPDTTPLSPP HSFNRDETGG
151 GTHTCPPCPA PELLGGPSVF LFPPKPKDTL MISRTPEVTC VVVDVSHEDP
201 EVKFNWYVDG VEVHNAKTKP REEQYNSTYR VVSVLTVLHQ DWLNGKEYKC
251 KVSNKALPAP IEKTISKAKG QPREPQVYTL PPSREEMTKN QVSLTCLVKG
301 FYPSDIAVEW ESNGQPENNY KTTPPVLDSD GSFFLYSKLT VDKSRWQQGN
351 VFSCSVMHEA LHNHYTQKSL SLSPGK (SEQ ID NO: 44)

The leader sequence and linker are underlined. The amino acid sequence of SEQ ID NO: 44 may optionally be provided with lysine removed from the C-terminus.

This BMPRII-Fc fusion protein is encoded by the following nucleic acid sequence (SEQ ID NO: 45):

1 ATGGATGCAA TGAAGAGAGG GCTCTGCTGT GTGCTGCTGC TGTGTGGAGC
51 AGTCTTCGTT TCGCCCGGCG CCTCGCAGAA TCAAGAACGC CTATGTGCGT
101 TTAAAGATCC GTATCAGCAA GACCTTGGGA TAGGTGAGAG TAGAATCTCT
151 CATGAAAATG GGACAATATT ATGCTCGAAA GGTAGCACCT GCTATGGCCT
201 TTGGGAGAAA TCAAAAGGGG ACATAAATCT TGTAAAACAA GGATGTTGGT
251 CTCACATTGG AGATCCCCAA GAGTGTCACT ATGAAGAATG TGTAGTAACT
301 ACCACTCCTC CCTCAATTCA GAATGGAACA TACCGTTTCT GCTGTTGTAG
351 CACAGATTTA TGTAATGTCA ACTTTACTGA GAATTTTCCA CCTCCTGACA
401 CAACACCACT CAGTCCACCT CATTCATTTA ACCGAGATGA GACCGGTGGT
451 GGAACTCACA CATGCCCACC GTGCCCAGCA CCTGAACTCC TGGGGGGACC
501 GTCAGTCTTC CTCTTCCCCC CAAAACCCAA GGACACCCTC ATGATCTCCC
551 GGACCCCTGA GGTCACATGC GTGGTGGTGG ACGTGAGCCA CGAAGACCCT
601 GAGGTCAAGT TCAACTGGTA CGTGGACGGC GTGGAGGTGC ATAATGCCAA
651 GACAAAGCCG CGGGAGGAGC AGTACAACAG CACGTACCGT GTGGTCAGCG
701 TCCTCACCGT CCTGCACCAG GACTGGCTGA ATGGCAAGGA GTACAAGTGC
751 AAGGTCTCCA ACAAAGCCCT CCCAGCCCCC ATCGAGAAAA CCATCTCCAA
801 AGCCAAAGGG CAGCCCCGAG AACCACAGGT GTACACCCTG CCCCCATCCC
851 GGGAGGAGAT GACCAAGAAC CAGGTCAGCC TGACCTGCCT GGTCAAAGGC
901 TTCTATCCCA GCGACATCGC CGTGGAGTGG GAGAGCAATG GGCAGCCGGA
951 GAACAACTAC AAGACCACGC CTCCCGTGCT GGACTCCGAC GGCTCCTTCT
1001 TCCTCTATAG CAAGCTCACC GTGGACAAGA GCAGGTGGCA GCAGGGGAAC
1051 GTCTTCTCAT GCTCCGTGAT GCATGAGGCT CTGCACAACC ACTACACGCA
1101 GAAGAGCCTC TCCCTGTCTC CGGGTAAA (SEQ ID NO: 45)

A processed BMPRII-Fc fusion polypeptide (SEQ ID NO: 46) is as follows and may optionally be provided with lysine removed from the C-terminus.

1 SQNQERLCAF KDPYQQDLGI GESRISHENG TILCSKGSTC YGLWEKSKGD
51 INLVKQGCWS HIGDPQECHY EECVVTTTPP SIQNGTYRFC CCSTDLCNVN
101 FTENFPPPDT TPLSPPHSFN RDETGGGTHT CPPCPAPELL GGPSVFLFPP
151 KPKDTLMISR TPEVTCVVVD VSHEDPEVKF NWYVDGVEVH NAKTKPREEQ
201 YNSTYRVVSV LTVLHQDWLN GKEYKCKVSN KALPAPIEKT ISKAKGQPRE
251 PQVYTLPPSR EEMTKNQVSL TCLVKGFYPS DIAVEWESNG QPENNYKTTP
301 PVLDSDGSFF LYSKLTVDKS RWQQGNVFSC SVMHEALHNH YTQKSLSLSP
351 GK (SEQ ID NO: 46)

This BMPRII-Fc fusion protein is encoded by the following nucleic acid sequence (SEQ ID NO: 47):

1 TCGCAGAATC AAGAACGCCT ATGTGCGTTT AAAGATCCGT ATCAGCAAGA
51 CCTTGGGATA GGTGAGAGTA GAATCTCTCA TGAAAATGGG ACAATATTAT
101 GCTCGAAAGG TAGCACCTGC TATGGCCTTT GGGAGAAATC AAAAGGGGAC
151 ATAAATCTTG TAAAACAAGG ATGTTGGTCT CACATTGGAG ATCCCCAAGA
201 GTGTCACTAT GAAGAATGTG TAGTAACTAC CACTCCTCCC TCAATTCAGA
251 ATGGAACATA CCGTTTCTGC TGTTGTAGCA CAGATTTATG TAATGTCAAC
301 TTTACTGAGA ATTTTCCACC TCCTGACACA ACACCACTCA GTCCACCTCA
351 TTCATTTAAC CGAGATGAGA CCGGTGGTGG AACTCACACA TGCCCACCGT
401 GCCCAGCACC TGAACTCCTG GGGGGACCGT CAGTCTTCCT CTTCCCCCCA
451 AAACCCAAGG ACACCCTCAT GATCTCCCGG ACCCCTGAGG TCACATGCGT
501 GGTGGTGGAC GTGAGCCACG AAGACCCTGA GGTCAAGTTC AACTGGTACG
551 TGGACGGCGT GGAGGTGCAT AATGCCAAGA CAAAGCCGCG GGAGGAGCAG
601 TACAACAGCA CGTACCGTGT GGTCAGCGTC CTCACCGTCC TGCACCAGGA
651 CTGGCTGAAT GGCAAGGAGT ACAAGTGCAA GGTCTCCAAC AAAGCCCTCC
701 CAGCCCCCAT CGAGAAAACC ATCTCCAAAG CCAAAGGGCA GCCCCGAGAA
751 CCACAGGTGT ACACCCTGCC CCCATCCCGG GAGGAGATGA CCAAGAACCA
801 GGTCAGCCTG ACCTGCCTGG TCAAAGGCTT CTATCCCAGC GACATCGCCG
851 TGGAGTGGGA GAGCAATGGG CAGCCGGAGA ACAACTACAA GACCACGCCT
901 CCCGTGCTGG ACTCCGACGG CTCCTTCTTC CTCTATAGCA AGCTCACCGT
951 GGACAAGAGC AGGTGGCAGC AGGGGAACGT CTTCTCATGC TCCGTGATGC
1001 ATGAGGCTCT GCACAACCAC TACACGCAGA AGAGCCTCTC CCTGTCTCCG
1051 GGTAAA (SEQ ID NO: 47)

The BMPRII-Fc fusion polypeptide of SEQ ID NO: 46 may be expressed and purified from a CHO cell line to give rise to a homodimeric BMPRII-Fc fusion protein complex.

Purification of various BMPRII-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

A Biacore™-based binding assay was used to determine the ligand binding selectivity of the BMPRII-Fc protein complex described above. The BMPRII-Fc homodimer was captured onto the system using an anti-Fc antibody, and ligands were injected and allowed to flow over the captured receptor protein. Results are summarized in the table below.

Ligand binding profile of BMPRII-Fc homodimer

| Ligand | kₐ (1/Ms) | k_{d} (1/s) | K_{D} (pM) |
|---|---|---|---|
| BMP 10 | 2.6 x10⁷ | 2.5 x10⁻³ | 100 |
| BMP9 | 1.2 x10⁷ | 2.6 x10⁻² | 2100 |
| BMP6 | Transient* | | 8900 |

* Indeterminate due to transient nature of interaction

These ligand binding data demonstrate that homodimeric BMPRII-Fc fusion protein binds with high picomolar affinity to BMP10 and with approximately ten-fold lower affinity to BMP9. As ligand traps, BMPRII-Fc polypeptides should preferably exhibit a slow rate of ligand dissociation, so the off-rates observed for BMP10 in particular is desirable.

Surprisingly, despite literature suggesting that BMPRII acts as the major type II receptor for canonical BMP proteins such as BMP2, BMP4, BMP6 or BMP7, BMPRII-Fc fusion protein shows no substantial binding to any of BMP2, BMP4, BMP6 or BMP7. Accordingly, homodimeric BMPRII-Fc will be useful in certain therapeutic applications where antagonism of BMP 10 and BMP9 is advantageous.

### Example 2: ALK1-Fc Fusion Proteins

An ALK1 fusion protein was generated that has the extracellular domain of human ALK1 fused to a human Fc or mouse ALK1 fused to a murine Fc domain with a linker in between. The constructs are referred to as ALK1-hFc and mALK1-mFc, respectively.

Notably, while the conventional C-terminus of the extracellular domain of human ALK1 protein is amino acid 118 of SEQ ID NO: 11, we have determined that it is desirable to avoid having a domain that ends at a glutamine residue. Accordingly, the portion of SEQ ID NO: 50 that derives from human ALK1 incorporates two residues C-terminal to Q118, a leucine and an alanine. The disclosure therefore provides ALK1 ECD polypeptides (including Fc fusion proteins) having a C-terminus of the ALK1 derived sequence that is anywhere from 1 to 5 amino acids upstream (113-117 relative to SEQ ID NO: 11) or downstream (119-123) of Q118.

The ALK1-hFc and ALK1-mFc proteins were expressed in CHO cell lines. Three different leader sequences were considered: (i) Honey bee mellitin (HBML), (ii) Tissue Plasminogen Activator (TPA), and Native: MTLGSPRKGLLMLLMALVTQG (SEQ ID NO: 48).

The selected ALK1-hFc form employs the TPA leader and has the unprocessed amino acid sequence shown in below:

MDAMKRGLCCVLLLCGAVFVSPGADPVKPSRGPLVTCTCESPHCKGPTCR GAWCTVVLVREEGRHPOEHRGCGNLHRELCRGRPTEFVNHYCCDSHLCNH NVSLVLEATQPPSEOPGTDGOLATGGGTHTCPPCPAPEALGAPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPVPIEKTISKAKGQPRE
PQVYTLPPSREEMTKNQVSLTCLVKGFYPSDIAVEWESNGQPENNYKTTP
PVLDSDGPFFLYSKLTVDKSRWQQGNVFSCSVMHEALHNHYTQKSLSLSP
GK (SEQ ID NO: 49)

The ALK1 extracellular domain is underlined.

The ALK1-hFc as purified from CHO cell lines is shown below:

DPVKPSRGPLVTCTCESPHCKGPTCRGAWCTVVLVREEGRHPQEHRGCGN
LHRELCRGRPTEFVNHYCCDSHLCNHNVSLVLEATQPPSEQPGTDGQLAT
GGGTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPVPIEKTISKAKGQPREPQVYTLPPSREEMTKNQVSLTCLV
KGFYPSDIAVEWESNGQPENNYKTTPPVLDSDGSFFLYSKLTVDKSRWQQ
GNVFSCSVMHEALHNHYTQKSLSLSPGK (SEQ ID NO: 50)

Purification can be achieved by a series of column chromatography steps, including, for example, three or more of the following, in any order: protein A chromatography, Q sepharose chromatography, phenylsepharose chromatography, size exclusion chromatography, and cation exchange chromatography. The purification can be completed with viral filtration and buffer exchange. The ALK1-hFc protein was purified to a purity of >98% as determined by size exclusion chromatography and >95% as determined by SDS PAGE.

Affinities of several ligands for ALK1-hFc were evaluated in vitro with a Biacore™ instrument. Various ligands were immobilized on a Biacore™ CM5 chip using standard amine-coupling procedure. ALK1-hFc was loaded onto the system, and binding was measured. ALK1-hFc bound to BMP10 with a dissociation constant (K_{D}) of 1.49 × 10⁻¹¹ M and BMP9 with a K_{D} of 3.14 × 10⁻¹¹ M. ALK1-hFc had no detectable binding affinity for BMP5 or BMP6. ALK1-mFc behaved similarly.

### Example 3: Generation of ENG-Fc Fusion Proteins

Endoglin (ENG) fusion protein [hENG(26-586)-hFc] in which the full-length extracellular domain (ECD) of human ENG (amino acids 26-586 of SEQ ID NO: 15) was attached to a human IgG1 Fc domain with a linker between these domains.

Three different leader sequences were considered: (i) Honey bee mellitin (HBML), (ii) Tissue plasminogen activator (TPA), and (iii) native human ENG:

MDRGTLPLAVALLLASCSLSPTSLA (SEQ ID NO: 51)

The selected form of hENG(26-586)-hFc uses the TPA leader, has the unprocessed amino acid sequence shown in below:

1 MDAMKRGLCC VLLLCGAVFV SPGAETVHCD LQPVGPERDE VTYTTSQVSK
51 GCVAQAPNAI LEVHVLFLEF PTGPSQLELT LQASKQNGTW PREVLLVLSV
101 NSSVFLHLQA LGIPLHLAYN SSLVTFQEPP GVNTTELPSF PKTQILEWAA
151 ERGPITSAAE LNDPQSILLR LGQAQGSLSF CMLEASQDMG RTLEWRPRTP
201 ALVRGCHLEG VAGHKEAHIL RVLPGHSAGP RTVTVKVELS CAPGDLDAVL
251 ILQGPPYVSW LIDANHNMQI WTTGEYSFKI FPEKNIRGFK LPDTPQGLLG
301 EARMLNASIV ASFVELPLAS IVSLHASSCG GRLQTSPAPI QTTPPKDTCS
351 PELLMSLIQT KCADDAMTLV LKKELVAHLK CTITGLTFWD PSCEAEDRGD
401 KFVLRSAYSS CGMQVSASMI SNEAVVNILS SSSPQRKKVH CLNMDSLSFQ
451 LGLYLSPHFL QASNTIEPGQ QSFVQVRVSP SVSEFLLQLD SCHLDLGPEG
501 GTVELIQGRA AKGNCVSLLS PSPEGDPRFS FLLHFYTVPI PKTGTLSCTV
551 ALRPKTGSQD QEVHRTVFMR LNIISPDLSG CTSKGTGGGP KSCDKTHTCP
601 PCPAPELLGG PSVFLFPPKP KDTLMISRTP EVTCVVVDVS HEDPEVKFNW
651 YVDGVEVHNA KTKPREEQYN STYRVVSVLT VLHQDWLNGK EYKCKVSNKA
701 LPAPIEKTIS KAKGQPREPQ VYTLPPSREE MTKNQVSLTC LVKGFYPSDI
751 AVEWESNGQP ENNYKTTPPV LDSDGSFFLY SKLTVDKSRW QQGNVFSCSV
801 MHEALHNHYT QKSLSLSPGK (SEQ ID NO: 52)

The ENG extracellular domain is denoted with a single underline; the TPA leader is denoted with a double underline.

The hENG(26-586)-hFc described above is encoded by the nucleotide sequence shown below:

1 ATGGATGCAA TGAAGAGAGG GCTCTGCTGT GTGCTGCTGC TGTGTGGAGC
AGTCTTCGTT TCGCCCGGCG CCGAAACAGT CCATTGTGAC CTTCAGCCTG
101 TGGGCCCCGA GAGGGACGAG GTGACATATA CCACTAGCCA GGTCTCGAAG
GGCTGCGTGG CTCAGGCCCC CAATGCCATC CTTGAAGTCC ATGTCCTCTT
201 CCTGGAGTTC CCAACGGGCC CGTCACAGCT GGAGCTGACT CTCCAGGCAT
CCAAGCAAAA TGGCACCTGG CCCCGAGAGG TGCTTCTGGT CCTCAGTGTA
301 AACAGCAGTG TCTTCCTGCA TCTCCAGGCC CTGGGAATCC CACTGCACTT
GGCCTACAAT TCCAGCCTGG TCACCTTCCA AGAGCCCCCG GGGGTCAACA
401 CCACAGAGCT GCCATCCTTC CCCAAGACCC AGATCCTTGA GTGGGCAGCT
GAGAGGGGCC CCATCACCTC TGCTGCTGAG CTGAATGACC CCCAGAGCAT
501 CCTCCTCCGA CTGGGCCAAG CCCAGGGGTC ACTGTCCTTC TGCATGCTGG
AAGCCAGCCA GGACATGGGC CGCACGCTCG AGTGGCGGCC GCGTACTCCA
601 GCCTTGGTCC GGGGCTGCCA CTTGGAAGGC GTGGCCGGCC ACAAGGAGGC
GCACATCCTG AGGGTCCTGC CGGGCCACTC GGCCGGGCCC CGGACGGTGA
701 CGGTGAAGGT GGAACTGAGC TGCGCACCCG GGGATCTCGA TGCCGTCCTC
ATCCTGCAGG GTCCCCCCTA CGTGTCCTGG CTCATCGACG CCAACCACAA
801 CATGCAGATC TGGACCACTG GAGAATACTC CTTCAAGATC TTTCCAGAGA
AAAACATTCG TGGCTTCAAG CTCCCAGACA CACCTCAAGG CCTCCTGGGG
901 GAGGCCCGGA TGCTCAATGC CAGCATTGTG GCATCCTTCG TGGAGCTACC
GCTGGCCAGC ATTGTCTCAC TTCATGCCTC CAGCTGCGGT GGTAGGCTGC
1001 AGACCTCACC CGCACCGATC CAGACCACTC CTCCCAAGGA CACTTGTAGC
CCGGAGCTGC TCATGTCCTT GATCCAGACA AAGTGTGCCG ACGACGCCAT
1101 GACCCTGGTA CTAAAGAAAG AGCTTGTTGC GCATTTGAAG TGCACCATCA
CGGGCCTGAC CTTCTGGGAC CCCAGCTGTG AGGCAGAGGA CAGGGGTGAC
1201 AAGTTTGTCT TGCGCAGTGC TTACTCCAGC TGTGGCATGC AGGTGTCAGC
AAGTATGATC AGCAATGAGG CGGTGGTCAA TATCCTGTCG AGCTCATCAC
1301 CACAGCGGAA AAAGGTGCAC TGCCTCAACA TGGACAGCCT CTCTTTCCAG
CTGGGCCTCT ACCTCAGCCC ACACTTCCTC CAGGCCTCCA ACACCATCGA
1401 GCCGGGGCAG CAGAGCTTTG TGCAGGTCAG AGTGTCCCCA TCCGTCTCCG
AGTTCCTGCT CCAGTTAGAC AGCTGCCACC TGGACTTGGG GCCTGAGGGA
1501 GGCACCGTGG AACTCATCCA GGGCCGGGCG GCCAAGGGCA ACTGTGTGAG
CCTGCTGTCC CCAAGCCCCG AGGGTGACCC GCGCTTCAGC TTCCTCCTCC
1601 ACTTCTACAC AGTACCCATA CCCAAAACCG GCACCCTCAG CTGCACGGTA
GCCCTGCGTC CCAAGACCGG GTCTCAAGAC CAGGAAGTCC ATAGGACTGT
1701 CTTCATGCGC TTGAACATCA TCAGCCCTGA CCTGTCTGGT TGCACAAGCA
AAGGCACCGG TGGTGGACCC AAATCTTGTG ACAAAACTCA CACATGCCCA
1801 CCGTGCCCAG CACCTGAACT CCTGGGGGGA CCGTCAGTCT TCCTCTTCCC
CCCAAAACCC AAGGACACCC TCATGATCTC CCGGACCCCT GAGGTCACAT
1901 GCGTGGTGGT GGACGTGAGC CACGAAGACC CTGAGGTCAA GTTCAACTGG
TACGTGGACG GCGTGGAGGT GCATAATGCC AAGACAAAGC CGCGGGAGGA
2001 GCAGTACAAC AGCACGTACC GTGTGGTCAG CGTCCTCACC GTCCTGCACC
AGGACTGGCT GAATGGCAAG GAGTACAAGT GCAAGGTCTC CAACAAAGCC
2101 CTCCCAGCCC CCATCGAGAA AACCATCTCC AAAGCCAAAG GGCAGCCCCG
AGAACCACAG GTGTACACCC TGCCCCCATC CCGGGAGGAG ATGACCAAGA
2201 ACCAGGTCAG CCTGACCTGC CTGGTCAAAG GCTTCTATCC CAGCGACATC
GCCGTGGAGT GGGAGAGCAA TGGGCAGCCG GAGAACAACT ACAAGACCAC
2301 GCCTCCCGTG CTGGACTCCG ACGGCTCCTT CTTCCTCTAT AGCAAGCTCA
CCGTGGACAA GAGCAGGTGG CAGCAGGGGA ACGTCTTCTC ATGCTCCGTG
2401 ATGCATGAGG CTCTGCACAA CCACTACACG CAGAAGAGCC TCTCCCTGTC
CCCGGGTAAA TGA (SEQ ID NO: 53)

The ENG extracellular domain is denoted with a single underline; the TPA leader is denoted with a double underline.

An alternative hENG(26-586)-hFc sequence with TPA leader comprising an N-terminally truncated hFc domain attached to hENG(26-586) by a T linker was also envisioned:

1 MDAMKRGLCC VLLLCGAVFV SPGAETVHCD LQPVGPERDE VTYTTSQVSK
51 GCVAQAPNAI LEVHVLFLEF PTGPSQLELT LQASKQNGTW PREVLLVLSV
101 NSSVFLHLQA LGIPLHLAYN SSLVTFQEPP GVNTTELPSF PKTQILEWAA
151 ERGPITSAAE LNDPQSILLR LGQAQGSLSF CMLEASQDMG RTLEWRPRTP
201 ALVRGCHLEG VAGHKEAHIL RVLPGHSAGP RTVTVKVELS CAPGDLDAVL
251 ILQGPPYVSW LIDANHNMQI WTTGEYSFKI FPEKNIRGFK LPDTPQGLLG
301 EARMLNASIV ASFVELPLAS IVSLHASSCG GRLQTSPAPI QTTPPKDTCS
351 PELLMSLIQT KCADDAMTLV LKKELVAHLK CTITGLTFWD PSCEAEDRGD
401 KFVLRSAYSS CGMQVSASMI SNEAVVNILS SSSPQRKKVH CLNMDSLSFQ
451 LGLYLSPHFL QASNTIEPGQ QSFVQVRVSP SVSEFLLQLD SCHLDLGPEG
501 GTVELIQGRA AKGNCVSLLS PSPEGDPRFS FLLHFYTVPI PKTGTLSCTV
551 ALRPKTGSQD QEVHRTVFMR LNIISPDLSG CTSKGTGGGT HTCPPCPAPE
601 LLGGPSVFLF PPKPKDTLMI SRTPEVTCVV VDVSHEDPEV KFNWYVDGVE
651 VHNAKTKPRE EQYNSTYRVV SVLTVLHQDW LNGKEYKCKV SNKALPAPIE
701 KTISKAKGQP REPQVYTLPP SREEMTKNQV SLTCLVKGFY PSDIAVEWES
751 NGQPENNYKT TPPVLDSDGS FFLYSKLTVD KSRWQQGNVF SCSVMHEALH
801 NHYTQKSLSL SPGK (SEQ ID NO: 54)

Purification was achieved using a variety of techniques, including, for example, filtration of conditioned media, followed by protein A chromatography, elution with low-pH (3.0) glycine buffer, sample neutralization, and dialysis against PBS. Purity of samples was evaluated by analytical size-exclusion chromatography, SDS-PAGE, silver staining, and Western blot. Analysis of mature protein confirmed the expected N-terminal sequence.

Considered a co-receptor, ENG is widely thought to function by facilitating the binding of TGF-01 and -3 to multi-protein complexes of type I and type II receptors. To investigate the possibility of direct ligand binding by isolated ENG, surface plasmon resonance (SPR) methodology (Biacore™ instrument) was used to screen for binding of captured proteins comprising the full-length extracellular domain of ENG to a variety of soluble human TGF-β family ligands.

| Ligand | Construct Binding | |
|---|---|---|
| | hENG(26-586)-hFc* | hENG(26-586)** |
| hBMP-2 | - | - |
| hBMP-2/7 | - | - |
| hBMP-7 | - | - |
| hBMP-9 | ++++ | ++++ |
| hBMP-10 | ++++ | ++++ |
| hTGF-β1 | - | - |
| hTGF-β2 | - | - |
| hTGF-β3 | - | - |
| hActivin A | - | - |

* [hBMP-9], [hBMP-10] = 2.5 nM; all other ligands tested at 100 nM
** [hBMP-9], [hBMP-10] = 2.5 nM; all other ligands tested at 25 nM

As shown in this table, binding affinity to hENG(26-586)-hFc was high (++++, K_{D} < 1 nM) for hBMP9 and hBMP10 as evaluated at low ligand concentrations. Even at concentrations 40-fold higher, binding of TGF-01, TGF-β2, TGF-β3, activin A, BMP2, and BMP7 to hENG(26-586)-hFc was undetectable (-). For this latter group of ligands, lack of direct binding to isolated ENG fusion protein is noteworthy because multiprotein complexes of type I and type II receptors have been shown to bind most of them better in the presence of ENG than in its absence. As also shown in the table above, similar results were obtained when ligands were screened for their ability to bind immobilized hENG(26-586) (R&D Systems, catalog #1097-EN), a human variant with no Fc domain. Characterization by SPR determined that captured hENG(26-586)-hFc binds soluble BMP9 with a K_{D} of 29 pM and soluble BMP10 with a K_{D} of 400 pM. Thus, selective high-affinity binding of BMP9 and BMP10 is a previously unrecognized property of the ENG extracellular domain.

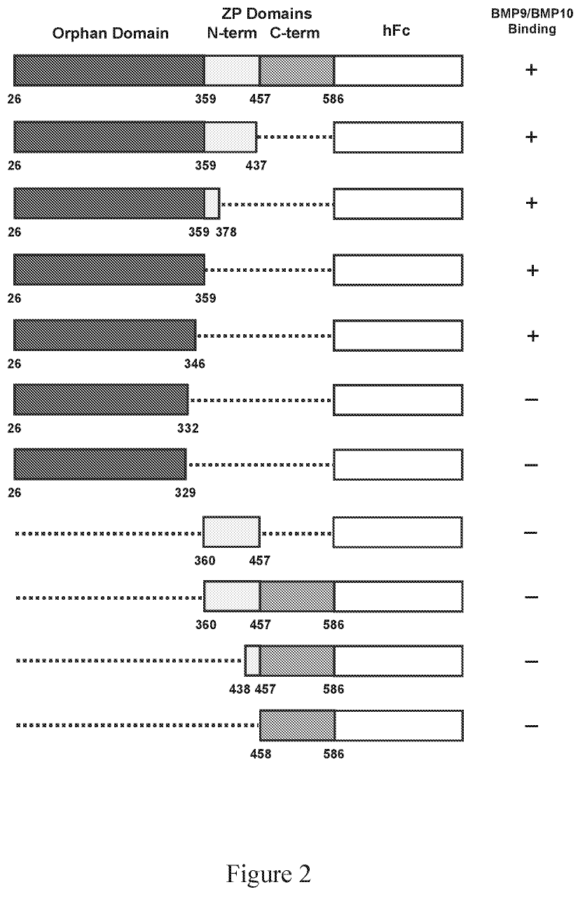

ENG fusion proteins in which truncated variants of the human ENG ECD were fused to a human IgG₁ Fc domain with a linker between where also generated. These variants are listed below, and the structures of selected variants are shown schematically in FIG. 2.

| | | | | |
|---|---|---|---|---|
| | Human Construct | Transient Expression | Purified | Stable Expression (CHO Cells) |
| Full Length | hENG(26-586)-hFc | HEK 293 | Yes | Yes |
| Carboxy-Terminal Truncations | hENG(26-581)-hFc | HEK 293 | Yes | No |
| | hENG(26-437)-hFc | HEK 293 | Yes | No |
| | hENG(26-378)-hFc | HEK 293 | Yes | No |
| | hENG(26-359)-hFc | HEK 293 | Yes | Yes |
| | hENG(26-346)-hFc | HEK 293 | Yes | Yes |
| | hENG(26-332)-hFc | HEK 293 | Yes | No |
| | hENG(26-329)-hFc | HEK 293 | Yes | No |
| | hENG(26-257)-hFc | HEK 293 | Yes | No |
| Amino-Terminal Truncations | hENG(360-586)-hFc | HEK 293 | Yes | No |
| | hENG(438-586)-hFc | HEK 293 | Yes | No |
| | hENG(458-586)-hFc | COS | No | No |
| Double Truncations | hENG(61-346)-hFc | HEK 293 | Yes | No |
| | hENG(129-346)-hFc | HEK 293 | Yes | No |
| | hENG(133-346)-hFc | HEK 293 | Yes | No |
| | hENG(166-346)-hFc | HEK 293 | Yes | No |
| | hENG(258-346)-hFc | HEK 293 | Yes | No |
| | hENG(360-581)-hFc | HEK 293 | Yes | No |
| | hENG(360-457)-hFc | COS | No | No |
| | hENG(3 60-43 7)-hFc | COS | No | No |
| | hENG(458-581)-hFc | COS | No | No |

These variants were expressed by transient transfection in HEK 293 cells or COS cells, as indicated.

SPR methodology was used to screen these hENG-hFc protein variants for high-affinity binding to human BMP9 and BMP10. In these experiments, captured hENG-hFc proteins were exposed to soluble BMP9 or BMP 10 at 100 nM each.

| | Human Construct | Binding to hBMP9 and hBMP10 |
|---|---|---|
| Full Length | hENG(26-586)-hFc | ++++ |
| Carboxy-Terminal Truncations | hENG(26-581)-hFc | ++++ |
| | hENG(26-437)-hFc | ++++ |
| | hENG(26-378)-hFc | ++++ |
| | hENG(26-359)-hFc | ++++ |
| | hENG(26-346)-hFc | ++++ |
| | hENG(26-332)-hFc | - |
| | hENG(26-329)-hFc | - |
| | hENG(26-257)-hFc | - |
| | hENG(360-586)-hFc | - |
| Amino-Terminal Truncations | hENG(438-586)-hFc | - |
| | hENG(458-586)-hFc | - |
| Double Truncations | hENG(61-346)-hFc | - |
| | hENG(129-346)-hFc | - |
| | hENG(133-346)-hFc | - |
| | hENG(166-346)-hFc | - |
| | hENG(258-346)-hFc | - |
| | hENG(360-581)-hFc | - |
| | hENG(360-457)-hFc | - |
| | hENG(360-437)-hFc | - |
| | hENG(458-581)-hFc | - |

++++ KD < 1 nM
- Binding undetectable

As indicated in the table above, high-affinity binding to BMP9 and BMP10 was observed only for the full-length construct and for C-terminally truncated variants as short as hENG(26-346)-hFc. High-affinity binding to BMP9 and BMP10 was lost for all N-terminal truncations of greater than 61 amino acids that were tested.

A panel of ligands were screened for potential binding to the C-terminal truncated variants hENG(26-346)-hFc, hENG(26-359)-hFc, and hENG(26-437)-hFc. High-affinity binding of these three proteins was selective for BMP9 and BMP10. Neither hENG(26-346)-hFc, hENG(26-359)-hFc, nor hENG(26-437)-hFc displayed detectable binding to BMP2, BMP7, TGFβ1, TGFβ2, TGFβ3, or activin A, even at high ligand concentrations.

| Ligand | Construct Binding | | |
|---|---|---|---|
| | hENG(26-346)-hFc* | hENG(26-359)-hFc** | hENG(26-437)-hFc** |
| hBMP-2 | - | - | - |
| hBMP-2/7 | - | - | - |
| hBMP-7 | - | - | - |
| hBMP-9 | ++++ | ++++ | ++++ |
| hBMP-10 | ++++ | ++++ | ++++ |
| hTGF-βI | - | - | - |
| hTGF-β2 | - | - | - |
| hTGF-β3 | - | - | - |
| hActivin A | - | - | - |

* [hBMP-9], [hBMP-10] = 5 nM; [hTGF-β3] = 50 nM; all other ligands tested at 100 nM
** [hBMP-9], [hBMP-10] = 5 nM; [hTGF-β3] = 50 nM; all other ligands tested at 100 nM
++++ KD < 1 nM
- Binding undetectable

SPR methodology was to compare the kinetics of BMP9 binding by five constructs: hENG(26-586)-hFc, hENG(26-437)-hFc, hENG(26-378)-hFc, hENG(26-359)-hFc, and hENG(26-346)-hFc. The affinity of human BMP-9 for hENG(26-359)-hFc or hENG(26-346)-hFc (with K_{DS} in the low picomolar range) was nearly an order of magnitude stronger than for the full-length construct. It is highly desirable for ligand traps such as ENG-Fc to exhibit a relatively slow rate of ligand dissociation, so the ten-fold improvement (decrease) in the BMP9 dissociation rate for hENG(26-346)-hFc compared to the full-length construct is particularly noteworthy.

| Ligand | Construct | K_{D} (x 10⁻¹² M) | k_{d} (x 10⁻⁴ s⁻¹) |
|---|---|---|---|
| hBMP-9 | hENG(26-586)-hFc * | 33 | 25 |
| | hENG(26-437)-hFc ** | 19 | 14 |
| | hENG(26-378)-hFc ** | 6.7 | 3.4 |
| | hENG(26-359)-hFc * | 4.2 | 3.5 |
| | hENG(26-346)-hFc * | 4.3 | 2.4 |

* CHO-cell-derived protein
** HEK293-cell-derived protein

As shown below, each of the truncated variants also bound BMP10 with higher affinity, and with better kinetics, compared to the full-length construct. Even so, the truncated variants differed in their degree of preference for BMP9 over BMP10 (based on K_{D} ratio), with hENG(26-346)-hFc displaying the largest differential and hENG(26-437)-hFC the smallest. This difference in degree of ligand preference among the truncated variants could potentially translate into meaningful differences in their activity in vivo.

| Ligand | Construct | K_{D} (x 10⁻¹² M) | k_{d} (x 10⁻⁴ s⁻¹) |
|---|---|---|---|
| hBMP-10 | hENG(26-586)-hFc * | 490 | 110 |
| | hENG(26-437)-hFc ** | 130 | 28 |
| | hENG(26-378)-hFc ** | 95 | 19 |
| | hENG(26-359)-hFc * | 86 | 23 |
| | hENG(26-346)-hFc * | 140 | 28 |

* CHO-cell-derived protein
** HEK293-cell-derived protein

The foregoing results indicate that fusion proteins comprising certain C-terminally truncated variants of the hENG ECD display high-affinity binding to BMP9 and BMP10 but not to a variety of other TGF-β family ligands, including TGFβ1 and TGFβ3. In particular, the truncated variants hENG(26-359)-hFc, hENG(26-346)-hFc, and hENG(26-378)-hFc display higher binding affinity at equilibrium and improved kinetic properties for BMP-9 compared to both the full-length construct hENG(26-586)-hFc and the truncated variant hENG(26-437)-hFc.

As disclosed above, N-terminal truncations as short as 36 amino acids (hENG(61-346)-hFc) were found to abolish ligand binding to ENG polypeptides. To anticipate the effect of even shorter N-terminal truncations on ligand binding, the secondary structure for the human endoglin orphan domain was predicted computationally with a modified Psipred version 3 (Jones, 1999, J Mol Biol 292:195-202). The analysis indicates that ordered secondary structure within the ENG polypeptide region defined by amino acids 26-60 of SEQ ID NO: 15 is limited to a four-residue beta strand predicted with high confidence at positions 42-45 of SEQ ID NO: 15 and a two-residue beta strand predicted with very low confidence at positions 28-29 of SEQ ID NO: 15. Accordingly, ENG polypeptide variants beginning at amino acids 27 or 28 and optionally those beginning at any of amino acids 29-42 of SEQ ID NO: 15 are likely to retain important structural elements and ligand binding.

For the mouse studies described below, an ENG-Fc fusion protein was constructed by fusing a truncated portion of the extracellular domain of human endoglin (i.e., amino acids 27-581) to an Fc domain of mouse IgG1 with a minimal linker (TGGG) positioned between the two domains. This construct is designated as hENG(27-581)-mFc and desmonstrated similar binding affinities as described above for hENG(26-581)-hFc.

### Example 4: BMP10 Propeptide Fusion Proteins

A BMP10 propeptide-Fc fusion protein comprising a C-terminal truncated, human BMP10 propeptide domain (amino acids 22-315 of SEQ ID NO: 1 fused to a human immunoglobulin G1 Fc domain with an optional linker) was generated. This fusion protein was designated as BMP10pro(22-315)-hFc. A similar fusion protein was generated using a mouse immunoglobulin G1 Fc domain, which is designated as BMP10pro(22-315)-mFc. Signal sequences for use with BMP10 propeptide-Fc fusion protein that were considered include, for example, the native human BMP10 precursor leader, MGSLVLTLCALFCLAAYLVSG (SEQ ID NO: 55), honeybee mellitin, and the tissue TPA leader.

The human BMP10pro(22-315)-hFc polypeptide sequence (SEQ ID NO: 56) with a TPA leader is shown below:

1 MDAMKRGLCC VLLLCGAVFV SPGASPIMNL EQSPLEEDMS LFGDVFSEQD
51 GVDFNTLLQS MKDEFLKTLN LSDIPTQDSA KVDPPEYMLE LYNKFATDRT
101 SMPSANIIRS FKNEDLFSQP VSFNGLRKYP LLFNVSIPHH EEVIMAELRL
151 YTLVQRDRMI YDGVDRKITI FEVLESKGDN EGERNMLVLV SGEIYGTNSE
201 WETFDVTDAI RRWQKSGSST HQLEVHIESK HDEAEDASSG RLEIDTSAQN
251 KHNPLLIVFS DDQSSDKERK EELNEMISHE QLPELDNLGL DSFSSGPGEE
301 ALLQMRSNII YDSTARIRTG GGTHTCPPCP APELLGGPSV FLFPPKPKDT
351 LMISRTPEVT CVVVDVSHED PEVKFNWYVD GVEVHNAKTK PREEQYNSTY
401 RVVSVLTVLH QDWLNGKEYK CKVSNKALPA PIEKTISKAK GQPREPQVYT
451 LPPSREEMTK NQVSLTCLVK GFYPSDIAVE WESNGQPENN YKTTPPVLDS
501 DGSFFLYSKL TVDKSRWQQG NVFSCSVMHE ALHNHYTQKS LSLSPGK (SEQ
ID NO: 56)

The BMP propeptide domain is underlined. The amino acid sequence of SEQ ID NO: 56 may optionally be provided with lysine removed from the C-terminus.

This BMP10pro(22-315)-hFc fusion protein is encoded by the following nucleic acid sequence (SEQ ID NO: 57):

1 ATGGATGCAA TGAAGAGAGG GCTCTGCTGT GTGCTGCTGC
TGTGTGGAGC
51 AGTCTTCGTT TCGCCCGGCG CCAGCCCCAT CATGAACCTA
GAGCAGTCTC
101 CTCTGGAAGA AGATATGTCC CTCTTTGGTG ATGTTTTCTC
AGAGCAAGAC
151 GGTGTCGACT TTAACACACT GCTCCAGAGC ATGAAGGATG
AGTTTCTTAA
201 GACACTAAAC CTCTCTGACA TCCCCACGCA GGATTCAGCC
AAGGTGGACC
251 CACCAGAGTA CATGTTGGAA CTCTACAACA AATTTGCAAC
AGATCGGACC
301 TCCATGCCCT CTGCCAACAT CATTAGGAGT TTCAAGAATG
AAGATCTGTT
351 TTCCCAGCCG GTCAGTTTTA ATGGGCTCCG AAAATACCCC
CTCCTCTTCA
401 ATGTGTCCAT TCCTCACCAT GAAGAGGTCA TCATGGCTGA
ACTTAGGCTA
451 TACACACTGG TGCAAAGGGA TCGTATGATA TACGATGGAG
TAGACCGGAA
501 AATTACCATT TTTGAAGTGC TGGAGAGCAA AGGGGATAAT
GAGGGAGAAA
551 GAAACATGCT GGTCTTGGTG TCTGGGGAGA TATATGGAAC
CAACAGTGAG
601 TGGGAGACTT TTGATGTCAC AGATGCCATC AGACGTTGGC
AAAAGTCAGG
651 CTCATCCACC CACCAGCTGG AGGTCCACAT TGAGAGCAAA
CACGATGAAG
701 CTGAGGATGC CAGCAGTGGA CGGCTAGAAA TAGATACCAG
TGCCCAGAAT
751 AAGCATAACC CTTTGCTCAT CGTGTTTTCT GATGACCAAA
GCAGTGACAA
801 GGAGAGGAAG GAGGAACTGA ATGAAATGAT TTCCCATGAG
CAACTTCCAG
851 AGCTGGACAA CTTGGGCCTG GATAGCTTTT CCAGTGGACC
TGGGGAAGAG
901 GCTTTGTTGC AGATGAGATC AAACATCATC TATGACTCCA
CTGCCCGAAT
951 CAGAACCGGT GGTGGAACTC ACACATGCCC ACCGTGCCCA
GCACCTGAAC
1001 TCCTGGGGGG ACCGTCAGTC TTCCTCTTCC CCCCAAAACC
CAAGGACACC
1051 CTCATGATCT CCCGGACCCC TGAGGTCACA TGCGTGGTGG
TGGACGTGAG
1101 CCACGAAGAC CCTGAGGTCA AGTTCAACTG GTACGTGGAC
GGCGTGGAGG
1151 TGCATAATGC CAAGACAAAG CCGCGGGAGG AGCAGTACAA
CAGCACGTAC
1201 CGTGTGGTCA GCGTCCTCAC CGTCCTGCAC CAGGACTGGC
TGAATGGCAA
1251 GGAGTACAAG TGCAAGGTCT CCAACAAAGC CCTCCCAGCC
CCCATCGAGA
1301 AAACCATCTC CAAAGCCAAA GGGCAGCCCC GAGAACCACA
GGTGTACACC
1351 CTGCCCCCAT CCCGGGAGGA GATGACCAAG AACCAGGTCA
GCCTGACCTG
1401 CCTGGTCAAA GGCTTCTATC CCAGCGACAT CGCCGTGGAG
TGGGAGAGCA
1451 ATGGGCAGCC GGAGAACAAC TACAAGACCA CGCCTCCCGT
GCTGGACTCC
1501 GACGGCTCCT TCTTCCTCTA TAGCAAGCTC ACCGTGGACA
AGAGCAGGTG
1551 GCAGCAGGGG AACGTCTTCT CATGCTCCGT GATGCATGAG
GCTCTGCACA
1601 ACCACTACAC GCAGAAGAGC CTCTCCCTGT CTCCGGGTAA ATGA
(SEQ ID NO: 57)

A processed BMP10pro(22-315)-hFc fusion protein (SEQ ID NO: 58) is as follows and may optionally be provided with lysine removed from the C-terminus.

1 SPIMNL EQSPLEEDMS LFGDVFSEQD
51 GVDFNTLLQS MKDEFLKTLN LSDIPTQDSA KVDPPEYMLE LYNKFATDRT
101 SMPSANIIRS FKNEDLFSQP VSFNGLRKYP LLFNVSIPHH EEVIMAELRL
151 YTLVQRDRMI YDGVDRKITI FEVLESKGDN EGERNMLVLV SGEIYGTNSE
201 WETFDVTDAI RRWQKSGSST HQLEVHIESK HDEAEDASSG RLEIDTSAQN
251 KHNPLLIVFS DDQSSDKERK EELNEMISHE QLPELDNLGL DSFSSGPGEE
301 ALLQMRSNII YDSTARIRTG GGTHTCPPCP APELLGGPSV FLFPPKPKDT
351 LMISRTPEVT CVVVDVSHED PEVKFNWYVD GVEVHNAKTK PREEQYNSTY
401 RVVSVLTVLH QDWLNGKEYK CKVSNKALPA PIEKTISKAK GQPREPQVYT
451 LPPSREEMTK NQVSLTCLVK GFYPSDIAVE WESNGQPENN YKTTPPVLDS
501 DGSFFLYSKL TVDKSRWQQG NVFSCSVMHE ALHNHYTQKS LSLSPGK (SEQ
ID NO: 58)

The BMP propeptide domain is underlined.

Another BMP10 propeptide-Fc fusion protein was generated comprising a greater C-terminal truncation of the human BMP10 propeptide domain (amino acids 22-312 of SEQ ID NO: 1) fused to a human immunoglobulin G1 Fc domain with an optional linker. This fusion protein was designated as BMP10pro(22-312)-hFc. A similar fusion protein was generated using a mouse immunoglobulin G1 Fc domain, which is designated as BMP10pro(22-312)-mFc. Signal sequences for use with the BMP10pro(22-312)-Fc fusion proteins that were considered include, for example, the native human BMP10 precursor leader, honeybee mellitin, and TPA leader.

The human BMP10pro(22-312)-hFc polypeptide sequence (SEQ ID NO: 59) with a TPA leader is shown below:

1 MDAMKRGLCC VLLLCGAVFV SPGASPIMNL EQSPLEEDMS LFGDVFSEQD
51 GVDFNTLLQS MKDEFLKTLN LSDIPTQDSA KVDPPEYMLE LYNKFATDRT
101 SMPSANIIRS FKNEDLFSQP VSFNGLRKYP LLFNVSIPHH EEVIMAELRL
151 YTLVQRDRMI YDGVDRKITI FEVLESKGDN EGERNMLVLV SGEIYGTNSE
201 WETFDVTDAI RRWQKSGSST HQLEVHIESK HDEAEDASSG RLEIDTSAQN
251 KHNPLLIVFS DDQSSDKERK EELNEMISHE QLPELDNLGL DSFSSGPGEE
301 ALLQMRSNII YDSTATGGGT HTCPPCPAPE LLGGPSVFLF PPKPKDTLMI
351 SRTPEVTCVV VDVSHEDPEV KFNWYVDGVE VHNAKTKPRE EQYNSTYRVV
401 SVLTVLHQDW LNGKEYKCKV SNKALPAPIE KTISKAKGQP REPQVYTLPP
451 SREEMTKNQV SLTCLVKGFY PSDIAVEWES NGQPENNYKT TPPVLDSDGS
501 FFLYSKLTVD KSRWQQGNVF SCSVMHEALH NHYTQKSLSL SPGK (SEQ ID
NO: 59)

The BMP propeptide domain is underlined. The amino acid sequence of SEQ ID NO: 59 may optionally be provided with lysine removed from the C-terminus.

This BMP10pro(22-312)-hFc fusion protein is encoded by the following nucleic acid sequence (SEQ ID NO: 60):

1 ATGGATGCAA TGAAGAGAGG GCTCTGCTGT GTGCTGCTGC TGTGTGGAGC
51 AGTCTTCGTT TCGCCCGGCG CCAGCCCCAT CATGAACCTA GAGCAGTCTC
101 CTCTGGAAGA AGATATGTCC CTCTTTGGTG ATGTTTTCTC AGAGCAAGAC
151 GGTGTCGACT TTAACACACT GCTCCAGAGC ATGAAGGATG AGTTTCTTAA
201 GACACTAAAC CTCTCTGACA TCCCCACGCA GGATTCAGCC AAGGTGGACC
251 CACCAGAGTA CATGTTGGAA CTCTACAACA AATTTGCAAC AGATCGGACC
301 TCCATGCCCT CTGCCAACAT CATTAGGAGT TTCAAGAATG AAGATCTGTT
351 TTCCCAGCCG GTCAGTTTTA ATGGGCTCCG AAAATACCCC CTCCTCTTCA
401 ATGTGTCCAT TCCTCACCAT GAAGAGGTCA TCATGGCTGA ACTTAGGCTA
451 TACACACTGG TGCAAAGGGA TCGTATGATA TACGATGGAG TAGACCGGAA
501 AATTACCATT TTTGAAGTGC TGGAGAGCAA AGGGGATAAT GAGGGAGAAA
551 GAAACATGCT GGTCTTGGTG TCTGGGGAGA TATATGGAAC CAACAGTGAG
601 TGGGAGACTT TTGATGTCAC AGATGCCATC AGACGTTGGC AAAAGTCAGG
651 CTCATCCACC CACCAGCTGG AGGTCCACAT TGAGAGCAAA CACGATGAAG
701 CTGAGGATGC CAGCAGTGGA CGGCTAGAAA TAGATACCAG TGCCCAGAAT
751 AAGCATAACC CTTTGCTCAT CGTGTTTTCT GATGACCAAA GCAGTGACAA
801 GGAGAGGAAG GAGGAACTGA ATGAAATGAT TTCCCATGAG CAACTTCCAG
851 AGCTGGACAA CTTGGGCCTG GATAGCTTTT CCAGTGGACC TGGGGAAGAG
901 GCTTTGTTGC AGATGAGATC AAACATCATC TATGACTCCA CTGCCACCGG
951 TGGTGGAACT CACACATGCC CACCGTGCCC AGCACCTGAA CTCCTGGGGG
1001 GACCGTCAGT CTTCCTCTTC CCCCCAAAAC CCAAGGACAC CCTCATGATC
1051 TCCCGGACCC CTGAGGTCAC ATGCGTGGTG GTGGACGTGA GCCACGAAGA
1101 CCCTGAGGTC AAGTTCAACT GGTACGTGGA CGGCGTGGAG GTGCATAATG
1151 CCAAGACAAA GCCGCGGGAG GAGCAGTACA ACAGCACGTA CCGTGTGGTC
1201 AGCGTCCTCA CCGTCCTGCA CCAGGACTGG CTGAATGGCA AGGAGTACAA
1251 GTGCAAGGTC TCCAACAAAG CCCTCCCAGC CCCCATCGAG AAAACCATCT
1301 CCAAAGCCAA AGGGCAGCCC CGAGAACCAC AGGTGTACAC CCTGCCCCCA
1351 TCCCGGGAGG AGATGACCAA GAACCAGGTC AGCCTGACCT GCCTGGTCAA
1401 AGGCTTCTAT CCCAGCGACA TCGCCGTGGA GTGGGAGAGC AATGGGCAGC
1451 CGGAGAACAA CTACAAGACC ACGCCTCCCG TGCTGGACTC CGACGGCTCC
1501 TTCTTCCTCT ATAGCAAGCT CACCGTGGAC AAGAGCAGGT GGCAGCAGGG
1551 GAACGTCTTC TCATGCTCCG TGATGCATGA GGCTCTGCAC AACCACTACA
1601 CGCAGAAGAG CCTCTCCCTG TCTCCGGGTA AATGA (SEQ ID NO: 60)

A processed BMP10pro(22-312)-hFc fusion protein (SEQ ID NO: 61) is as follows and may optionally be provided with lysine removed from the C-terminus.

1 SPIMNL EQSPLEEDMS LFGDVFSEQD
51 GVDFNTLLQS MKDEFLKTLN LSDIPTQDSA KVDPPEYMLE LYNKFATDRT
101 SMPSANIIRS FKNEDLFSQP VSFNGLRKYP LLFNVSIPHH EEVIMAELRL
151 YTLVQRDRMI YDGVDRKITI FEVLESKGDN EGERNMLVLV SGEIYGTNSE
201 WETFDVTDAI RRWQKSGSST HQLEVHIESK HDEAEDASSG RLEIDTSAQN
251 KHNPLLIVFS DDQSSDKERK EELNEMISHE QLPELDNLGL DSFSSGPGEE
301 ALLQMRSNII YDSTATGGGT HTCPPCPAPE LLGGPSVFLF PPKPKDTLMI
351 SRTPEVTCVV VDVSHEDPEV KFNWYVDGVE VHNAKTKPRE EQYNSTYRVV
401 SVLTVLHQDW LNGKEYKCKV SNKALPAPIE KTISKAKGQP REPQVYTLPP
451 SREEMTKNQV SLTCLVKGFY PSDIAVEWES NGQPENNYKT TPPVLDSDGS
501 FFLYSKLTVD KSRWQQGNVF SCSVMHEALH NHYTQKSLSL SPGK (SEQ ID
NO: 61)

The BMP propeptide domain is underlined.

The BMP10pro-Fc fusion proteins described above may be expressed and purified from a COS or CHO cell line to give rise to a homodimeric BMP10pro-Fc fusion protein complex.

Purification of various BMP10pro-Fc fusion proteins complexes can be achieved by a series of column chromatography steps, including, for example, three or more of the following, in any order: protein A chromatography, Q sepharose chromatography, phenylsepharose chromatography, size exclusion chromatography, and cation exchange chromatography. The purification was completed with viral filtration and buffer exchange.

A panel of ligands were screened for potential binding to the C-terminal truncated variants BMP10pro(22-315)-hFc and BMP10pro(22-312)-hFc, as well as the corresponding mouse fusion proteins, using a Biacore™-based binding assay. The BMP10pro-Fc proteins were separately captured onto the system using an anti-Fc antibody, and ligands were injected and allowed to flow over the captured receptor protein. BMP10pro(22-315)-hFc and BMP10pro(22-312)-hFc both showed high affinity for BMP10 and BMP9. Both constructs also displayed high to moderate affinity for BMP6 and BMP5. The mouse Fc equivalent constructs behaved similarly. BMP10pro(22-312)-hFc also displayed high affinity for BMP3b. The affinity of BMP10pro(22-315)-hFc for BMP3b was not assessed.

Using a luciferase reporter construct under the control of four sequential consensus SBE sites (SBE4-luc), which are responsive to Smad 1/4/8-mediated signaling, the mature BMP10-mediated activity in the presence and absence of BMP10pro(22-315)-hFc and BMP10pro(22-312)-hFc, separately, was measured in HMVEC cells. Results are show in the table below

IC₅₀ (BMP 10)

| Fc fusion protein | (ng/ml) | (pM) |
|---|---|---|
| BMP10pro(22-315)-hFc | 61.20 | 516.8 |
| BMP10pro(22-312)-hFc | 17.74 | 149.81 |

The data indicate that BMP10 propeptides can tolerate C-terminal truncations of 1, 2, 3, or 4 amino acids without losing BMP10 antagonizing activity. Moreover, while both fusion proteins were shown to be potent inhibitors of BMP10 activity, the BMP10pro(22-312)-hFc fusion protein was determined to antagonize BMP10 activity threefold or greater than the BMP10pro(22-315)-hFc. The increased activity of BMP10pro(22-312)-hFc is surprising given that it has a greater C-terminal truncation than BMP10pro(22-315)-hFc. Therefore, in certain uses where it is desirable to maximize BMP10 inhibition, a polypeptide comprising a BMP10pro domain ending at residue 312 with respect to SEQ ID NO: 1 may be preferable to a BMP10pro domain ending at any one or residues 313-315 with respect to SEQ ID NO: 1. In addition, to having greater activity, the shorter BMP10pro domain may be preferable in certain therapeutic applications where it is desirable to reduce risk of immune reaction against the BMP10pro polypeptide, i.e., less amino acids reduces the number of potential epitopes that may be recognized by a patient’s immune system.

### Example 5: Effects of BMP10 Propeptide on Pulmonary Hypertension in the Sugen Hypoxia Rat Model

The effects of BMP10pro(22-312)-hFc (“BMP10pro”) and sildenafil (a phosphodiesterase-5 inhibitor approved for the treatment of PAH) were examined using the Sugen Hypoxia (Su/Hx) model of PAH. In accordance, rats received daily doses of semaxanib and were placed in a low oxygen environment (approximately 13% oxygen) to induce PAH 24 hours prior to start of therapy.

Rats were separated into different treatment groups (9 rats per group): 1) control rats (Tris buffered saline administered s.c. as 1 ml/kg, every three days), “Normal”; 2) treatment with semaxanib (200 mg/kg administered s.c. as a single dose daily)/hypoxia and Tris buffered saline (administered s.c. as 1 ml/kg, every three days) (vehicle treatment group), “PBS”; 3) treatment with BMP10pro (10 mg/kg administered s.c. every three days) and semaxanib (200 mg/kg administered s.c. as a single dose daily)/hypoxia, “BMP10pro”; and 4) treatment with sildenafil (30 mg/kg administered orally twice daily) and semaxanib (200 mg/kg administered s.c. as a single dose daily)/hypoxia, “Sildenafil”. Rats were treated for 28 days. Body weights were recorded prior to first dose on Day 1 and then weekly throughout the study.

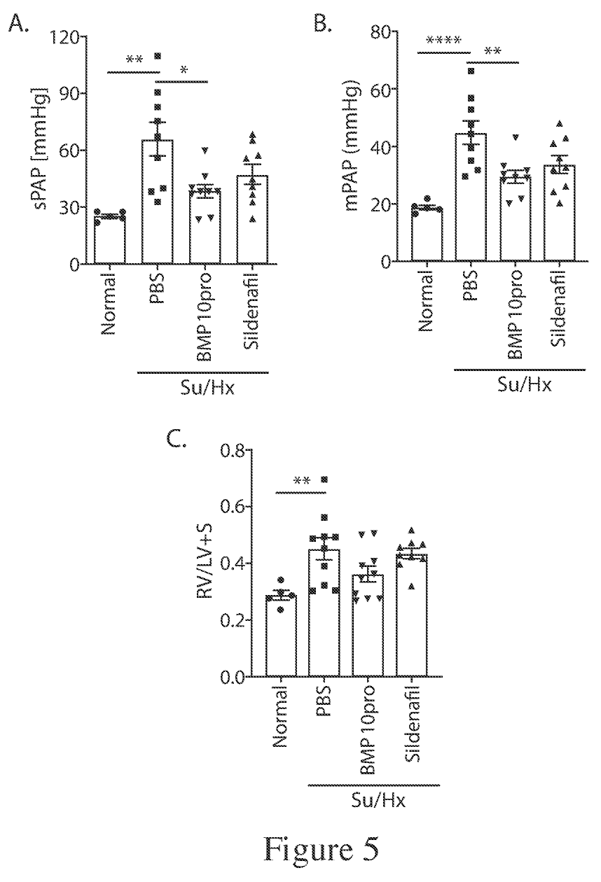

On day 28, rats were anesthetized by an intraperitoneal injection of ketamine/xylazine (80/10 mg/kg). An incision was made in the neck, and a jugular vein was isolated and ligated anteriorly. A fluid-filled pressure catheter was introduced into the right jugular vein to measure pulmonary artery pressure (PAP). FIG. 5A depicts the measured systolic pulmonary artery pressure (sPAP), while FIG. 5B depicts the calculated mean pulmonary artery pressure (mPAP). Another incision was made in the inguinal region, and femoral artery was isolated and ligated anteriorly. A Millar pressure catheter was introduced into a femoral artery to measure systolic arterial pressure, diastolic pressure, and heart rate. Mean arterial pressure and right PAP were monitored using the Notocord HEM (Croissy sur Seine, France) v3.5 data capture system for approximately 5-10 minutes until stable measurements were obtained. During the measurements, rats were maintained at approximately 37° C. on a heating pad and body temperature was monitored throughout the procedure with a rectal temperature probe. At the conclusion of the procedure, rats were euthanized, and the hearts and lungs were removed. The entire heart was weighed. Next, the atria were removed and the left ventricle with septum (LV + S) was separated from the right ventricle (RV). The ventricles were weighed separately. Hypertrophy was assessed, in part, by calculating RV/LV + S (FIG. 5C). The lungs were also weighed.

Compared to “Normal” control animals, semaxanib/hypoxia treated rats (“PBS” animals) were observed to have decreased body weight, elevated PAP, right heart hypertrophy, and increased lung weight, indicating establishment of PAH. Sildenafil treatment (“Sildenafil” animals) reduced mean pulmonary arterial pressure by 24.8%, though this was not statistically significant, and decreased right heart hypertrophy by 3.8% compared to vehicle treated “PBS” animals. Again, BMP10pro treatment (“BMP10pro” animals) was found have greater effects in treating PAH in this model compared to sildenafil. BMP10pro treatment (“BMP10pro” animals) resulted in a reduction of mean pulmonary arterial pressure by 34% and decreased right heart hypertrophy by 19.4% compared to vehicle treated “PBS” animals.

### Example 6: Effects of BMP10 Propeptide on Right Ventricle Dysfunction in the Pulmonary Artery Banding (PAB) Mouse Model

The effects of BMP10pro(22-312)-hFc (“BMP10pro”) were further examined in the pulmonary artery banding (PAB) model of right ventricle (RV) failure. In this model, C57/bl6 male mice were subjected to sham or PAB surgery to induce pressure overload in RV. At 24 hr post-surgery, echocardiography was performed on all mice prior to start of therapy.

Mouse PAB models were prepared in a manner similar to that described in Rockman et al., Proc Natl Acad Sci USA. 1994;91 :2694-8. Briefly, mice were anesthetized with ketamine (100 mg/kg) and xylazine (10 mg/kg). Endotracheal intubation was performed. The endotracheal tube was connected to a small animal ventilator at 100 breaths/min and a tidal volume of 0.2 ml. Animals were placed in the supine position. A midline incision was made and the chest cavity was entered at the second intercostal space to expose the pulmonary artery. A 25-gauge blunt needle was tied against the pulmonary artery. Then the needle was promptly removed. The wound was closed in two layers.

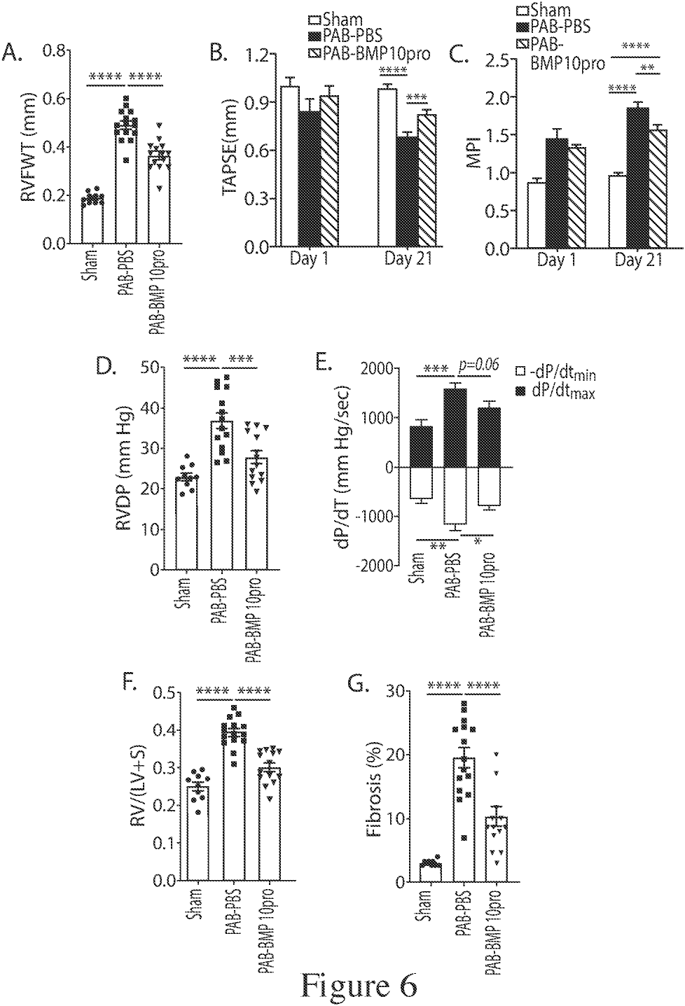

Sham mice were treated with Tris buffered saline administered s.c. as 1 ml/kg, every three days; PAB mice were separated into two treatment groups (15 mice per group): 1) treatment with Tris buffered saline (administered s.c. as 1 ml/kg, every three days) (vehicle treatment group, “PAB-PBS”), 2) treatment with BMP10pro (10 mg/kg administered s.c. every three days), “PAB-BMP10pro”. A third group was separated into a sham surgery group, “Sham”. Mice were treated for 21 days. Body weights were recorded prior to first dose on Day 1 and then weekly throughout the study. Echocardiography was performed on all mice at day 1 and day 21 after PAB surgery to measure RV free wall thickness (RVFWT) (FIG. 6A), decreased tricuspid annular plane systolic excursion (TAPSE) (FIG. 6B), isovolumetric contraction time (ICT), ejection time (ET), and isovolumic relaxation time (IRT). RV global function was estimated by myocardial performance index (MPI) (FIG. 6C), which was calculated by the equation: (ICT+IRT)/ET.

On day 21, mice were anesthetized to measure RV function with ketamine (100 mg/kg) and xylazine (5 mg/kg) at the end of the experiment. The respiration was supported by a small animal ventilator. Thoracotomy was made through 3-4 intercostal space, and the heart was exposed. A Mikro-tip catheter (transducer) was inserted into RV, and RV pressure was recorded. Right ventricular developed pressure (RVDP) (FIG. 6D) and maximal +dp/dt and -dp/dt (FIG. 6E) were calculated by LabChart 7 software.

Compared to sham mice, vehicle treatment group, “PAB-PBS” mice were observed to have right heart hypertrophy (FIG. 6F) and increased RVFWT (FIG. 6A), indicating RV dysfunction. Right heart hypertrophy was assessed by calculating RV/LV + S (FIG. 6F). BMP10pro treatment (“PAB-BMP10Pro”) decreased RV hypertrophy by 23.3%, and reduced RVFWT by 25.3%. In addition, TAPSE was significantly decreased in vehicle-treated “PAB-PBS” mice, suggesting the RV systolic dysfunction, which was improved by BMP10pro treatment (“PAB-BMP10Pro”) (FIG. 6B). MPI was increased on day 1 post PAB surgery and continued to increase on day 21 in vehicle-treated group “PAB-PBS”. BMP10pro treatment (“PAB-BMP10Pro”) significantly reduced MPI on day 21 (FIG. 6C).

RV pressure (RVDP) in PAB mice was significantly increased on day 21 in PAB-PBS animals and BMP10pro treatment (“PAB-BMP10Pro”) decreased RVDP by 24.4% after 3 wks-treatment (FIG. 6D). Similar to RVDP, RV contractility measured by +dp/dt and -dp/dt was significantly increased in PAB-PBS animals, which was significantly inhibited by BMP10pro treatment (“PAB-BMP10Pro”) (FIG. 6E).

In addition, PAB induced cardiac fibrosis and 19.5% of fibrosis area was observed in the vehicle-treated group, which was reduced to 10.3% in the BMP10pro-treated group (“PAB-BMP10Pro”), suggesting the inhibition of RV remodeling by BMP10pro (FIG. 6G).

Together, these data demonstrate that BMP10pro is effective to ameliorate various complications of PAH in the PAB model. In particular, BMP10pro had a greater effect in reducing pulmonary artery pressure and right heart hypertrophy than was observed for sildenafil, which is an approved drug for the treatment of PAH. Furthermore, the data indicate that BMP 10pro inhibited RV remodeling and improved RV function in the pulmonary artery banding model.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

We claim:

1. A method of treating pulmonary hypertension, comprising administering to a patient in need thereof an effective amount of a BMP10 antagonist, wherein the BMP10 antagonist is selected from the group consisting of:
a a polypeptide comprising an amino acid sequence that is at least 90% identical to a sequence that begins at a position corresponding to any one of amino acids 1-6 of SEQ ID NO: 3 and ends at a position corresponding any one of amino acids 292-296 of SEQ ID NO: 3;
b a polypeptide comprising an amino acid sequence that is at least 90% identical to a sequence that begins at a position corresponding to any one of amino acids 1-6 of SEQ ID NO: 3 and ends at a position corresponding any one of amino acids 292-295 of SEQ ID NO: 3, wherein the polypeptide does not comprise the sequence of amino acids RIRR;
c a polypeptide comprising an amino acid sequence that is at least 90% identical to amino acids 2-292 of SEQ ID NO: 3;
d a polypeptide comprising an amino acid sequence that is at least 90% identical to amino acids 2-292 of SEQ ID NO: 3, wherein the polypeptide does not comprise the sequence of amino acids RIRR;
e a polypeptide comprising an amino acid sequence that is at least 90% identical to amino acids 2-295 of SEQ ID NO: 3;
f a polypeptide comprising an amino acid sequence that is at least 90% identical to amino acids 2-295 of SEQ ID NO: 3, wherein the polypeptide does not comprise the sequence of amino acids RIRR;
g a polypeptide comprising an amino acid sequence that is at least 90% identical to a sequence that begins at a position corresponding to any one of amino acids 1-6 of SEQ ID NO: 3 and ends at a position corresponding any one of amino acids 292-295 of SEQ ID NO: 3, wherein the C-terminus of the polypeptide is not R296 of SEQ ID NO: 3;
h a polypeptide comprising an amino acid sequence that is at least 90% identical to a sequence that begins at a position corresponding to any one of amino acids 1-6 of SEQ ID NO: 3 and ends at a position corresponding any one of amino acids 292-296 of SEQ ID NO: 3, wherein the C-terminus of the polypeptide is not R296 of SEQ ID NO: 3;
i a polypeptide comprising an amino acid sequence that is at least 90% identical to amino acids 2-292 of SEQ ID NO: 3, wherein the C-terminus of the polypeptide is not R296 of SEQ ID NO: 3; and
j a polypeptide comprising an amino acid sequence that is at least 90% identical to amino acids 2-295 of SEQ ID NO: 3, wherein the C-terminus of the polypeptide is not R296 of SEQ ID NO: 3.

2. A method of treating or reducing the progression rate and/or severity of one or more complications of pulmonary hypertension, comprising administering to a patient in need thereof an effective amount of a BMP10 antagonist of claim 1.

3. The method of claim 2, wherein the one or more complications of pulmonary hypertension is selected from the group consisting of: smooth muscle and/or endothelial cell proliferation in the pulmonary artery, angiogenesis in the pulmonary artery, dyspnea, chest pain, pulmonary vascular remodeling, right ventricular hypertrophy, and pulmonary fibrosis.

4. The method of claim 2, wherein the pulmonary hypertension is pulmonary arterial hypertension.

5-23. (canceled)

24. The method of claim 1, wherein the BMP10 propeptide polypeptide is a fusion protein comprising an immunoglobulin Fc domain.

25. The method of claim 24, wherein the immunoglobulin Fc domain is an IgG1 Fc immunoglobulin domain.

26. The method of claim 24, wherein the fusion protein comprises a linker domain positioned between theBMP10 propeptide polypeptide domain and the Fc immunoglobulin domain.

27-31. (canceled)

32. The method of claim 1, wherein the method reduces pulmonary vascular resistance in the patient.

33. The method of claim 1, wherein the method increases pulmonary capillary wedge pressure.

34. The method of claim 1, wherein the method increases left ventricular end-diastolic pressure.

35. The method of claim 1, wherein the method increases exercise capacity of the patient.

36-46. (canceled)

47. The method of claim 1, wherein the BMP10 antagonist is selected from the group consisting of:
a a polypeptide comprising an amino acid sequence that is at least 95% identical to amino acids 2-292 of SEQ ID NO: 3;
b a polypeptide comprising an amino acid sequence that is at least 95% identical to amino acids 2-292 of SEQ ID NO: 3, wherein the polypeptide does not comprise the sequence of amino acids RIRR; and
c a polypeptide comprising an amino acid sequence that is at least 95% identical to amino acids 2-292 of SEQ ID NO: 3, wherein the C-terminus of the polypeptide is not R296 of SEQ ID NO: 3.

48. The method of claim 1, wherein the BMP10 antagonist is selected from the group consisting of:
a a polypeptide comprising an amino acid sequence that is at least 97% identical to amino acids 2-292 of SEQ ID NO: 3;
b a polypeptide comprising an amino acid sequence that is at least 97% identical to amino acids 2-292 of SEQ ID NO: 3, wherein the polypeptide does not comprise the sequence of amino acids RIRR; and
c a polypeptide comprising an amino acid sequence that is at least 97% identical to amino acids 2-292 of SEQ ID NO: 3, wherein the C-terminus of the polypeptide is not R296 of SEQ ID NO: 3.

49. The method of claim 1, wherein the BMP10 antagonist is selected from the group consisting of:
a a polypeptide comprising an amino acid sequence that is at least 99% identical to amino acids 2-292 of SEQ ID NO: 3;
b a polypeptide comprising an amino acid sequence that is at least 99% identical to amino acids 2-292 of SEQ ID NO: 3, wherein the polypeptide does not comprise the sequence of amino acids RIRR; and
c a polypeptide comprising an amino acid sequence that is at least 99% identical to amino acids 2-292 of SEQ ID NO: 3, wherein the C-terminus of the polypeptide is not R296 of SEQ ID NO: 3.
